(19) United States
(12) Patent Application Publication
(10) Pub. No.: US 2024/0299558 A1
(43) Pub. Date: Sep. 12, 2024

(54) PROTEOLYSIS TARGETING CHIMERAS AND POLYPHARMACOLOGICAL AGENTS TARGETING BCL-2, AND METHODS OF USE THEREOF

(71) Applicant: University of Maryland, Baltimore, Baltimore, MD (US)

(72) Inventor: Steven FLETCHER, Baltimore, MD (US)

(21) Appl. No.: 18/570,466

(22) PCT Filed: Jun. 17, 2022
(86) PCT No.: PCT/US2022/034071

§ 371 Date: Dec. 14, 2023

Related U.S. Application Data
(60) Provisional application No. 63/212,498, filed on Jun. 18, 2021.

(51) Int. Cl. A61K 47/55; A61K 47/54; A61K 47/60; A61P 35/02

(52) U.S. Cl. CPC A61K 47/55; A61K 47/545; A61K 47/60; A61P 35/02

(57) ABSTRACT

Proteolysis-targeting chimeras (PROTACs) and chimeric compounds that inhibit B-Cell Lymphoma-2 (Bcl-2) protein, and methods of using the same, are provided for treating disease.

### CROSS-REFERENCE TO RELATED APPLICATIONS

This application claims the benefit of priority to U.S. Provisional Application No. 63/212,498, filed Jun. 18, 2021, the entirety of which is incorporated herein by reference.

### STATEMENT AS TO FEDERALLY SPONSORED RESEARCH

This invention was made with government support under CA256314 awarded by the National Institutes of Health. The government has certain rights in the invention.

### FIELD

The disclosure relates generally to Proteolysis-targeting chimeras (PROTACs) and methods of using the same for treating conditions characterized by the overexpression or unregulated activity of the protein B-Cell Lymphoma-2 (Bcl-2).

### BACKGROUND

The B-Cell Lymphoma-2 (Bcl-2) family of proteins regulates the intrinsic apoptosis pathway that is responsible for programmed cell death. The pathway involves protein-protein interactions (PPIs) between pro-apoptotic members of the Bcl-2 family, such as Bim, Bak and Bad, and anti-apoptotic members, such as Bcl-xL and myeloid cell leukemia-1 (Mcl-1). Through conserved hydrophobic crevices, the anti-apoptotic Bcl-2 proteins capture the BH3 α-helical domains of their pro-apoptotic counterparts, effectively “neutralizing” their cell killing functions. Evasion of apoptosis is a hallmark of cancer, and is also one culprit for the development of resistance to current chemo- and radiotherapies.

Mcl-1 overexpression and/or amplification of the Mcl-1 gene immortalizes cells, and has been observed in many human solid tumors, including pancreatic, prostate, cervical, lung and breast cancers, as well as B-cell lymphomas and hematological cancers, including acute myeloid leukemia (AML). While certain Bcl-_{XL}/Bcl-2 inhibitors perform well in clinical trials, their low affinity for Mcl-1 is a contributing factor to the observed resistance of several tumor cell lines. Moreover, the upregulation of Mcl-1 has been directly linked to the reduced efficacy of several FDA-approved anti-cancer chemotherapies. Accordingly, the pharmacologic inhibition of Mcl-1 is an attractive, complementary, and/or adjuvant strategy towards the execution of cancer cells by re-activating apoptosis.

In a similar vein to the inhibition of Bcl-_{xL}, the development of synthetic agents capable of disrupting the interaction between Mcl-1 and the BH3 α-helical “death” domains of pro-apoptotic Bcl-2 proteins could “neutralize” Mcl-1's cell survival role.

### SUMMARY

In an embodiment, the disclosure includes a compound of formula (I), or comprising a substructure of formula (I), or a pharmaceutically acceptable salt, solvate, hydrate, cocrystal, or prodrug thereof:

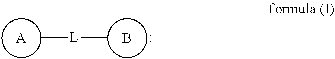

wherein in formula (I):
A comprises a venetoclax moiety;
L is a linking group; and
B comprises a Mcl-1 protein inhibitor moiety, Mcl-1 protein downregulator moiety, or protein degradation moiety, wherein the venetoclax moiety thereof, and the Mcl-1 protein inhibitor moiety, Mcl-1 protein downregulator moiety, or protein degradation moiety thereof are each connected to L at any chemically feasible site.

In an embodiment, the disclosure includes a compound of formula (I), or comprising a substructure of formula (10), or a pharmaceutically acceptable salt, solvate, hydrate, cocrystal, or prodrug thereof:

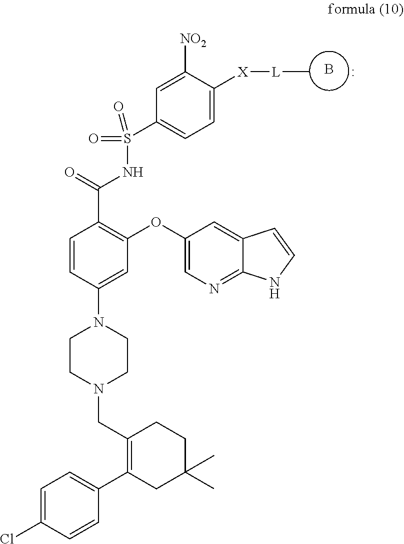

wherein in formula (10):
X is —NH— or

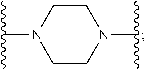 [SMILES: *N1CCN(*)CC1]

and
L is a linking group; and
B comprises a Mcl-1 protein inhibitor moiety, Mcl-1 protein downregulator moiety, or protein degradation moiety, wherein the venetoclax moiety thereof, and the Mcl-1 protein inhibitor moiety, Mcl-1 protein downregulator moiety, or protein degradation moiety thereof are each connected to L at any chemically feasible site.

In an embodiment, the disclosure includes a compound of formula (100) or formula (110), or a pharmaceutically acceptable salt, solvate, hydrate, cocrystal, or prodrug thereof:

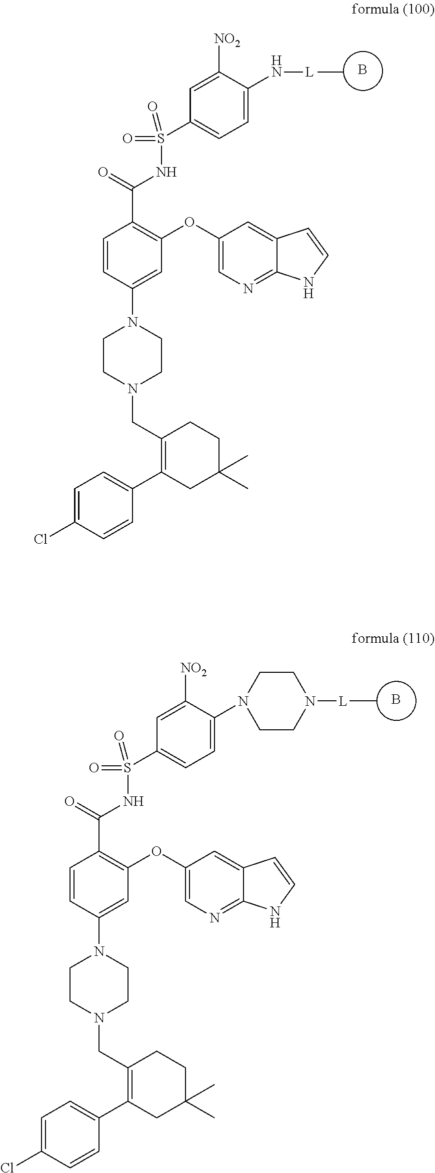

wherein in formula (100) and formula (110):
L is a linking group; and
B comprises a Mcl-1 protein inhibitor moiety, Mcl-1 protein downregulator moiety, or protein degradation moiety, wherein the venetoclax moiety thereof, and the Mcl-1 protein inhibitor moiety, Mcl-1 protein downregulator moiety, or protein degradation moiety thereof are each connected to L at any chemically feasible site. In some embodiments, L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, -cycloalkyl-, -heterocyclyl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR^{b})O—, (O)P(O—)₃, —O(S)P(OR^{b})O—, and (S)P(O—)₃, wherein w is 1 or 2, and R^{b} is independently hydrogen, optionally substituted alkyl, or optionally substituted aryl. In some embodiments, L comprises one or more linking groups selected from —C₁₋₁₀ alkyl-, —O—C₁₋₁₀ alkyl-, —O—, —C(O)—, —N(R^{b})C(O)— wherein R^{b} is hydrogen or optionally substituted alkyl, —C(O)N(R^{b})— wherein R^{b} is hydrogen or optionally substituted alkyl, and —N(R^{b})— wherein R^{b} is hydrogen or optionally substituted alkyl. In some embodiments, L comprises one or more linking groups selected from

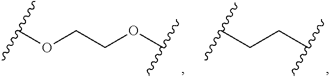

—NHC(O)—, and —C(O)NH—. In some embodiments, L comprises one or more linking groups selected from —NHC(O)—, —C(O)NH—,

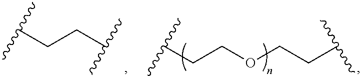

wherein n=1-5. In some embodiments, L comprises one or more linking groups selected from —NHC(O)—, —C(O)NH—,

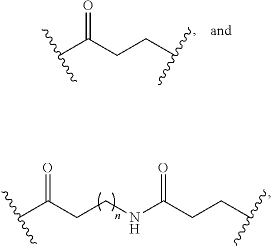

wherein n=0-6.

In some embodiments, B comprises a Mcl-1 protein inhibitor moiety selected from AZD5991, AMG176, and MIK665, and any substructure thereof. In some embodiments, B comprises a Mcl-1 protein downregulator moiety selected from a CDK9 inhibitor, a MEK1/2 inhibitor, a HDAC inhibitor, a FLT3 inhibitor, MDM2 inhibitor, JAK1/2 inhibitor, STAT3 inhibitor, ERK 1/2 inhibitor, dual PI3K/mTOR inhibitor, and any substructure thereof. In some embodiments, the CDK9 inhibitor is selected from alvocidib (flavopiridol), SNS-032, AT7519, TG02, PHA 767491, PHA-793887, PHA-848125, BAY 1143572, BAY 1112054, Cdk9 inhibitor II (CAS 140651-18-9 from Calbiochem), DRB, AZD-5438, dinaciclib, LY2857785, purvalanol B, CDKI-71, CDKI-73, CAN508, FIT-039, CYC065, P276-00, 3,4-dimethyl-5-[2-(4-piperazin-1-yl-phenylamino)-pyrimidin-4-yl]-3H-thiazol-2-one, wogonin, apigenin, chrysin, luteolin, 4-methyl-5-[2-(3-nitroanilino)pyrimidin-4-yl]-1,3-thiazol-2-amine, shRNAs against CDK9, anti-sense mRNA against CDK9, anti-CDK9 antibodies, and substructure thereof. In some embodiments, the MEK1/2 inhibitor is selected from cobimetinib (GDC-0973), PD334581, CI-1040, AZD6244, PD318088, PD98059, RDEA119, 6-Methoxy-7-(3-morpholin-4-yl-propoxy)-4-(4-phenoxy-phenylamino)-quinoline-3-carbonitrile, and 4-[3-Chloro-4-(1-methyl-1H-imidazol-2-ylsulfanyl)-phenylamino]-6-methoxy-7-(3-morpholin-4-yl-propoxy)-quinoline-3-carbonitrile, and any substructure thereof. In some embodiments, the FLT3 inhibitor is selected from gilteritinib, AT7519, quizartinib, midostaurin, crenolanib, FLX-925 also known as AMG-925, G-749, and any substructure thereof. In some embodiments, the MDM2 inhibitor is selected from RG7112 (idasanutlin), PD334581, Nutlin-3a; RG7388; RO5503781; DS-3032b; MI-63; MI-126; MI-122; MI-142; MI-147; MI-18; MI-219; MI-220; MI-221; MI-773; and 3-(4-chlorophenyl)-3-((1-(hydroxymethyl)cyclopropyl)methoxy)-2-(4-nitrobenzyl)isoindolin-1-one; Serdemetan; AM-8553; CGM097, and any substructure thereof. In some embodiments, the HDAC inhibitor is selected from panobinostat, vorinostat, and rocilinostat (ACY-1215), and any substructure thereof. In some embodiments, the JAK 1/2 inhibitor is selected from Ruxolitinib, Baricitinib, Tofacitinib, and any substructure thereof. In some embodiments, the STAT3 inhibitor is selected from WP1066, S3I-201, C1-C10, and any substructure thereof. In some embodiments, the ERK 1/2 inhibitor is selected from WP1066AEZS-131, AEZS-136, BVD-523, SCH-722984, SCH-772984, SCH-900353 (MK-8353), and any substructure thereof. In some embodiments, the dual PI3K/mTOR inhibitor is selected from gedatolisib (PF-05212384; PKI-587), XL765, GDC-0980, BEZ235 (NVP-BEZ235), BGT226, GSK2126458, PF-04691502, and any substructure thereof. In some embodiments, B comprises a moiety selected from AZD5991, SNS-032, AT7519, PD334581, gilteritinib, quizartinib, RG7112 (idasanutlin), panobinostat, AMG176, alvocidib (flavopiridol), cobimetinib (GDC-0973), and any substructure thereof. In some embodiments, B comprises a moiety selected from AZD5991, SNS-032, AT7519, PD334581, gilteritinib, quizartinib, RG7112 (idasanutlin), panobinostat, and any substructure thereof.

In some embodiments, B comprises a moiety selected from

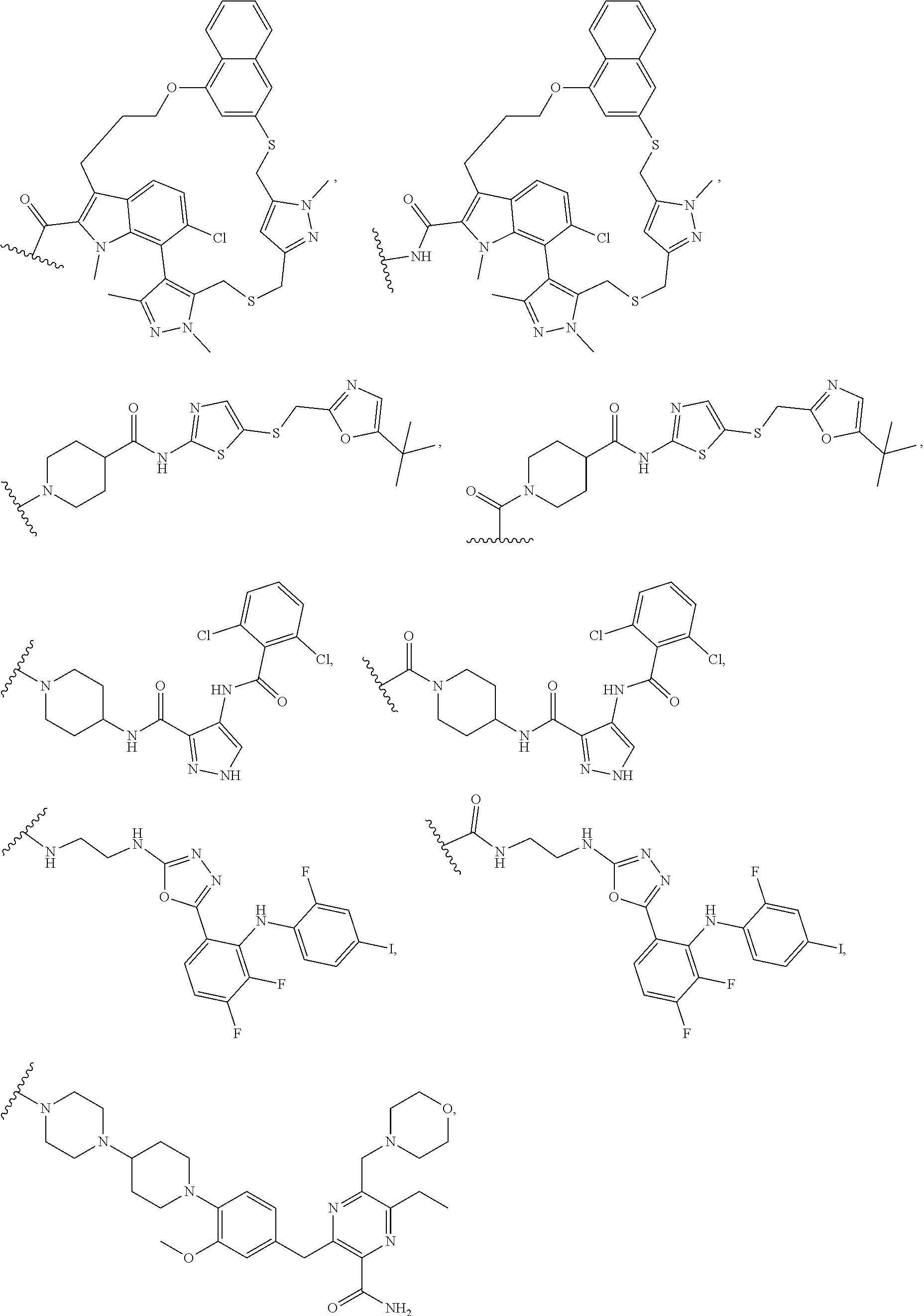

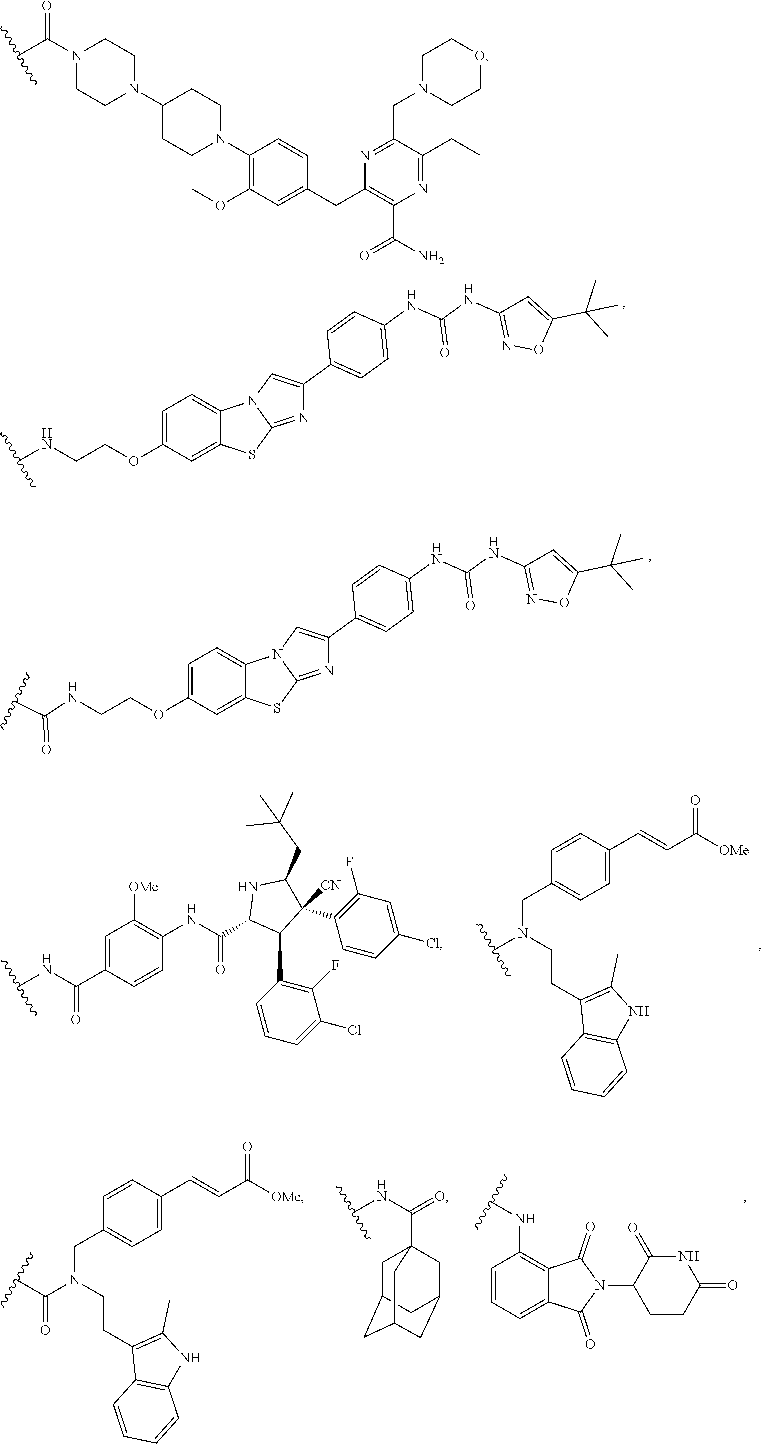

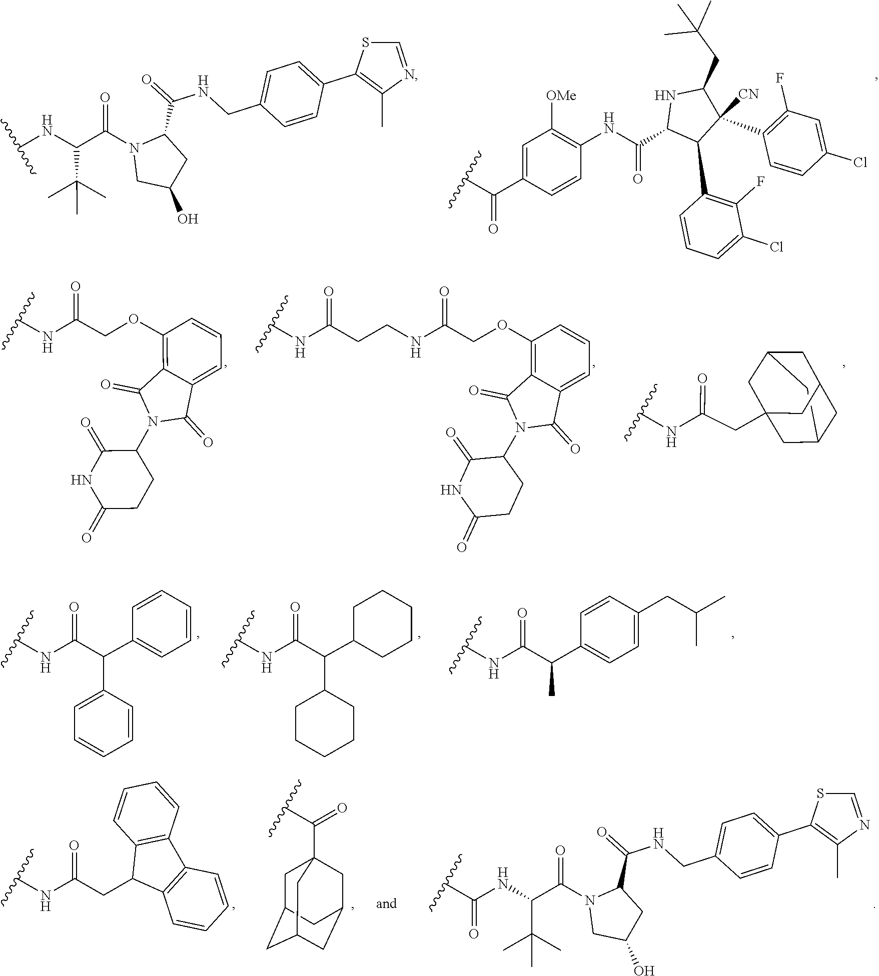

In some embodiments, the protein degradation moiety comprises a hydrophobic tagging group or moiety or a E3 ubiquitin ligase ligand moiety. In some embodiments, the hydrophobic tagging group or moiety comprises an adamantane moiety comprising

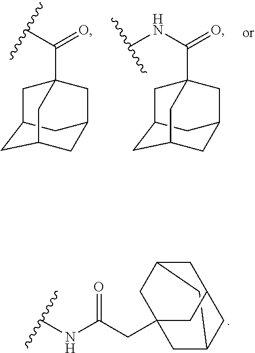 [SMILES: *C(=O)C12CC3CC(CC(C3)C1)C2 ; *NC(=O)C12CC3CC(CC(C3)C1)C2 ; *NC(=O)CC12CC3CC(CC(C3)C1)C2]

In some embodiments, the E3 ubiquitin ligase ligand moiety comprises cereblon (CRBN) ligand, a mouse double minute 2 (MDM2) ligand, a Von Hippel-Lindau (VHL) ligand, or any substructure thereof. In some embodiments, the CRBN ligand is selected from thalidomide, lenalidomide, pomalidomide, and any substructure thereof. In some embodiments, the MDM2 ligand is selected from idasanutlin, RG7112, RG7388, MI 773/SAR 405838, AMG 232, DS-3032b, RO6839921, RO5045337, RO5503781, CGM-097, MK-8242, and any substructure thereof. In some embodiments, the VHL ligand is selected from VHL ligand 1 (VHL-1), VHL ligand 2 (VHL-2), VH032, and any substructure thereof. In some embodiments, the E3 ubiquitin ligase ligand moiety is selected from thalidomide, idasanutlin, VHL ligand 1 (VHL-1), and any substructure thereof. In some embodiments, the E3 ubiquitin ligase ligand moiety comprises

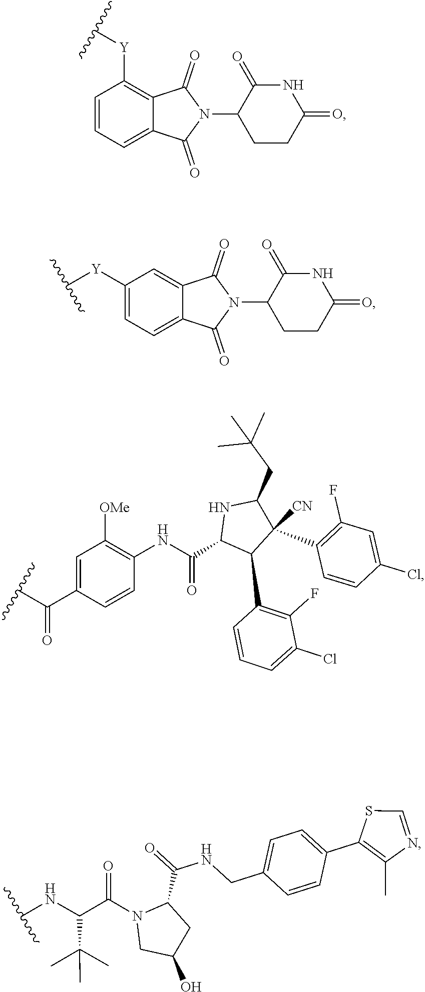

or, wherein Y is selected from —N(R)—, —N(H)—, and —O—, wherein R is optionally substituted alkyl.

In some embodiments, the E3 ubiquitin ligase ligand moiety is selected from

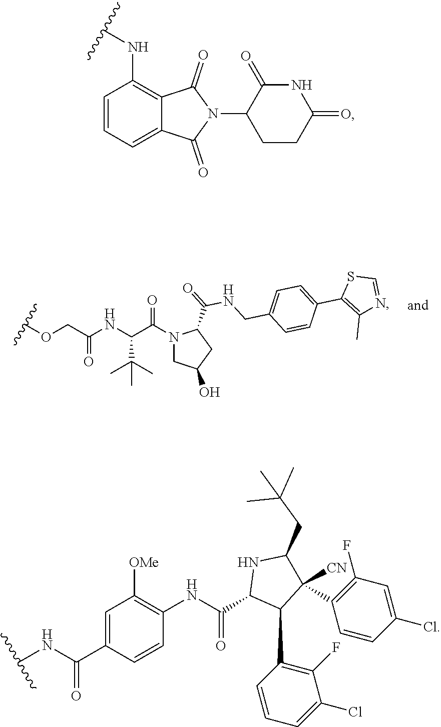 [SMILES: *Nc1cccc2c1C(=O)N(C1CCC(=O)NC1=O)C2=O ; *OCC(=O)N[C@H](C(=O)N1C[C@H](O)C[C@H]1C(=O)NCc1ccc(-c2scnc2C)cc1)C(C)(C)C ; *NC(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1]

In some embodiments, the compound of formula (I), formula (10), formula (100), formula (110), or formula 1001-1170 has a molecular weight not greater than about 2000 g/mol, or about 1900 g/mol, or about 1800 g/mol, or about 1700 g/mol, or about 1600 g/mol, or about 1500 g/mol, or about 1400 g/mol, or about 1300 g/mol, or about 1200 g/mol or about 1100 g/mol, or about 1000 g/mol.

In some embodiments, the compound of formula (100) is a compound of any one of formula 1001-1162, or a pharmaceutically acceptable salt, solvate, hydrate, cocrystal, or prodrug thereof.

In some embodiments, the compound of formula (110) is a compound of any one of formula 1163-1170, or a pharmaceutically acceptable salt, solvate, hydrate, cocrystal, or prodrug thereof.

In one aspect, the disclosure provides a pharmaceutical composition comprising one or more of compounds of any one of formula (I), formula (10), formula (100), formula (110), formula 1001-1170, or a pharmaceutically acceptable salt thereof, and a physiologically compatible carrier medium.

In one aspect, the disclosure provides a pharmaceutical composition for treating or preventing a disease or disorder alleviated by inhibiting Bcl-2 protein activity, the pharmaceutical composition comprising one or more compounds according to any one of formula (I), formula (10), formula (100), formula (110), formula 1001-1170, or a pharmaceutically acceptable salt thereof, and a physiologically compatible carrier medium. In one aspect, the disclosure provides a pharmaceutical composition for treating or preventing a disease or disorder alleviated by inhibiting and/or downregulating Mcl-1 protein activity, the pharmaceutical composition comprising one or more compounds according to any one of formula (I), formula (10), formula (100), formula (110), formula 1001-1170, or a pharmaceutically acceptable salt thereof, and a physiologically compatible carrier medium. In one aspect, the disclosure provides a pharmaceutical composition for treating or preventing a disease or disorder alleviated by both inhibiting Bcl-2 protein activity and inhibiting and/or downregulating Mcl-1 protein activity, the pharmaceutical composition comprising one or more compounds according to any one of formula (I), formula (10), formula (100), formula (110), formula 1001-1170, or a pharmaceutically acceptable salt thereof, and a physiologically compatible carrier medium. In some embodiments, the disease or disorder is cancer. In some embodiments, the cancer is selected from acute myeloid leukemia (AML), chronic lymphocytic leukemia (CLL), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is a blood cancer. In some embodiments, the blood cancer is selected from acute myeloid leukemia (AML), chronic lymphocytic leukemia (CLL), chronic myeloid leukemia (CML), acute lymphocytic lymphoma (ALL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma. In some embodiments, the cancer is acute myeloid leukemia (AML). In some embodiments, the cancer is chronic lymphocytic leukemia (CLL).

In one aspect, the disclosure provides a pharmaceutical composition for treating or preventing from acute myeloid leukemia (AML), the pharmaceutical composition comprising one or more compounds according any one of formula (I), formula (10), formula (100), formula (110), formula 1001-1170, or a pharmaceutically acceptable salt thereof, and a physiologically compatible carrier medium.

In one aspect, the disclosure provides a pharmaceutical composition for treating or preventing from chronic lymphocytic leukemia (CLL), the pharmaceutical composition comprising one or more compounds according any one of formula (I), formula (10), formula (100), formula (110), formula 1001-1170, or a pharmaceutically acceptable salt thereof, and a physiologically compatible carrier medium.

In one aspect, the disclosure provides a method of treating or preventing a disease or disorder alleviated by inhibiting Bcl-2 protein activity in a patient in need of said treatment or prevention, the method comprising administering a therapeutically effective amount of one or more compounds of formula (I), formula (10), formula (100), formula (110), formula 1001-1170, or a pharmaceutically acceptable salt thereof. In some embodiments, the disease or disorder is cancer In one aspect, the disclosure provides a method of treating or preventing a disease or disorder alleviated by inhibiting and/or downregulating Mcl-1 protein activity in a patient in need of said treatment or prevention, the method comprising administering a therapeutically effective amount of one or more compounds of formula (I), formula (10), formula (100), formula (110), formula 1001-1170, or a pharmaceutically acceptable salt thereof. In some embodiments, the disease or disorder is cancer In one aspect, the disclosure provides a method of treating or preventing a disease or disorder alleviated by both inhibiting Bcl-2 protein activity and inhibiting and/or downregulating Mcl-1 protein activity in a patient in need of said treatment or prevention, the method comprising administering a therapeutically effective amount of one or more compounds of formula (I), formula (10), formula (100), formula (110), formula 1001-1170, or a pharmaceutically acceptable salt thereof. In some embodiments, the disease or disorder is cancer. In some embodiments, the cancer is selected from acute myeloid leukemia (AML), chronic lymphocytic leukemia (CLL), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is a blood cancer. In some embodiments, the blood cancer is selected from acute myeloid leukemia (AML), chronic lymphocytic leukemia (CLL), chronic myeloid leukemia (CML), acute lymphocytic lymphoma (ALL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma. In some embodiments, the cancer is acute myeloid leukemia (AML). In some embodiments, the cancer is chronic lymphocytic leukemia (CLL)

In one aspect, the disclosure provides a method of treating or preventing acute myeloid leukemia (AML) in a patient in need of said treatment or prevention, the method comprising administering a therapeutically effective amount of one or more compounds of any one of formula (I), formula (10), formula (100), formula (110), formula 1001-1170, or a pharmaceutically acceptable salt thereof.

In one aspect, the disclosure provides a method of treating or preventing chronic lymphocytic leukemia (CLL) in a patient in need of said treatment or prevention, the method comprising administering a therapeutically effective amount of one or more compounds of any one of formula (I), formula (10), formula (100), formula (110), formula 1001-1170, or a pharmaceutically acceptable salt thereof.

### BRIEF DESCRIPTION OF THE DRAWINGS

The foregoing summary, as well as the following detailed description of embodiments of the disclosure, will be better understood when read in conjunction with the appended drawings and figures.

In the drawings:

FIG. 1A and FIG. 1B illustrate non-limiting general strategies to prepare compounds of the disclosure.

FIG. 2A and FIG. 2B illustrate non-limiting examples of general strategies to prepare compounds of the disclosure. In FIG. 2A, R¹ and L are defined as in FIG. 1A and FIG. 1B, and W=CO₂/Bu or NHBoc. In FIG. 2B, L is defined as in FIG. 1A and FIG. 1B.

FIG. 3A and FIG. 3B illustrate drugs exhibiting synergy with VEN in AML, and in vitro and in vivo in AML.

FIG. 4A and FIG. 4B illustrate non-limiting examples of parent R² drugs with solvent-exposed groups in blue, and the corresponding primed (yellow arrows) R² drugs for conjugation to venetoclax. In some embodiments, drugs with 3° amines are re-synthesized as 1° amines. Protein targets indicated in bold.

FIG. 5A and FIG. 5B illustrate non-limiting examples of chimeric VEN-based inhibitors. In FIG. 5A, L defined as in FIG. 1A and FIG. 1B; MW provided are masses for L=CH₂CH₂. In FIG. 5B, L is defined as in FIG. 1A and FIG. 1B; MW provided are masses for L=CH₂CH₂OCH₂CH₂.

FIG. 6A and FIG. 6B illustrate non-limiting examples of target BCL-2 protein degradation compounds PROTACs. In FIG. 6A, L is defined as in FIG. 1A and FIG. 1B. In FIG. 6B, L defined as in FIG. 1A and FIG. 1B; MW provided are masses for L=CH₂CH₂OCH₂CH₂.

FIG. 7 illustrates a non-limiting example of a reaction scheme for a convergent synthesis to prepare compounds of the disclosure from a common starting material.

FIG. 8 illustrates experimental data demonstrating 48-h alamarBlue cell viability assays of MOLM14 cells in the presence of test compounds compared to venetoclax. The test compounds were prepared through modifications to venetoclax by varying the solvent-exposed morpholine group with this series of “SF” and “CG” PROTACs and hydrophobic-tagged (HyT) denaturants. The absorbance as a fraction of the control is plotted as a function of concentration (nM).

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include the compounds described herein and, more specifically, the one or more compounds of formula (I), formula (10), formula (100), formula (110), or formula 1001-1170. The terms “active pharmaceutical ingredient” and “drug” may also include those compounds described herein that inhibit the Bcl-2 protein and thereby modulate Bcl-2 protein activity and/or downregulate the Mcl-1 protein and thereby modulate Mcl-1 protein activity.

The term “isostere” refers to a group or molecule whose chemical and/or physical properties are similar to those of another group or molecule. A “bioisostere” is a type of isostere and refers to a group or molecule whose biological properties are similar to those of another group or molecule. For example, for the compounds described herein, a carboxylic acid may be replaced by one of the following bioisosteres for carboxylic acids, including, without limitation, alkyl esters (COOR), acylsulfonamides (CONR—SO₂R), hydroxamic acids (CONR—OH), hydroxamates (CONR—OR), tetrazoles, hydroxyisoxazoles, isoxazol-3-ones, and sulfonamides (SO₂NR), where each R may independently represent hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the terms “treat,” “treatment,” and/or “treating” may refer to the management of a disease, disorder, or pathological condition (e.g., pain, a neurological disorder, diarrhea, coughing, muscular tension, and glaucoma), or symptom thereof with the intent to cure, ameliorate, stabilize, prevent, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule. In some embodiments, the biological molecules modulated by the methods and compounds of the disclosure to effect treatment may include one or both of the δ-opioid receptor and the μ-opioid receptor.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” or “physilogically compatible” carrier or carrier medium is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

A “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of formula (I), formula (10), formula (100), formula (110), or formula 1001-1170. The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters (e.g., methyl esters and acetoxy methyl esters). Prodrug esters as employed herein includes esters and carbonates formed by reacting one or more hydroxyls of compounds of the method of the disclosure with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a compound of formula (I), formula (10), formula (100), formula (110), or formula 1001-1170) is a prodrug within the scope of the disclosure. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the disclosure.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where tis 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocycyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where tis 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where tis 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, hydroxamate, acylsulfonamido, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Acylsulfonamide” refers to the group —C(—O)NR^{a}—S(—O)R^{a}, where each R^{a} is independently hydrogen, alkyl, fluoroalkyl, carbocyclyl, carbocyclylalkyl, aryl, aralkyl, heterocycloalkyl, heterocycloalkylalkyl, heteroaryl, or heteroarylalkyl.

“Carboxaldehyde” refers to a —(C═O)H radical.

“Carbonyl” refers to the group —C(═O)—. Carbonyl groups may be substituted with the following exemplary substituents: alkyl, heteroalkyl, alkenyl, alkynyl, cycloalkyl, heterocycloalkyl, hydroxamate, acylsulfonamido, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —NR^{a}—OR^{a}—, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, acylsulfonamido, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, acylsulfonamido, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where tis 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where tis 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where tis 1 or 2), —S(O)ₜOR^{a} (where t is 1 or 2), —S(O)ₜN(R^{a})₂ (where tis 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where tis 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R² of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, amino, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where tis 1 or 2), —S(O)ₜOR^{a} (where t is 1 or 2), —S(O)ₜN(R^{a})₂ (where tis 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where tis 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, acylsulfonamido, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where tis 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, isoxazol-3-one, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a, 7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, a heteroaryl moiety is optionally substituted by one or more substituents which are independently: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where tis 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” or “heterocyclyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“hydroxamate” refers to the —C(O)NR^{a}OR^{a} moiety, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Nitro” refers to the —NO₂ radical.

“Oxa” refers to the —O— radical.

“Oxo” refers to the —O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(+)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both/and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In preferred embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, 3rd Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxamate, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### DETAILED DESCRIPTION

Acute myeloid leukemia (AML), one of the deadliest human cancers, most frequently occurs in older adults, and outcomes for patients over 60 years old are poor, with <2.5% alive 10 years after diagnosis. Lower-intensity chemotherapies are typically administered to frail seniors, including venetoclax (VEN) combined with a hypomethylating agent (HMA), azacytidine or decitabine. However, the median overall survival with this treatment is only 17.5 months, highlighting the urgent need for more effective low-toxicity AML treatments.

The anti-apoptotic members of the B cell lymphoma-2 (BCL-2) family, most commonly BCL-2 itself and/or myeloid cell leukemia 1 (MCL-1) and/or BCL-xL, promote the survival of AML cells (as well as other cancer cell type) by disarming cell-killing via pro-apoptotic sister proteins, seizing them via their BCL-2 homology domain-3 (BH3) death domains. Decades of research culminated in the discovery of VEN, a BH3 mimetic that specifically disrupts binding of BCL-2 to pro-apoptotic proteins. With BCL-2 incapacitated, the multidomain pro-apoptotic proteins BCL-2 antagonist/killer 1 (BAK) and BCL-2 associated X (BAX) trigger mitochondrial outer membrane permeabilization and cell death through the intrinsic apoptosis pathway.

Development of targeted therapies to treat AML is an area of intense research, and while discovery of VEN was a major achievement, VEN monotherapy had a response rate of only 19% with a 2.5 month median time to progression in a Phase 2 AML clinical trial. Combination therapies have demonstrated improved results. Indeed, the FDA recently approved VEN in combination with HMA or low-dose cytarabine in AML patients aged 75 years and older. However, resistance soon becomes manifest, and the median overall survival is only ˜17 months. There is thus an urgent need to understand mechanisms of resistance and construct strategies to evade them, which may take the form of new drugs or novel combination therapies.

MCL-1 over-expression is the best characterized mechanism of VEN resistance. Suppression of MCL-1 activity, either directly or indirectly by pharmacologic or genetic approaches, can rescue VEN-resistant AML cells. However, several cell types depend on MCL-1 function for survival, including hematopoietic stem cells, cardiomyoctes and hepatocytes; MCL-1 knockout in mice is embryonic lethal. Indeed, Amgen's clinical trial of MCL-1 inhibitor AMG176 was halted “to evaluate a safety signal” for cardiotoxicity, although the clinical trial of AstraZeneca's MCL-1 inhibitor AZD5991 remains ongoing. Combination therapies of VEN plus potent MCL-1 inhibitors (direct or indirect) may thus be associated with toxicity to normal tissues, possibly compounded by concomitant BCL-2 inhibition. Another mechanism of VEN resistance is mutation of the BCL-2 protein, such as F104L-BCL-2, and G101V-BCL-2; the latter binds VEN 180-times less tightly. Development of new BCL-2 inhibitors de novo would be an expensive and laborious process; a more fruitful avenue may be through tackling specifically these two major mechanisms of resistance to VEN to restore its efficacy in AML.

Target-specific drugs repeatedly fail in clinical trials, and a recent study suggests the mechanism of action of many oncology drugs is actually through, to some degree, off-target effects; the multi-factorial nature and heterogeneity of cancers necessitates use of drug cocktails. In the last few years, there have been several reports of combination regimens where VEN is administered with a second or third agent to target additional proteins involved in AML, beyond the HMAs mentioned above. In many of these cases, synergism was observed with the drug combination, and VEN-resistant cell lines were sensitive to the combination. However, there are currently no VEN-based polypharmacological agents in clinical trials and no reports of such agents designed to address VEN resistance.

Since small-molecule inhibitors typically bind their target through non-covalent interactions, effective inhibition is beholden to kₒₙ and k_{off} rates. In the relatively new protein degradation technology, an inhibitor of the POI may be fused to a hydrophobic group (tag) or to an E3 ubiquitin ligase recognition ligand. It is believed that the hydrophobic tag induces localized unfolding of the POI, which initiates recruitment of the chaperones HSP40 and HSP70, followed by poly-ubiquitination and degradation by the proteasome. The latter strategy sees the targeted recruitment of the POI to a specific E3 ligase, with subsequent poly-ubiquitination then degradation by the proteasome. Over 100 human E3 ligases have been identified, although only three have been utilized in PROTACs: cereblon (CRBN), murine double minute 2 (MDM2) and von Hippel-Lindau (VHL). Each of these can be selectively targeted with thalidomide, idasanutlin and VHL ligand 1, respectively. Phase 1 trial of Arvinas's lead PROTAC ARV-110 (structure undisclosed but patents suggest MW>1000) has recently begun in prostate cancer.

In one aspect of the disclosure, VEN is synthetically modified VEN in order to directly target two of the most significant mechanisms of VEN resistance: (1) MCL-1 upregulation, and (2) BCL-2 mutation.

In one aspect, the present disclosure provides novel PROTACs and hybrid compounds that function as inhibitors of Bcl-2 and/or modulators Mcl-1 and are useful in treating diseases or disorders associated with dysregulation of Bcl-2 and/or Mcl-1. In some embodiments, the PROTACs and/or hybrid compounds of the disclosure bind to proteins involving the regulation of the oncoprotein, and recruit them to the proteasome for destruction. In some embodiments, the PROTACs and/or hybrid compounds of the disclosure are useful as treatments for a range of cancers, and inhibit Bcl-2 and/or downregulate Mcl-1, which can promote tumorigenesis and is responsible for chemoresistance.

Bcl-2 Inhibitors and/or Mcl-1 Downregulators

In one aspect, the disclosure provides compounds that are inhibitors of Bcl-2 protein activity and/or inhibitors and/or downregulators of MCL-1 protein activity. In some embodiments, downregulation of MCL-1 protein activity comprises suppression of Mcl-1 protein activity, partial inhibition of MCL-1 protein activity, or complete inhibition of MCL-1 protein activity. In some embodiments, the compound directly downregulates MCL-1, indirectly downregulates MCL-1, inhibits MCL-1, or upregulates pro-apoptotic BCL-2 proteins

In some embodiments, the compounds described herein bind other targets associated with a disease or disorder, such as AML and/or CLL, resulting in and as a consequence lead to inhibition of Bcl-2 and/or inhibition of MCL-1 and/or downregulation of MCL-1. Any target that results in the inhibition of Bcl-2 and/or inhibition of MCL-1 and/or downregulation of MCL-1 is contemplated by the disclosure. In some embodiments, the compounds bind to one or more of CDK9, MEK 1/2, HDAC, FLT3, MDM2, JAK1/2, STAT3, ERK1/2, and PI3K/mTOR. In some embodiments, the compounds inhibit one or more of CDK9, MEK1/2, HDAC, FLT3, MDM2, JAK1/2, STAT3, ERK 1/2, and PI3K/mTOR.

In some embodiments, the compounds described above may be delivered as listed or as a pharmaceutically acceptable salt, solvate, hydrate, cocrystal, tautomer, or prodrug thereof.

In one aspect, the disclosure provides a compound of formula (I), or comprising a substructure of formula (I), or a pharmaceutically acceptable salt, solvate, hydrate, cocrystal, or prodrug thereof:

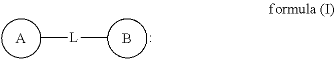

wherein in formula (I):
A comprises a venetoclax moiety;
L is a linking group; and
B comprises a Mcl-1 protein inhibitor moiety, Mcl-1 protein downregulator moiety, or protein degradation moiety, wherein the venetoclax moiety thereof, and the Mcl-1 protein inhibitor moiety, Mcl-1 protein downregulator moiety, or protein degradation moiety thereof are each connected to L at any chemically feasible site.

In one aspect, the disclosure provides a compound of formula (I), or comprising a substructure of formula (10), or a pharmaceutically acceptable salt, solvate, hydrate, cocrystal, or prodrug thereof:

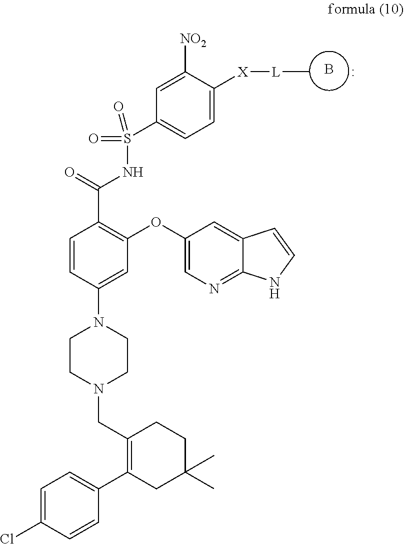

wherein in formula (10):
X is —NH— or

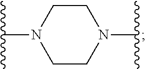 [SMILES: *N1CCN(*)CC1]

and
L is a linking group; and
B comprises a Mcl-1 protein inhibitor moiety, Mcl-1 protein downregulator moiety, or protein degradation moiety, wherein the venetoclax moiety thereof, and the Mcl-1 protein inhibitor moiety, Mcl-1 protein downregulator moiety, or protein degradation moiety thereof are each connected to L at any chemically feasible site.

In an aspect, the disclosure includes a compound of formula (I) or formula (10), or comprising a substructure of formula (100) or formula (110), or a pharmaceutically acceptable salt, solvate, hydrate, cocrystal, or prodrug thereof:

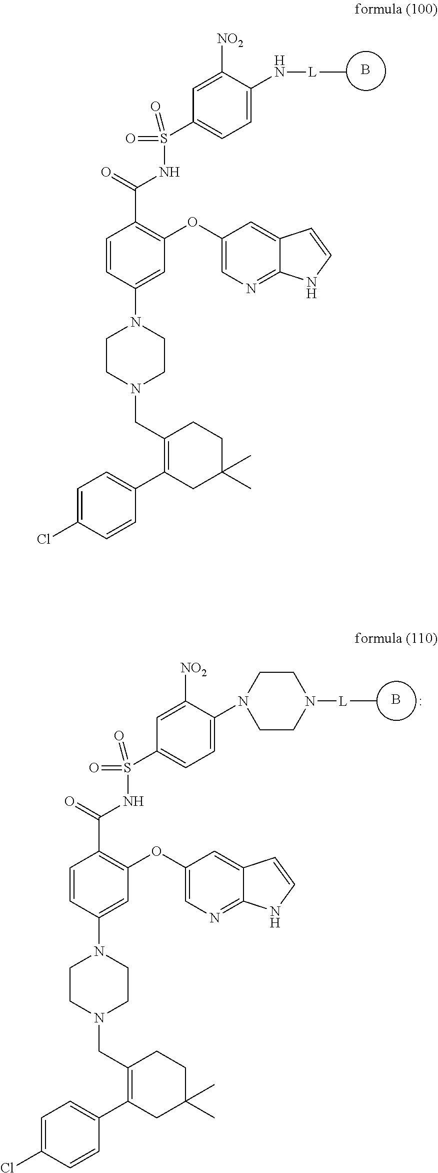

wherein in formula (100) and formula (110):
L is a linking group; and
B comprises a Mcl-1 protein inhibitor moiety, Mcl-1 protein downregulator moiety, or protein degradation moiety, wherein the venetoclax moiety thereof, and the Mcl-1 protein inhibitor moiety, Mcl-1 protein downregulator moiety, or protein degradation moiety thereof are each connected to L at any chemically feasible site.

In some embodiments, B comprises a Mcl-1 protein inhibitor moiety. In some embodiments, the Mcl-1 inhibitor moiety is selected from AZD5991, AMG176, and MIK665, and any substructure thereof. In some embodiments, the Mcl-1 protein downregulator moiety is selected from a CDK9 inhibitor, a MEK1/2 inhibitor, a HDAC inhibitor, a FLT3 inhibitor, MDM2 inhibitor, JAK 1/2 inhibitor, STAT3 inhibitor, ERK 1/2 inhibitor, dual PI3K/mTOR inhibitor, and any substructure thereof.

In some embodiments, B comprises a Mcl-1 protein downregulator moiety. In some embodiments, a Mcl-1 protein inhibitor moiety and/or Mcl-1 protein downregulator moiety provides suppression of Mcl-1 protein activity, partial inhibition of Mcl-1 protein activity, or complete inhibition of Mcl-1 protein activity. Any chemical moiety or substructure that provides inhibition and/or downregulation of the Mcl-1 protein is contemplated by the present disclosure. In some embodiments, the Mcl-1 protein downregulator moiety is an Mcl-1 inhibitor moiety. In some embodiments, the Mcl-1 inhibitor moiety is selected from AZD5991, AMG176, and MIK665, and any substructure thereof. In some embodiments, the Mcl-1 protein downregulator moiety is selected from a CDK9 inhibitor, a MEK1/2 inhibitor, a HDAC inhibitor, a FLT3 inhibitor, MDM2 inhibitor, JAK1/2 inhibitor, STAT3 inhibitor, ERK1/2 inhibitor, dual PI3K/mTOR inhibitor, and any substructure thereof.

Any CDK9 inhibitor is contemplated by the present disclosure. Non-limiting examples of CDK9 inhibitors include alvocidib (flavopiridol), SNS-032, AT7519, TG02, PHA 767491, PHA-793887, PHA-848125, BAY 1143572, BAY 1112054, Cdk9 inhibitor II (CAS 140651-18-9 from Calbiochem), DRB, AZD-5438, dinaciclib, LY2857785, purvalanol B, CDKI-71, CDKI-73, CAN508, FIT-039, CYC065, P276-00, 3,4-dimethyl-5-[2-(4-piperazin-1-yl-phenylamino)-pyrimidin-4-yl]-3H-thiazol-2-one, wogonin, apigenin, chrysin, luteolin, 4-methyl-5-[2-(3-nitroanilino)pyrimidin-4-yl]-1,3-thiazol-2-amine, shRNAs against CDK9, anti-sense mRNA against CDK9, anti-CDK9 antibodies, and any substructure thereof.

Any MEK1/2 inhibitor is contemplated by the present disclosure. Non-limiting examples of MEK1/2 inhibitors include cobimetinib (GDC-0973), PD334581, CI-1040, AZD6244, PD318088, PD98059, RDEA119, 6-Methoxy-7-(3-morpholin-4-yl-propoxy)-4-(4-phenoxy-phenylamino)-quinoline-3-carbonitrile, and 4-[3-Chloro-4-(1-methyl-1H-imidazol-2-ylsulfanyl)-phenylamino]-6-methoxy-7-(3-morpholin-4-yl-propoxy)-quinoline-3-carbonitrile, and any substructure thereof.

Any FLT3 inhibitor is contemplated by the present disclosure. Non-limiting examples of FLT3 inhibitors include gilteritinib, AT7519, quizartinib, midostaurin, crenolanib, FLX-925 also known as AMG-925, G-749, and any substructure thereof.

Any MDM2 inhibitor is contemplated by the present disclosure. Non-limiting examples of MDM2 inhibitors include RG7112 (idasanutlin), PD334581, Nutlin-3a; RG7388; RO5503781; DS-3032b; MI-63; MI-126; MI-122; MI-142; MI-147; MI-18; MI-219; MI-220; MI-221; MI-773; and 3-(4-chlorophenyl)-3-((1-(hydroxymethyl)cyclopropyl)methoxy)-2-(4-nitrobenzyl)isoindolin-1-one; Serdemetan; AM-8553; CGM097, and any substructure thereof.

Any HDAC inhibitor is contemplated by the present disclosure. Non-limiting examples of HDAC inhibitors include panobinostat, vorinostat, and rocilinostat (ACY-1215), and any substructure thereof.

Any JAK1/2 inhibitor is contemplated by the present disclosure. Non-limiting examples of JAK1/2 inhibitors include Ruxolitinib, Baricitinib, and Tofacitinib, and any substructure thereof.

Any STAT3 inhibitor is contemplated by the present disclosure. Non-limiting examples of STAT3 inhibitors include WP1066, S3I-201 and C1-C10, and any substructure thereof.

Any ERK1/2 inhibitor is contemplated by the present disclosure. Non-limiting examples of ERK1/2 inhibitors include WP1066AEZS-131, AEZS-136, BVD-523, SCH-722984, SCH-772984, and SCH-900353 (MK-8353), and any substructure thereof.

Any dual PI3K/mTOR inhibitor is contemplated by the present disclosure. Non-limiting examples of dual PI3K/mTOR inhibitors include gedatolisib (PF-05212384; PKI-587), XL765, GDC-0980, BEZ235 (NVP-BEZ235), BGT226, GSK2126458, and PF-04691502, and any substructure thereof.

In some embodiments, B comprises a moiety selected from AZD5991, SNS-032, AT7519, PD334581, gilteritinib, quizartinib, RG7112 (idasanutlin), panobinostat, AMG176, alvocidib (flavopiridol), cobimetinib (GDC-0973), and any substructure thereof.

In some embodiments, B comprises a moiety selected from AZD5991, SNS-032, AT7519, PD334581, gilteritinib, quizartinib, RG7112 (idasanutlin), panobinostat, and any substructure thereof.

In some embodiments, B comprises a moiety selected from:

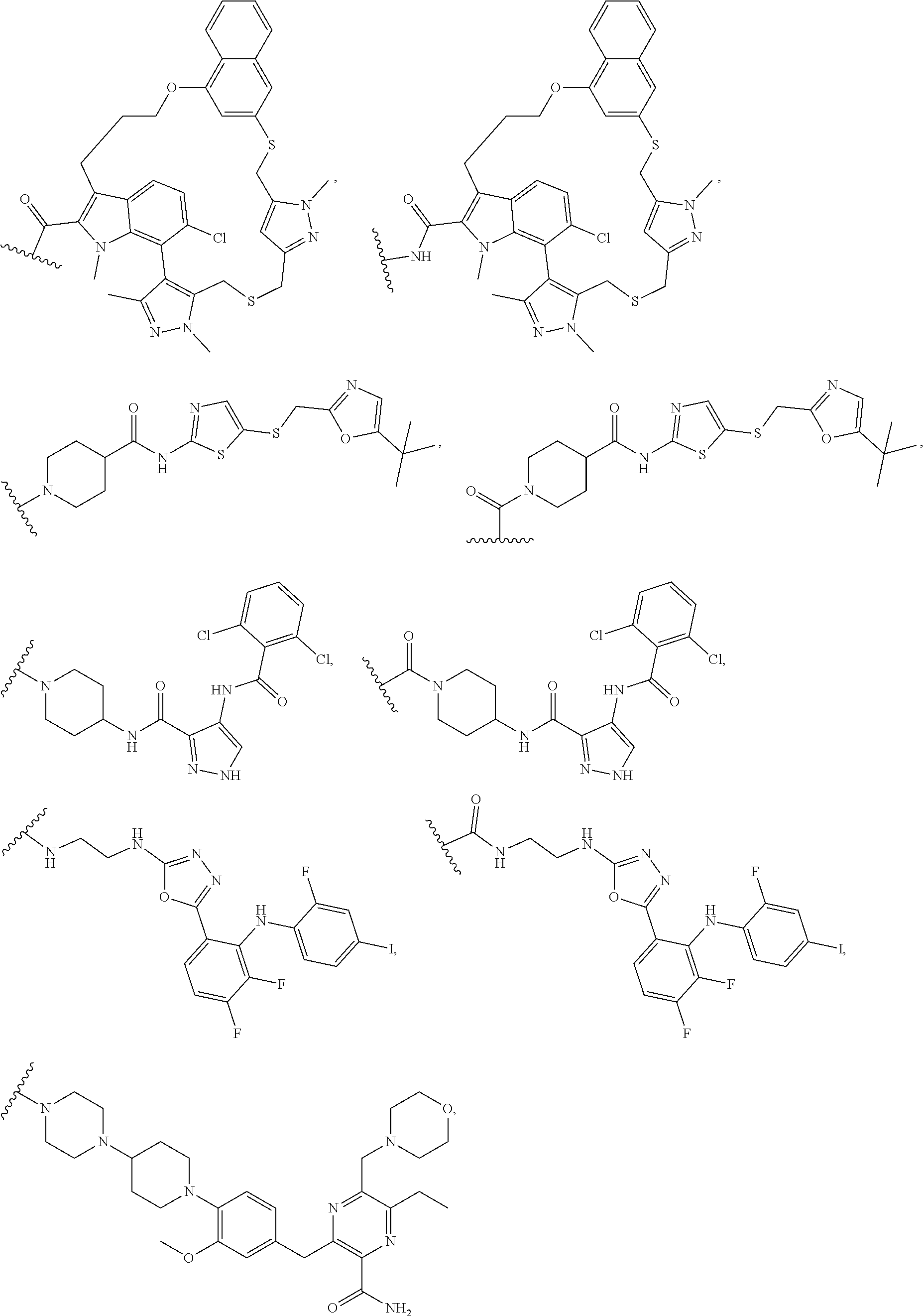

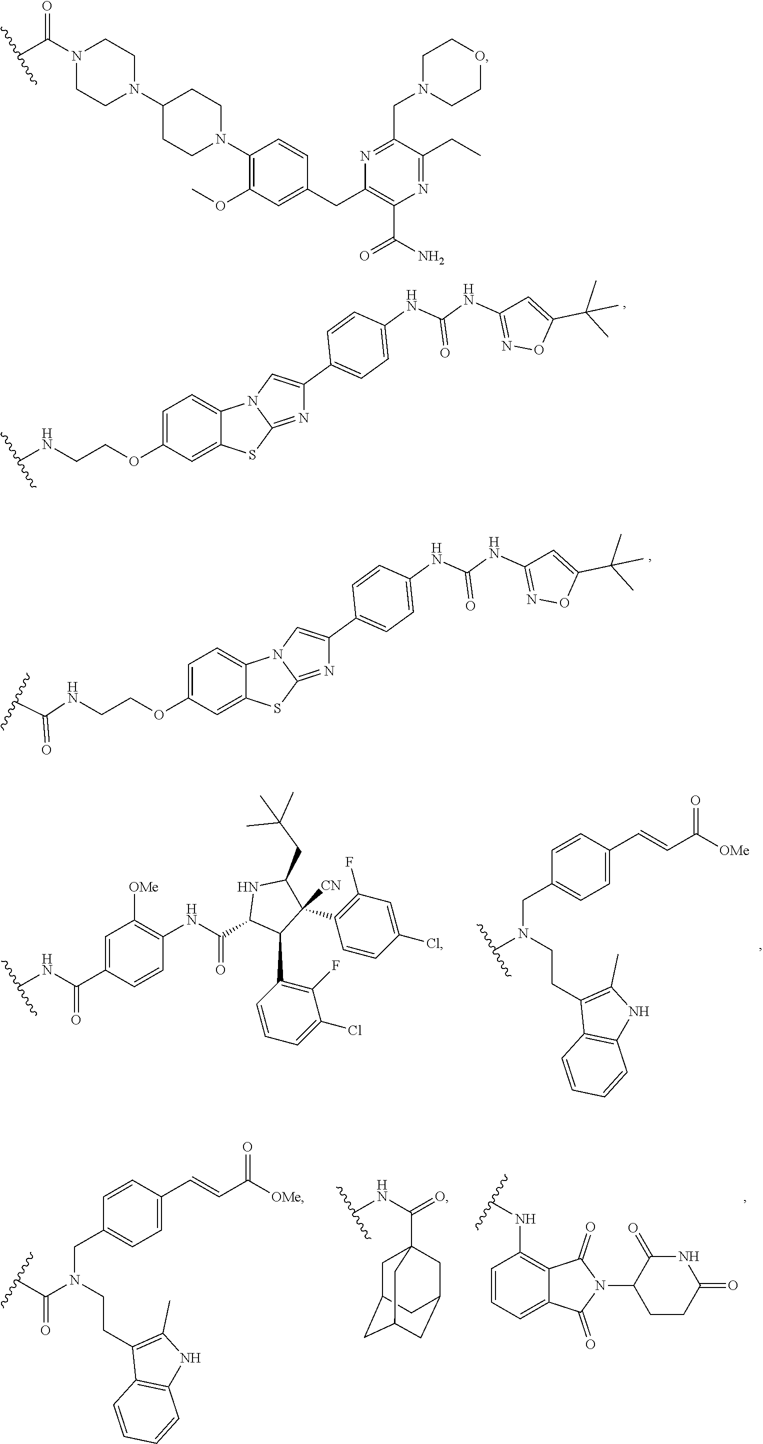

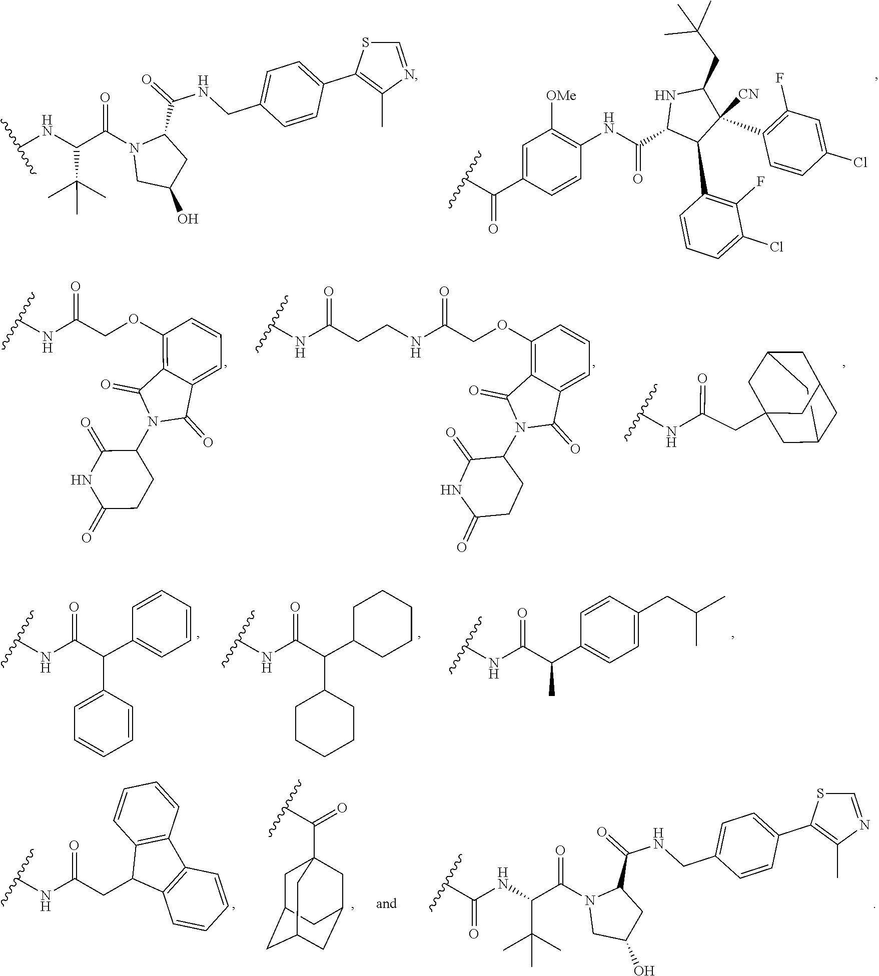

In some embodiments, B is a protein degradation moiety. In some embodiments, wherein the protein degradation moiety comprises an adamantane moiety or an E3 ubiquitin ligase ligand moiety.

In some embodiments, the protein degradation moiety is a hydrophobic tagging group or moiety. Any hydrophobic tag, tagging group, or moiety is contemplated by the disclosure. In some embodiments, the hydrophobic tagging group or moiety comprises an adamantane moiety. In some embodiments, the protein degradation moiety comprises an adamantane moiety. In some embodiments, the adamantane moiety comprises

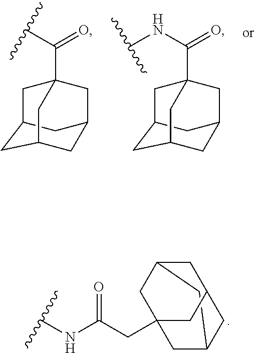 [SMILES: *C(=O)C12CC3CC(CC(C3)C1)C2 ; *NC(=O)C12CC3CC(CC(C3)C1)C2 ; *NC(=O)CC12CC3CC(CC(C3)C1)C2]

In some embodiments, the protein degradation moiety is an E3 ubiquitin ligase ligand moiety. Any E3 ubiquitin ligase ligand moiety is contemplated by the present disclosure. In some embodiments, the E3 ubiquitin ligase ligand moiety comprises cereblon (CRBN) ligand, a mouse double minute 2 (MDM2) ligand, a Von Hippel-Lindau (VHL) ligand, or any substructure thereof.

Any CRBN ligand is contemplated by the present disclosure. Non-limiting examples CRBN ligands include thalidomide, lenalidomide, pomalidomide, and any substructure thereof.

Any MDM2 ligand is contemplated by the present disclosure. Non-limiting examples MDM2 ligands include idasanutlin, RG7112, RG7388, MI 773/SAR 405838, AMG 232, DS-3032b, RO6839921, RO5045337, RO5503781, CGM-097, MK-8242, and any substructure thereof.

Any VHL ligand is contemplated by the present disclosure. Non-limiting examples MDM2 ligands include VHL ligand 1 (VHL-1), VHL ligand 2 (VHL-2), VH032, and any substructure thereof.

In some embodiments, the E3 ubiquitin ligase ligand moiety is selected from thalidomide, idasanutlin, VHL ligand 1 (VHL-1), and any substructure thereof.

In some embodiments, the E3 ubiquitin ligase ligand moiety is selected from:

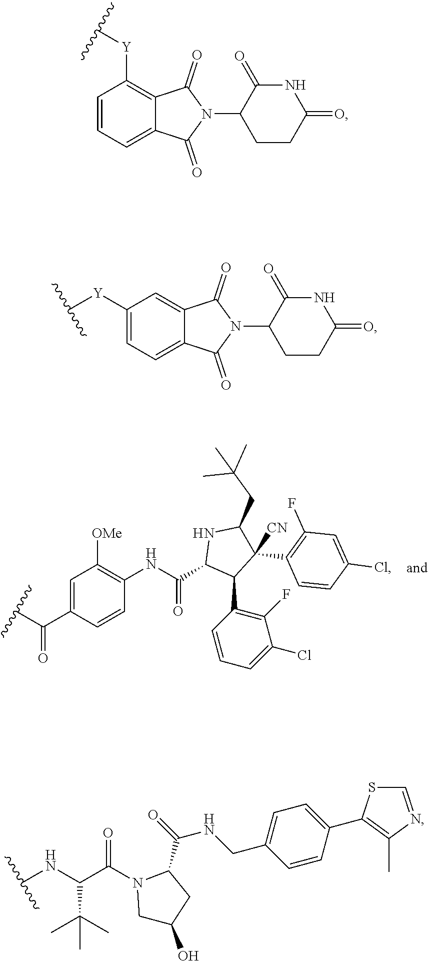

wherein Y is selected from —N(R)—, —N(H)—, and —O—, wherein R is optionally substituted alkyl.

In some embodiments, the E3 ubiquitin ligase ligand moiety is selected from:

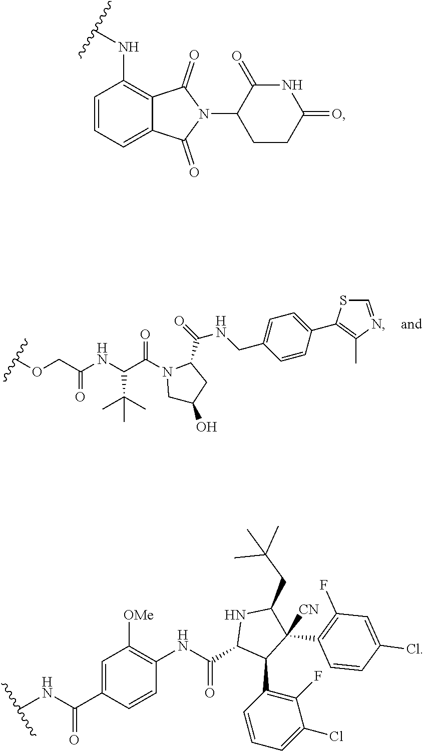

Any organic linker L is contemplated by the present disclosure. In some embodiments, L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, -cycloalkyl-, -heterocyclyl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR^{b})O—, (O)P(O—)₃, —O(S)P(OR^{b})O—, and (S)P(O—)₃, wherein w is 1 or 2, and R^{b} is independently hydrogen, optionally substituted alkyl, or optionally substituted aryl.

In some embodiments, wherein L comprises one or more linking groups selected from —C₁₋₁₀ alkyl-, —O—C₁₋₁₀ alkyl-, —O—, —C(O)—, —N(R^{b})C(O)— wherein R^{b} is hydrogen or optionally substituted alkyl, —C(O)N(R^{b})— wherein R^{b} is hydrogen or optionally substituted alkyl, and —N(R^{b})— wherein R^{b} is hydrogen or optionally substituted alkyl.

In some embodiments, L comprises one or more linking groups selected from

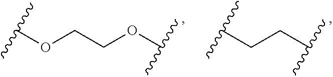

### —NHC(O)—, and —C(O)NH—.

In some embodiments L comprises one or more linking groups selected from —NHC(O)—, —C(O)NH—,

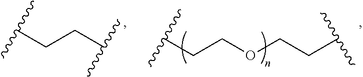

wherein n=1-5.

In some embodiments, L comprises one or more linking groups selected from —NHC(O)—, —C(O)NH—,

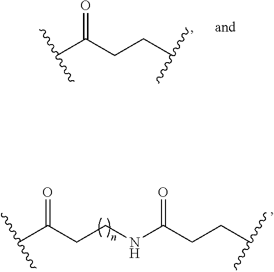

wherein n=0-6.

In some embodiments, the compound of formula (100) is a compound of any one of formula 1001-1162, or a pharmaceutically acceptable salt, solvate, hydrate, cocrystal, or prodrug thereof:

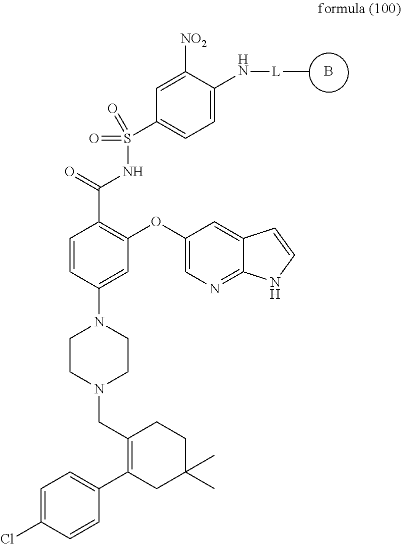

B is

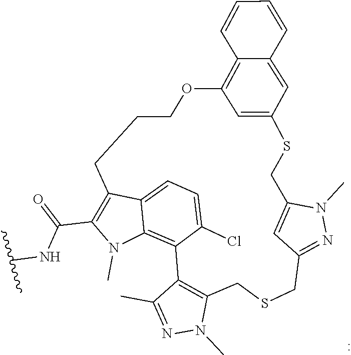 [SMILES: *NC(=O)c1c2c3ccc(Cl)c(c3n1C)-c1c(C)nn(C)c1CSCc1cc(n(C)n1)CSc1cc(c3ccccc3c1)OCCC2]

| Formula No. | L | n |
|---|---|---|
| 1001 | 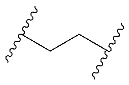 [SMILES: *CC*] | — |
| 1002 | 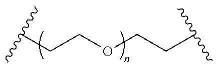 | 1 |
| 1003 | 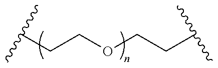 | 2 |
| 1004 | 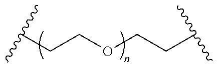 | 3 |
| 1005 | 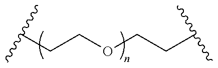 | 4 |
| 1006 | 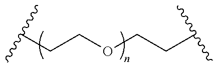 | 5 |

B is

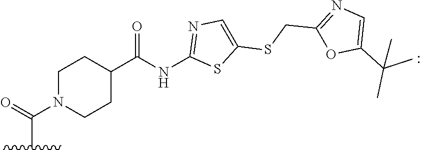 [SMILES: *C(=O)N1CCC(C(=O)Nc2ncc(SCc3ncc(C(C)(C)C)o3)s2)CC1]

| Formula No. | L | n |
|---|---|---|
| 1007 | 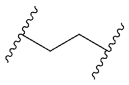 [SMILES: *CC*] | — |
| 1008 | 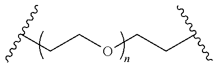 | 1 |
| 1009 | 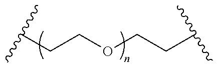 | 2 |
| 1010 | 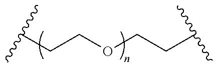 | 3 |
| 1011 | 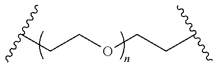 | 4 |
| 1012 | 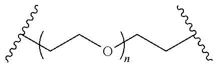 | 5 |

B is

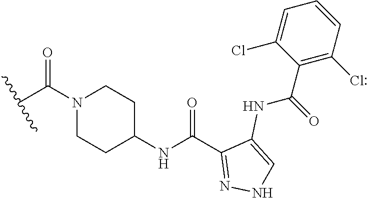

| Formula No. | L | n |
|---|---|---|
| 1013 | 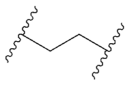 [SMILES: *CC*] | — |
| 1014 | 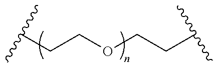 | 1 |
| 1015 | 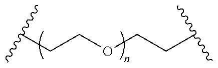 | 2 |
| 1016 | 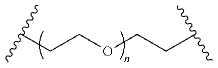 | 3 |
| 1017 | 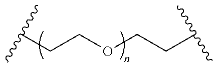 | 4 |
| 1018 | 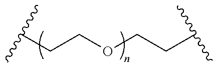 | 5 |

B is

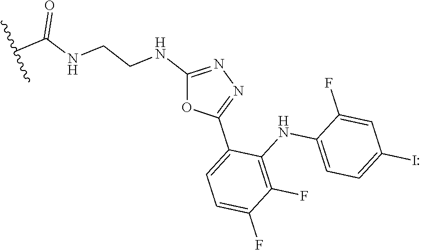

| Formula No. | L | n |
|---|---|---|
| 1019 | 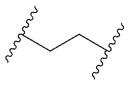 [SMILES: *CC*] | — |
| 1020 | 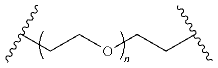 | 1 |
| 1021 | 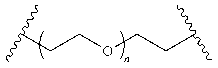 | 2 |
| 1022 | 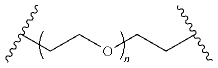 | 3 |
| 1023 | 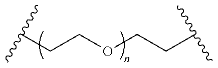 | 4 |
| 1024 | 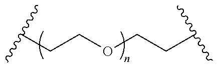 | 5 |

B is

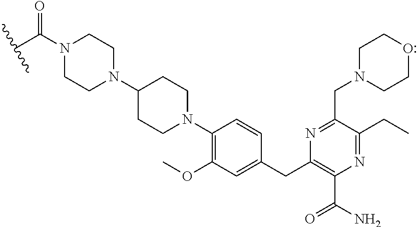

| Formula No. | L | n |
|---|---|---|
| 1025 | 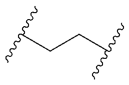 [SMILES: *CC*] | — |
| 1026 | 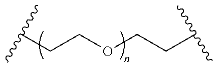 | 1 |
| 1027 | 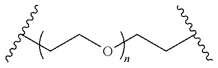 | 2 |
| 1028 | 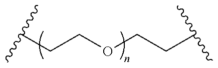 | 3 |
| 1029 | 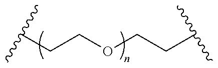 | 4 |
| 1030 | 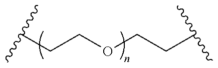 | 5 |

B is

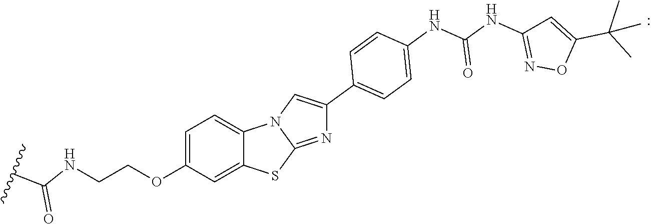 [SMILES: *C(=O)NCCOc1ccc2c(c1)sc1nc(-c3ccc(NC(=O)Nc4cc(C(C)(C)C)on4)cc3)cn12]

| Formula No. | L | n |
|---|---|---|
| 1031 | 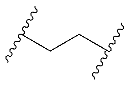 [SMILES: *CC*] | — |
| 1032 | 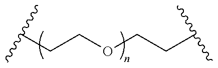 | 1 |
| 1033 | 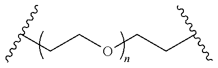 | 2 |
| 1034 | 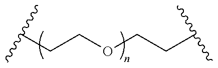 | 3 |
| 1035 | 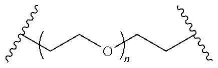 | 4 |
| 1036 | 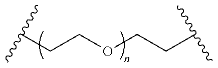 | 5 |

B is

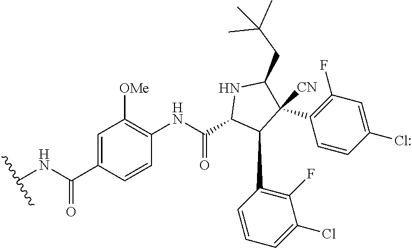

| Formula No. | L | n |
|---|---|---|
| 1037 | 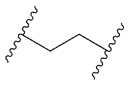 [SMILES: *CC*] | — |
| 1038 | 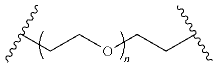 | 1 |
| 1039 | 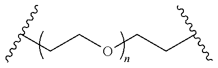 | 2 |
| 1040 | 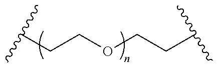 | 3 |
| 1041 | 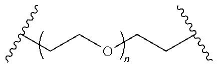 | 4 |
| 1042 | 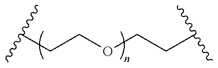 | 5 |

B is

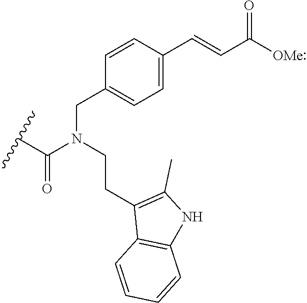

| Formula No. | L | n |
|---|---|---|
| 1043 | 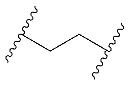 [SMILES: *CC*] | — |
| 1044 | 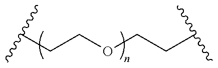 | 1 |
| 1045 | 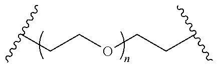 | 2 |
| 1046 | 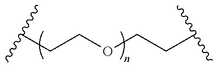 | 3 |
| 1047 | 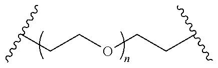 | 4 |
| 1048 | 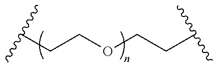 | 5 |

B is

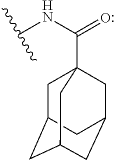

| Formula No. | L | n |
|---|---|---|
| 1049 | 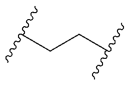 [SMILES: *CC*] | — |
| 1050 | 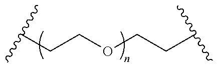 | 1 |
| 1051 | 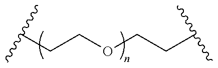 | 2 |
| 1052 | 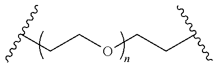 | 3 |
| 1053 | 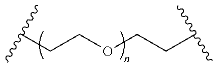 | 4 |
| 1054 | 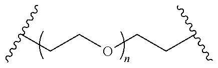 | 5 |

B is

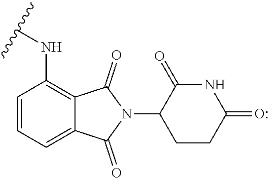

| Formula No. | L | n |
|---|---|---|
| 1055 | 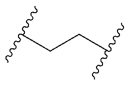 [SMILES: *CC*] | — |
| 1056 | 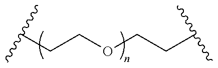 | 1 |
| 1057 | 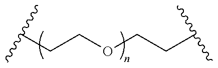 | 2 |
| 1058 | 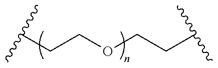 | 3 |
| 1059 | 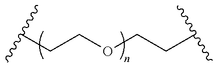 | 4 |
| 1060 | 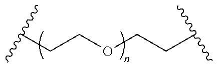 | 5 |

B is

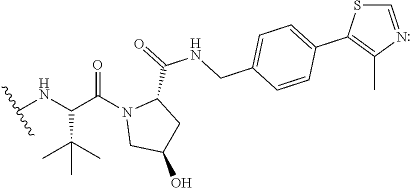

| Formula No. | L | n |
|---|---|---|
| 1061 | 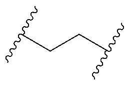 [SMILES: *CC*] | — |
| 1062 | 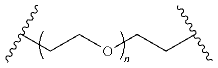 | 1 |
| 1063 | 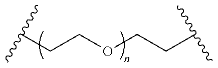 | 2 |
| 1064 | 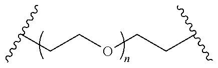 | 3 |
| 1065 | 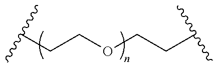 | 4 |
| 1066 | 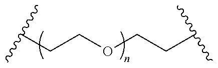 | 5 |

B is

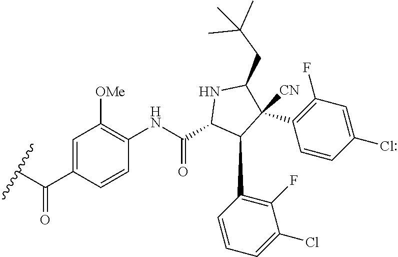

| Formula No. | L | n |
|---|---|---|
| 1067 | 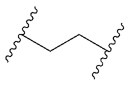 [SMILES: *CC*] | — |
| 1068 | 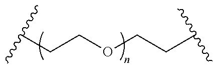 | 1 |
| 1069 | 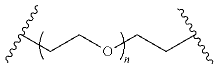 | 2 |
| 1070 | 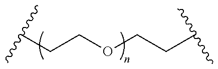 | 3 |
| 1071 | 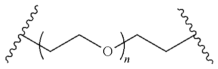 | 4 |
| 1072 | 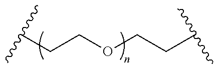 | 5 |

B is

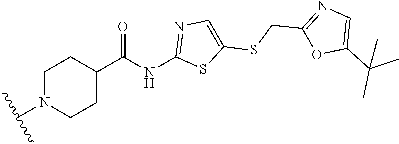 [SMILES: *N1CCC(C(=O)Nc2ncc(SCc3ncc(C(C)(C)C)o3)s2)CC1]

| Formula No. | L | n |
|---|---|---|
| 1073 | 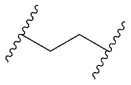 [SMILES: *CC*] | — |
| 1074 | 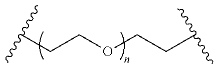 | 1 |
| 1075 | 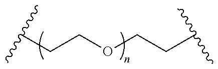 | 2 |
| 1076 | 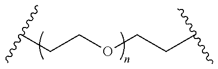 | 3 |
| 1077 | 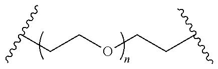 | 4 |
| 1078 | 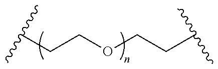 | 5 |

B is

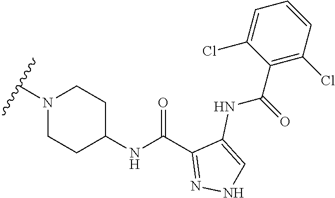 [SMILES: *N1CCC(NC(=O)c2n[nH]cc2NC(=O)c2c(Cl)cccc2Cl)CC1]

| Formula No. | L | n |
|---|---|---|
| 1079 | 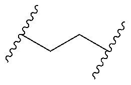 [SMILES: *CC*] | — |
| 1080 | 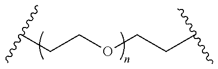 | 1 |
| 1081 | 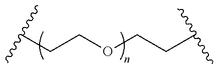 | 2 |
| 1082 | 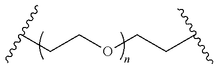 | 3 |
| 1083 | 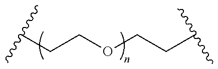 | 4 |
| 1084 | 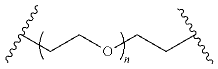 | 5 |

B is

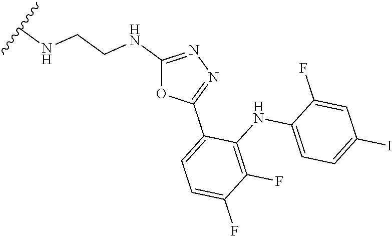 [SMILES: *NCCNc1nnc(-c2ccc(F)c(F)c2Nc2ccc(I)cc2F)o1]

| Formula No. | L | n |
|---|---|---|
| 1085 | 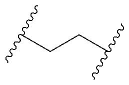 [SMILES: *CC*] | — |
| 1086 | 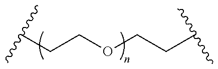 | 1 |
| 1087 | 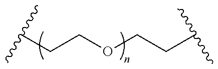 | 2 |
| 1088 | 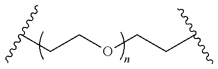 | 3 |
| 1089 | 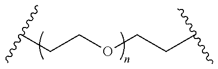 | 4 |
| 1090 | 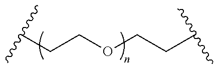 | 5 |

B is

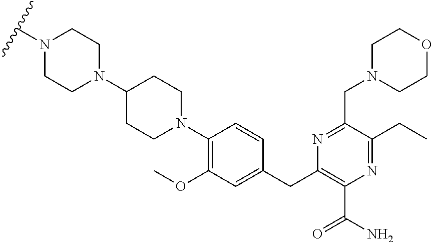 [SMILES: *N1CCN(C2CCN(c3ccc(Cc4nc(CN5CCOCC5)c(CC)nc4C(N)=O)cc3OC)CC2)CC1]

| Formula No. | L | n |
|---|---|---|
| 1091 | 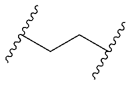 [SMILES: *CC*] | — |
| 1092 | 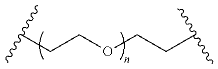 | 1 |
| 1093 | 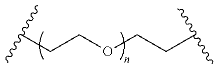 | 2 |
| 1094 | 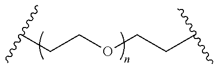 | 3 |
| 1095 | 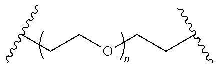 | 4 |
| 1096 | 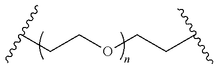 | 5 |

B is

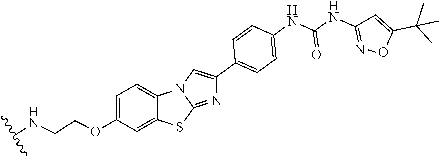 [SMILES: *NCCOc1ccc2c(c1)sc1nc(-c3ccc(NC(=O)Nc4cc(C(C)(C)C)on4)cc3)cn12]

| Formula No. | L | n |
|---|---|---|
| 1097 | 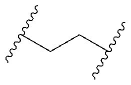 [SMILES: *CC*] | — |
| 1098 | 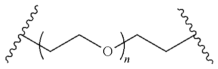 | 1 |
| 1099 | 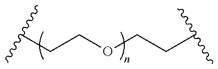 | 2 |
| 1100 | 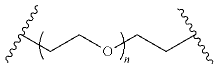 | 3 |
| 1101 | 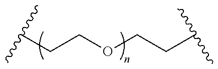 | 4 |
| 1102 | 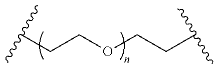 | 5 |

B is

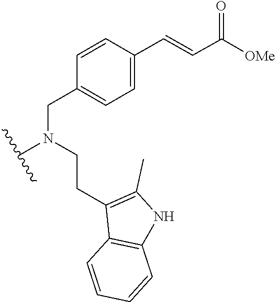 [SMILES: *N(CCc1c(C)[nH]c2ccccc12)Cc1ccc(/C=C/C(=O)OC)cc1]

| Formula No. | L | n |
|---|---|---|
| 1103 | 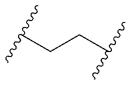 [SMILES: *CC*] | — |
| 1104 | 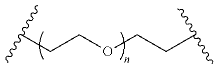 | 1 |
| 1105 | 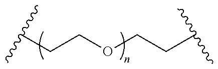 | 2 |
| 1106 | 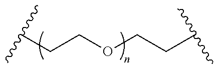 | 3 |
| 1107 | 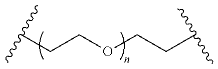 | 4 |
| 1108 | 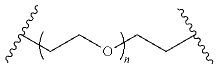 | 5 |

B is

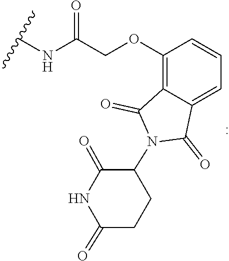 [SMILES: *NC(=O)COc1cccc2c1C(=O)N(C1CCC(=O)NC1=O)C2=O]

| Formula No. | L | n |
|---|---|---|
| 1109 | 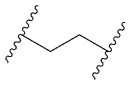 [SMILES: *CC*] | — |
| 1110 | 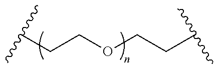 | 1 |
| 1111 | 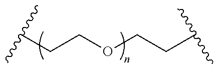 | 2 |
| 1112 | 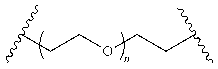 | 3 |
| 1113 | 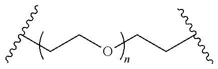 | 4 |
| 1114 | 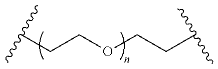 | 5 |

B is

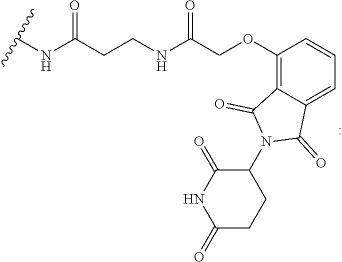 [SMILES: *NC(=O)CCNC(=O)COc1cccc2c1C(=O)N(C1CCC(=O)NC1=O)C2=O]

| Formula No. | L | n |
|---|---|---|
| 1115 | 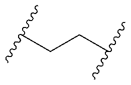 [SMILES: *CC*] | — |
| 1116 | 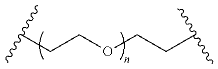 | 1 |
| 1117 | 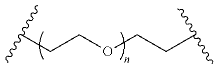 | 2 |
| 1118 | 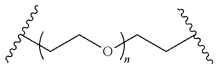 | 3 |
| 1119 | 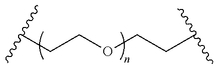 | 4 |
| 1120 | 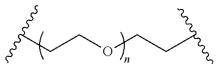 | 5 |

B is

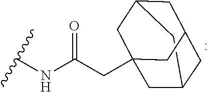 [SMILES: *NC(=O)CC12CC3CC(CC(C3)C1)C2]

| Formula No. | L | n |
|---|---|---|
| 1121 | 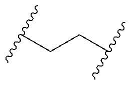 [SMILES: *CC*] | — |
| 1122 | 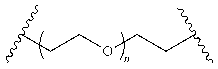 | 1 |
| 1123 | 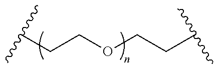 | 2 |
| 1124 | 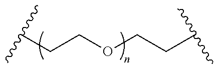 | 3 |
| 1125 | 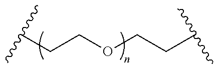 | 4 |
| 1126 | 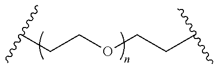 | 5 |

B is

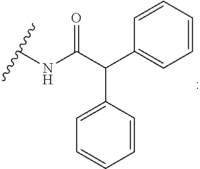 [SMILES: *NC(=O)C(c1ccccc1)c1ccccc1]

| Formula No. | L | n |
|---|---|---|
| 1127 | 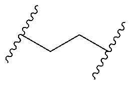 [SMILES: *CC*] | — |
| 1128 | 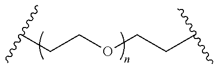 | 1 |
| 1129 | 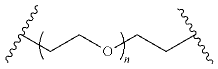 | 2 |
| 1130 | 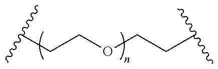 | 3 |
| 1131 | 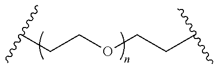 | 4 |
| 1132 | 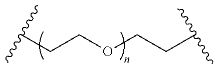 | 5 |

B is

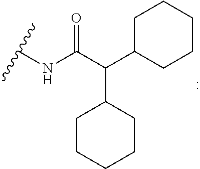 [SMILES: *NC(=O)C(C1CCCCC1)C1CCCCC1]

| Formula No. | L | n |
|---|---|---|
| 1133 | 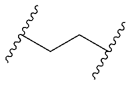 [SMILES: *CC*] | — |
| 1134 | 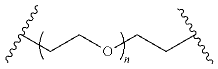 | 1 |
| 1135 | 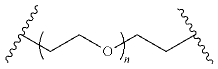 | 2 |
| 1136 | 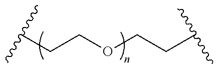 | 3 |
| 1137 | 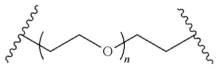 | 4 |
| 1138 | 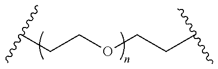 | 5 |

B is

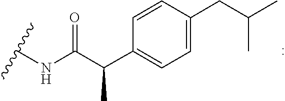 [SMILES: *NC(=O)[C@H](C)c1ccc(CC(C)C)cc1]

| Formula No. | L | n |
|---|---|---|
| 1139 | 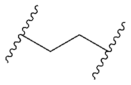 [SMILES: *CC*] | — |
| 1140 | 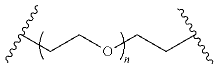 | 1 |
| 1141 | 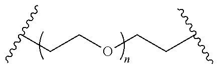 | 2 |
| 1142 | 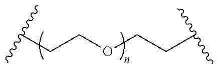 | 3 |
| 1143 | 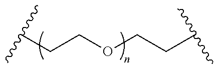 | 4 |
| 1144 | 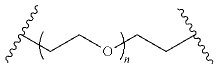 | 5 |

B is

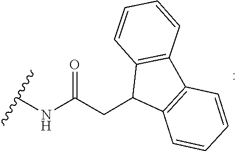 [SMILES: *NC(=O)CC1c2ccccc2-c2ccccc21]

| Formula No. | L | n |
|---|---|---|
| 1145 | 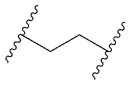 [SMILES: *CC*] | — |
| 1146 | 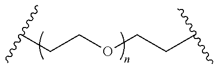 | 1 |
| 1147 | 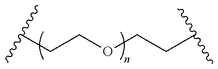 | 2 |
| 1148 | 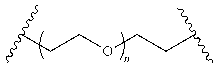 | 3 |
| 1149 | 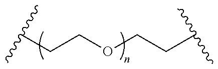 | 4 |
| 1150 | 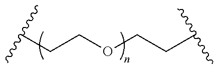 | 5 |

B is

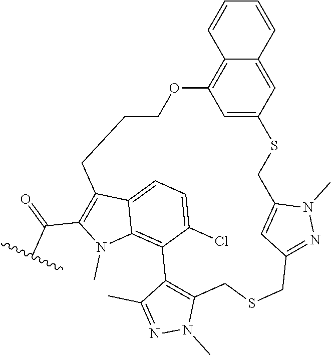 [SMILES: *C(=O)c1c2c3ccc(Cl)c(c3n1C)-c1c(C)nn(C)c1CSCc1cc(n(C)n1)CSc1cc(c3ccccc3c1)OCCC2]

| Formula No. | L | n |
|---|---|---|
| 1151 | 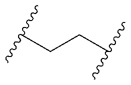 [SMILES: *CC*] | — |
| 1152 | 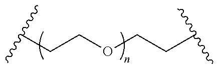 | 1 |
| 1153 | 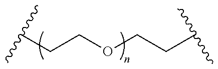 | 2 |
| 1154 | 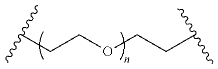 | 3 |
| 1155 | 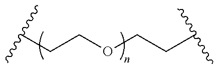 | 4 |
| 1156 | 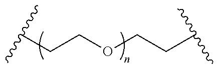 | 5 |

B is

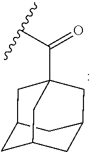 [SMILES: *C(=O)C12CC3CC(CC(C3)C1)C2]

| Formula | | |
|---|---|---|
| No. | L | n |
| 1157 | 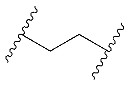 [SMILES: *CC*] | — |
| 1158 | 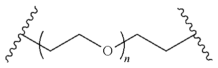 | 1 |
| 1159 | 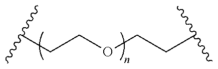 | 2 |
| 1160 | 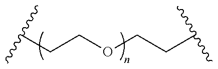 | 3 |
| 1161 | 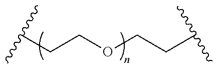 | 4 |
| 1162 | 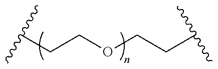 | 5 |

In some embodiments, the compound of formula (110) is a compound of any one of formula 1163-1170, or a pharmaceutically acceptable salt, solvate, hydrate, cocrystal, or prodrug thereof:

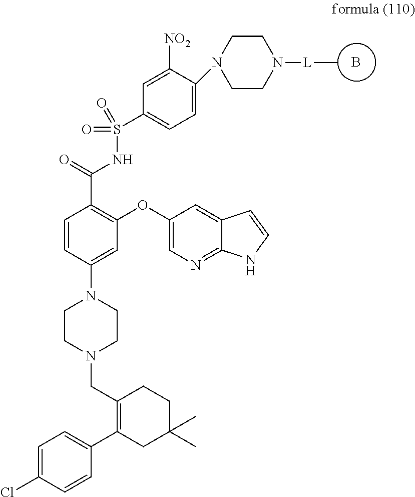

B is

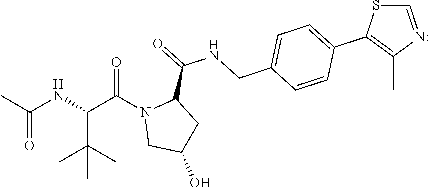

| Formula | | |
|---|---|---|
| No. | L | n |
| 1163 | 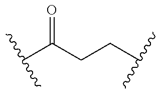 [SMILES: *CCC(*)=O] | — |
| 1164 | 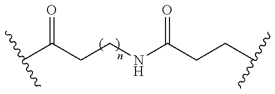 | 0 |
| 1165 | 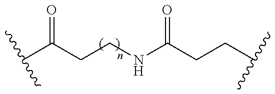 | 1 |
| 1166 | 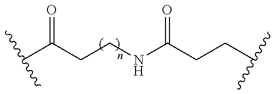 | 2 |
| 1167 | 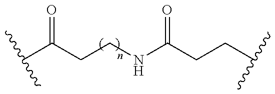 | 3 |
| 1168 | 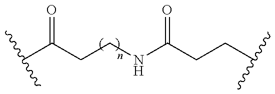 | 4 |
| 1169 | 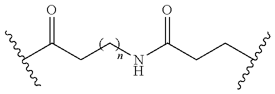 | 5 |
| 1170 | 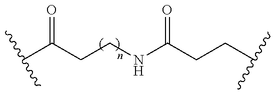 | 6 |

In some embodiments, the compound of formula (I), formula (10), formula (100), formula (110), or formula 1001-1170 has a molecular weight not greater than about 2000 g/mol, or about 1900 g/mol, or about 1800 g/mol, or about 1700 g/mol, or about 1600 g/mol, or about 1500 g/mol, or about 1400 g/mol, or about 1300 g/mol, or about 1200 g/mol or about 1100 g/mol, or about 1000 g/mol.

### Methods of Treatment

The compounds and compositions described herein can be used in methods for treating diseases and disorders. In some embodiments, the compounds and compositions described herein can be used in methods for treating diseases associated with the upregulation of the B-Cell Lymphoma-2 (Bcl-2) protein. In some embodiments, the compounds and compositions described herein can be used in methods for treating diseases associated with the upregulation of myeloid cell leukemia-1 (Mcl-1) oncoprotein. In some embodiments, the compounds and compositions described herein can be used for the treatment of hyperproliferative disorders, including those hyperproliferative disorders associated with the upregulation of Bcl-2 and/or the upregulation of Mcl-1. The compounds and compositions described herein may also be used in treating other disorders as described herein and in the following paragraphs.

In one aspect, the disclosure provides a method of treating or preventing a disease or disorder alleviated by inhibiting Mcl-1 protein activity in a patient in need of said treatment or prevention. In some embodiments, the method comprises administering a therapeutically effective amount of one or more compounds of the disclosure, or a pharmaceutically acceptable salt thereof.

In one aspect, the disclosure provides a method of treating or preventing a disease or disorder alleviated by inhibiting Bcl-2 protein activity in a patient in need of said treatment or prevention. In some embodiments, the method comprises administering a therapeutically effective amount of one or more compounds of the disclosure, or a pharmaceutically acceptable salt thereof.

In one aspect, the disclosure provides a method of treating or preventing a disease or disorder alleviated by inhibiting and/or downregulating Mcl-1 protein activity in a patient in need of said treatment or prevention. In some embodiments, the method comprises administering a therapeutically effective amount of one or more compounds of the disclosure, or a pharmaceutically acceptable salt thereof.

In one aspect, the disclosure provides a method of treating or preventing a disease or disorder alleviated by inhibiting Bcl-2 protein activity and/or inhibiting and/or downregulating Mcl-1 protein activity in a patient in need of said treatment or prevention. In some embodiments, the method comprises administering a therapeutically effective amount of one or more compounds of the disclosure, or a pharmaceutically acceptable salt thereof.

In some embodiments, the Bcl-2 protein activity is inhibited and/or the Mcl-1 protein activity is inhibited and/or downregulated by the compounds of the disclosure binding to a target that inhibits Bcl-1 protein activity and/or downregulates and/or inhibits Mcl-1 protein activity. In some embodiments, the compounds bind to and/or inhibit one or more of CDK9, MEK1/2, HDAC, FLT3, MDM2, JAK1/2, STAT3, ERK1/2, and PI3K/mTOR.

In some embodiments, the disease or disorder is cancer. In some embodiments, the cancer is selected from the cancer is selected from acute myeloid leukemia (AML), chronic lymphocytic leukemia (CLL), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like. In some embodiments, the cancer is acute myeloid leukemia (AML). In some embodiments, the cancer is chronic lymphocytic leukemia (CLL).

In some embodiments, the cancer is a blood cancer. In some embodiments, the blood cancer is selected from acute myeloid leukemia (AML), chronic lymphocytic leukemia (CLL), chronic myeloid leukemia (CML), acute lymphocytic lymphoma (ALL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

In one aspect, the disclosure provides a method of treating or preventing acute myeloid leukemia (AML) in a patient in need of said treatment or prevention. In some embodiments, the method comprises administering a therapeutically effective amount of one or more compounds of the disclosure, or a pharmaceutically acceptable salt thereof.

In one aspect, the disclosure provides a method of treating or preventing chronic lymphocytic leukemia (CLL) in a patient in need of said treatment or prevention. In some embodiments, the method comprises administering a therapeutically effective amount of one or more compounds of the disclosure, or a pharmaceutically acceptable salt thereof.

In some embodiments, the hyperproliferative disorder treated by the compounds and compositions described herein includes cells having Mcl-1 protein and/or Mcl-1 related protein expression. In some embodiments, the disease treated by the compounds and compositions described herein is selected from the group consisting of myeloid leukemia, non-small cell lung cancer, pancreatic cancer, prostate cancer, and ovarian cancer.

In some embodiments, the compounds described herein may induce cell cycle arrest and/or apoptosis in cells containing functional Mcl-1 proteins. The compounds described herein may be used for sensitizing cells to additional agent(s), such as inducers of apoptosis and/or cell cycle arrest, and chemoprotection of normal cells through the induction of cell cycle arrest prior to treatment with chemotherapeutic agents.

In some embodiments, the compounds described herein may be useful for the treatment of disorders, such as those responsive to induction of apoptotic cell death, e.g., disorders characterized by dysregulation of apoptosis. In some embodiments, the compounds may be used to treat cancer that is characterized by resistance to cancer therapies (e.g., those cancer cells which are chemoresistant, radiation resistant, hormone resistant, and the like). In other embodiments, the compounds can be used to treat hyperproliferative diseases characterized by expression of functional Mcl-1 and/or Mcl-1 related proteins, which may or may not be resilient to Bcl-x_{L} inhibitors.

Efficacy of the compounds and combinations of compounds described herein treating the indicated diseases or disorders can be tested using various models known in the art, and described herein, which provide guidance for treatment of human disease.

### Pharmaceutical Compositions

In an embodiment, an active pharmaceutical ingredient or combination of active pharmaceutical ingredients, such as any of the compounds of formula (I), formula 10, or formula 1001-1170, is provided as a pharmaceutically acceptable composition.

In some embodiments, the concentration of each of the active pharmaceutical ingredients provided in the pharmaceutical compositions of the disclosure, such as any of the compounds of formula (I), formula (10), formula (100), formula (110), or formula 1001-1170, is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of each of the active pharmaceutical ingredients provided in the pharmaceutical compositions of the disclosure, such as any of the compounds of formula (I), formula (10), formula (100), formula (110), or formula 1001-1170, is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each of the active pharmaceutical ingredients provided in the pharmaceutical compositions of the disclosure, such as any of the compounds of formula (I), formula (10), formula (100), formula (110), or formula 1001-1170, is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of each of the active pharmaceutical ingredients provided in the pharmaceutical compositions of the disclosure, such as any of the compounds of formula (I), formula (10), formula (100), formula (110), or formula 1001-1170, is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of each of the active pharmaceutical ingredients provided in the pharmaceutical compositions of the disclosure, such as any of the compounds of formula (I), formula (10), formula (100), formula (110), or formula 1001-1170, is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of each of the active pharmaceutical ingredients provided in the pharmaceutical compositions of the disclosure, such as any of the compounds of formula (I), formula (10), formula (100), formula (110), or formula 1001-1170, is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the active pharmaceutical ingredients according to the disclosure is effective over a wide dosage range. For example, in the treatment of adult humans, dosages independently range from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician. The clinically-established dosages of the compounds of formula (I), formula (10), formula (100), formula (110), or formula 1001-1170, may also be used if appropriate.

In an embodiment, the molar ratio of two active pharmaceutical ingredients in the pharmaceutical compositions is in the range from 10:1 to 1:10, preferably from 2.5:1 to 1:2.5, and more preferably about 1:1. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20.

In one aspect, the disclosure provides a pharmaceutical composition comprising one or more of compounds of any one of formula (I), formula (10), formula 1001-1170, or a pharmaceutically acceptable salt thereof, and a physiologically compatible carrier medium.

In one aspect, the disclosure provides a pharmaceutical composition for treating or preventing a disease or disorder alleviated by inhibiting Bcl-2 protein activity, the pharmaceutical composition comprising one or more compounds according to any one of formula (I), formula (10), formula 1001-1170, or a pharmaceutically acceptable salt thereof, and a physiologically compatible carrier medium. In some embodiments, the disease or disorder is cancer.

In one aspect, the disclosure provides a pharmaceutical composition for treating or preventing a disease or disorder alleviated by inhibiting and/or downregulating Mcl-1 protein activity, the pharmaceutical composition comprising one or more compounds according to any one of formula (I), formula (10), formula 1001-1170, or a pharmaceutically acceptable salt thereof, and a physiologically compatible carrier medium. In some embodiments, the disease or disorder is cancer.

In one aspect, the disclosure provides a pharmaceutical composition for treating or preventing a disease or disorder alleviated by both inhibiting Bcl-2 protein activity and inhibiting and/or downregulating Mcl-1 protein activity, the pharmaceutical composition comprising one or more compounds according to any one of formula (I), formula (10), formula (100), formula (110), formula 1001-1170, or a pharmaceutically acceptable salt thereof, and a physiologically compatible carrier medium. In some embodiments, the disease or disorder is cancer.

In some embodiments, the cancer is selected from acute myeloid leukemia (AML), chronic lymphocytic leukemia (CLL), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is acute myeloid leukemia (AML). In some embodiments, the cancer is chronic lymphocytic leukemia (CLL).

In some embodiments, the cancer is a blood cancer. In some embodiments, the blood cancer is selected from acute myeloid leukemia (AML), chronic lymphocytic leukemia (CLL), chronic myeloid leukemia (CML), acute lymphocytic lymphoma (ALL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

Furthermore, F the described methods of treatment may normally include medical follow-up to determine the therapeutic or prophylactic effect brought about in the subject undergoing treatment with the compound(s) and/or composition(s) described herein.

In one aspect, the disclosure provides a pharmaceutical composition for treating or preventing from acute myeloid leukemia (AML), the pharmaceutical composition comprising one or more compounds of formula (I), formula (10), formula (100), formula (110), formula 1001-1170, or a pharmaceutically acceptable salt thereof, and a physiologically compatible carrier medium.

In one aspect, the disclosure provides a pharmaceutical composition for treating or preventing from chronic lymphocytic leukemia (CLL), the pharmaceutical composition comprising one or more compounds of formula (I), formula (10), formula (100), formula (110), formula 1001-1170, or a pharmaceutically acceptable salt thereof, and a physiologically compatible carrier medium.

Described below are non-limiting pharmaceutical compositions and methods for preparing the same.

### Pharmaceutical Compositions for Oral Administration

In an embodiment, the disclosure provides a pharmaceutical composition for oral administration containing the active pharmaceutical ingredient or combination of active pharmaceutical ingredients, such as one or more compounds of formula (I), formula (10), formula (100), formula (110), formula 1001-1170, or a pharmaceutically acceptable salt thereof, and a pharmaceutical excipient suitable for oral administration.

In some embodiments, the disclosure provides a solid pharmaceutical composition for oral administration containing: (i) an effective amount of an active pharmaceutical ingredient or combination of active pharmaceutical ingredients, and (ii) a pharmaceutical excipient suitable for oral administration. In selected embodiments, the composition further contains (iii) an effective amount of a third active pharmaceutical ingredient and optionally (iv) an effective amount of a fourth active pharmaceutical ingredient.

In some embodiments, the pharmaceutical composition may be a liquid pharmaceutical composition suitable for oral consumption. Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogs thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In some embodiments, a pharmaceutical composition is provided for injection containing an active pharmaceutical ingredient or combination of active pharmaceutical ingredients, such as one or more compounds of formula (I), formula (10), formula (100), formula (110), or formula 1001-1170, or a pharmaceutically acceptable salt thereof, and a pharmaceutical excipient suitable for injection.

The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating an active pharmaceutical ingredient or combination of active pharmaceutical ingredients in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In some embodiments, a pharmaceutical composition is provided for transdermal delivery containing an active pharmaceutical ingredient or combination of active pharmaceutical ingredients, such as compounds of formula (I), formula (10), formula (100), formula (110), or formula 1001-1170, and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of an active pharmaceutical ingredient or combination of active pharmaceutical ingredients in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139, the entirety of which are incorporated herein by reference. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra and the compounds of formula (I), formula (10), formula (100), formula (110), and formula 1001-1170 described herein. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions comprising one or more compounds of formula (I), formula (10), formula (100), formula (110), and formula 1001-1170 may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of an active pharmaceutical ingredient or combination of active pharmaceutical ingredients or a pharmaceutical composition thereof can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The active pharmaceutical ingredient or combination of active pharmaceutical ingredients can also be administered intraadiposally or intrathecally.

Exemplary parenteral administration forms include solutions or suspensions of active compound in sterile aqueous solutions, for example, aqueous propylene glycol or dextrose solutions. Such dosage forms can be suitably buffered, if desired.

The disclosure also provides kits. The kits include an active pharmaceutical ingredient or combination of active pharmaceutical ingredients, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, an active pharmaceutical ingredient or combination of active pharmaceutical ingredients are provided as separate compositions in separate containers within the kit. In selected embodiments, an active pharmaceutical ingredient or combination of active pharmaceutical ingredients are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

In some embodiments, the disclosure provides a kit comprising a composition comprising a therapeutically effective amount of an active pharmaceutical ingredient or combination of active pharmaceutical ingredients or a pharmaceutically acceptable salt, solvate, hydrate, cocrystal, or prodrug thereof. These compositions are typically pharmaceutical compositions. The kit is for co-administration of the active pharmaceutical ingredient or combination of active pharmaceutical ingredients, either simultaneously or separately.

In some embodiments, the disclosure provides a kit comprising (1) a composition comprising a therapeutically effective amount of an active pharmaceutical ingredient or combination of active pharmaceutical ingredients or a pharmaceutically acceptable salt, solvate, hydrate, cocrystal, or prodrug thereof, and (2) a diagnostic test for determining whether a patient's cancer is a particular subtype of a cancer. Any of the foregoing diagnostic methods may be utilized in the kit.

The kits described above are preferably for use in the treatment of the diseases and conditions described herein. In a particular embodiment, the kits are for use in the treatment of hyperproliferative disorders.

In a particular embodiment, the kits described herein are for use in the treatment of cancer. In some embodiments, the kits described herein are for use in the treatment of a cancer selected from acute myeloid leukemia (AML), chronic lymphocytic leukemia (CLL), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In particular embodiments, the kits described herein are for use in the treatment of acute myeloid leukemia (AML). In particular embodiments, the kits described herein are for use in the treatment of chronic lymphocytic leukemia (CLL).

### Dosages and Dosing Regimens

The amounts of the pharmaceutical compositions administered using the methods herein, such as the dosages of compounds of formula (I), formula (10), formula (100), formula (110), and formula 1001-1170, will be dependent on the human or mammal being treated, the severity of the disorder or condition, the rate of administration, the disposition of the active pharmaceutical ingredients and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of the pharmaceutical compositions and active pharmaceutical ingredients may be provided in units of mg/kg of body mass or in mg/m² of body surface area.

In some embodiments, the pharmaceutical composition comprising one or more compounds of formula (I), formula (10), formula (100), formula (110), and formula 1001-1170 is administered in combination with one or more anti-cancer agents. Non-limiting examples of anti-cancer agents include abarelix, aldesleukin, alemtuzumab, alitretinoin, allopurinol, altretamine, amifostine, anakinra, anastrozole, arsenic trioxide, asparaginase, azacitidine, bevacizumab, bexarotene, bleomycin, bortezombi, busulfan, calusterone, capecitabine, carmustine, celecoxib, cetuximab, cladribine, cyclophosphamide, cytarabine, carmustine, celecoxib, cetuximab, cladribine, cyclophosphamide, cytarabine, dacarbazine, dactinomycin, actinomycin, dateparin, darbepoetin, dasatinib, daunomycin, decitabine, denileukin, diftitox, dexrazoxane, docetaxel, doxorubicin, dromostanolone, eculizumab, epirubicin, epoetin, erlotinib, estramustine, etoposide, exemestane, fentanyl, filgrastim, floxuridine, 5-FU, fulvestrant, gefitinib, gemcitabine, gem tuzumab, ozogamicin, geldanamycin, goserelin, histrelin, hydroxyurea, ibritumomab, tiuxetan, idarubicin, ifosfamide, imatinib, irinotecan, lapatinib, lenalidomide, letrozole, leucovorin, leuprolide, levamisole, lomustine, CCNU, meclorethamine, megestrol, melphalan, L-PAM, mercaptopurine, 6-MP, mesna, methotrexate, mitomycin C, mitotane, mitoxantrone, nadrolone, nelarabine, nofetumomab, oprelvekin, pegasparagase, pegfilgrastim, peginterferon alpha-2b, pemetrexed, pentostatin, pipobrman, plicamycin, mithramycin, porfimer, procarbazine, quinacrine, rasburicase, rituximab, sargramostim, sorafenib, streptozocin, sunitinib, talc, tamoxifen, temozolomide, teniposide, VM-26, testolactone, thalidomide, thioguanine, 6-thioguanine, thiotepa, topotecan, toremifene, tositumomab, trastuzumab, tretinoin, ATRA, Uracil Mustard, valrubicin, vinorelbine, vorinostat, zoledronate, zoledronic acid, and analogues thereof. In some embodiments, the pharmaceutical composition comprising one or more compounds of formula (I), formula (10), formula (100), formula (110), and formula 1001-1170 is administered prior to, concurrently with, and/or after the administration of one or more anti-cancer agents.

In some embodiments, a pharmaceutical composition or active pharmaceutical ingredient is administered in a single dose. Such administration may be by injection, e.g., intravenous injection, in order to introduce the active pharmaceutical ingredient quickly. However, other routes, including the preferred oral route, may be used as appropriate. A single dose of a pharmaceutical composition may also be used for treatment of an acute condition.

In some embodiments, a pharmaceutical composition or active pharmaceutical ingredient is administered in multiple doses. In an embodiment, a pharmaceutical composition is administered in multiple doses. Dosing may be once, twice, three times, four times, five times, six times, or more than six times per day. Dosing may be once a month, once every two weeks, once a week, or once every other day. In other embodiments, a pharmaceutical composition is administered about once per day to about 6 times per day. In some embodiments, a pharmaceutical composition is administered once daily, while in other embodiments, a pharmaceutical composition is administered twice daily, and in other embodiments a pharmaceutical composition is administered three times daily.

Administration of the active pharmaceutical ingredients may continue as long as necessary. In selected embodiments, a pharmaceutical composition is administered for more than 1, 2, 3, 4, 5, 6, 7, 14, or 28 days. In some embodiments, a pharmaceutical composition is administered for less than 28, 14, 7, 6, 5, 4, 3, 2, or 1 day. In some embodiments, a pharmaceutical composition is administered chronically on an ongoing basis—e.g., for the treatment of chronic effects. In some embodiments, the administration of a pharmaceutical composition continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of an active pharmaceutical ingredient disclosed herein is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of an active pharmaceutical ingredient disclosed herein is about 25 mg, about 50 mg, about 75 mg, about 100 mg, about 125 mg, about 150 mg, about 175 mg, about 200 mg, about 225 mg, or about 250 mg.

In some embodiments, an effective dosage of an active pharmaceutical ingredient disclosed herein is in the range of about 0.01 mg/kg to about 200 mg/kg, or about 0.1 to 100 mg/kg, or about 1 to 50 mg/kg.

In some embodiments, an active pharmaceutical ingredient is adminstered at a dosage of 10 to 200 mg BID, including 50, 60, 70, 80, 90, 100, 150, or 200 mg BID. In some embodiments, an active pharmaceutical ingredient is adminstered at a dosage of 10 to 500 mg BID, including 1, 5, 10, 15, 25, 50, 75, 100, 150, 200, 300, 400, or 500 mg BID.

In some instances, dosage levels below the lower limit of the aforesaid ranges may be more than adequate, while in other cases still larger doses may be employed without causing any harmful side effect—e.g., by dividing such larger doses into several small doses for administration throughout the day. Of course, as those skilled in the art will appreciate, the dosage actually administered will depend upon the condition being treated, the age, health and weight of the recipient, the type of concurrent treatment, if any, and the frequency of treatment. Moreover, the effective dosage amount may be determined by one skilled in the art on the basis of routine empirical activity testing to measure the bioactivity of the compound(s) in a bioassay, and thus establish the appropriate dosage to be administered.

An effective amount of the combination of the active pharmaceutical ingredient may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

In some embodiments, the compositions described herein further include controlled-release, sustained release, or extended-release therapeutic dosage forms for administration of the compounds described herein, which involves incorporation of the compounds into a suitable delivery system in the formation of certain compositions. This dosage form controls release of the compound(s) in such a manner that an effective concentration of the compound(s) in the bloodstream may be maintained over an extended period of time, with the concentration in the blood remaining relatively constant, to improve therapeutic results and/or minimize side effects. Additionally, a controlled-release system would provide minimum peak to trough fluctuations in blood plasma levels of the compound.

The following examples describe the disclosure in further detail. These examples are provided for illustrative purposes only, and should in no way be considered as limiting the disclosure.

### EXAMPLES

### Example 1: PROTAC and Hybrid Compounds for Targeting BCL-2

This Example describes methods to synthetically modify venetoclax in order to directly target two of the most significant mechanisms of venetoclax resistance: MCL-1 upregulation and BCL-2 mutation.

MCL-1 upregulation: Since combination therapy with VEN plus MCL-1 inhibitors may have narrow or no therapeutic window, the solvent-exposed tetrahydropyran moiety of VEN is equipped with additional pharmacophores of drugs known to recognize additional proteins (e.g. FLT3) associated with AML pathogenesis. This is designed to result in a chimeric molecule that not only binds an additional direct targets but also either directly or indirectly downregulates MCL-1, inhibits MCL-1 or upregulates pro-apoptotic BCL-2 proteins. Such rationally-designed polypharmacology is gaining traction in drug development, since inhibiting multiple targets with a single chimeric molecule may yield greater therapeutic efficacies while offering several potential advantages over the more traditional drug cocktail polypharmacy approach, including reduced treatment complexity, reduced drug side effects, reduced drug-drug interactions, reduced pharmacokinetic complexity, and greater patient compliance. In addition, lower doses may be adequate for full efficacy through only partial modulation, rather than abrogation, of targets, thereby resulting in reduced side effects and larger therapeutic windows. This is especially significant for MCL-1 inhibition, as many normal cell types require MCL-1 for survival and in light of the possible cardiotoxicity of an MCL-1 inhibitor in clinical trials; a dual BCL-2/MCL-1 inhibitor may mitigate the toxicity of combining a BCL-2 inhibitor and an MCL-1 inhibitor.

Detection of a variety of BCL-2 mutants in VEN-resistant cells that bind VEN less tightly, yet still in the nM range (as opposed to pM for wild-type BCL-2), indicates that multiple new BCL-2 inhibitors would be needed to effectively inhibit each mutant, which could take years of research or fail. Targeted protein degradation is an emerging field of research in which a modified ligand induces localized unfolding or recruits its target protein to the proteasome for degradation. The latter sub-type, termed proteolysis targeting chimera (PROTAC) technology, involves the tethering of a ligand of the protein of interest (POI) to an E3 ligase ligand, and the resulting polypharmacolgical, bivalent compound ultimately delivers the POI to the proteasome for destruction. Crucially, pM binding affinities are not required, and thus the reduced binding affinities of VEN to the BCL-2 mutants may be adequate. In other words, a BCL-2 PROTAC is expected to be effective at degrading drug-resistant BCL-2. Accordingly, the same tetrahydropyran motif of modified VEN modified is modified with a large, adamantane hydrophobic tag to promote localized unfolding of BCL-2, with subsequent recruitment of HSP40 and HSP70 chaperones, poly-ubiquitination and destruction by the proteasome. Additionally, the grafting of E3 ligase ligands onto this region of VEN is expected to deliver unprecedented BCL-2 PROTACs.

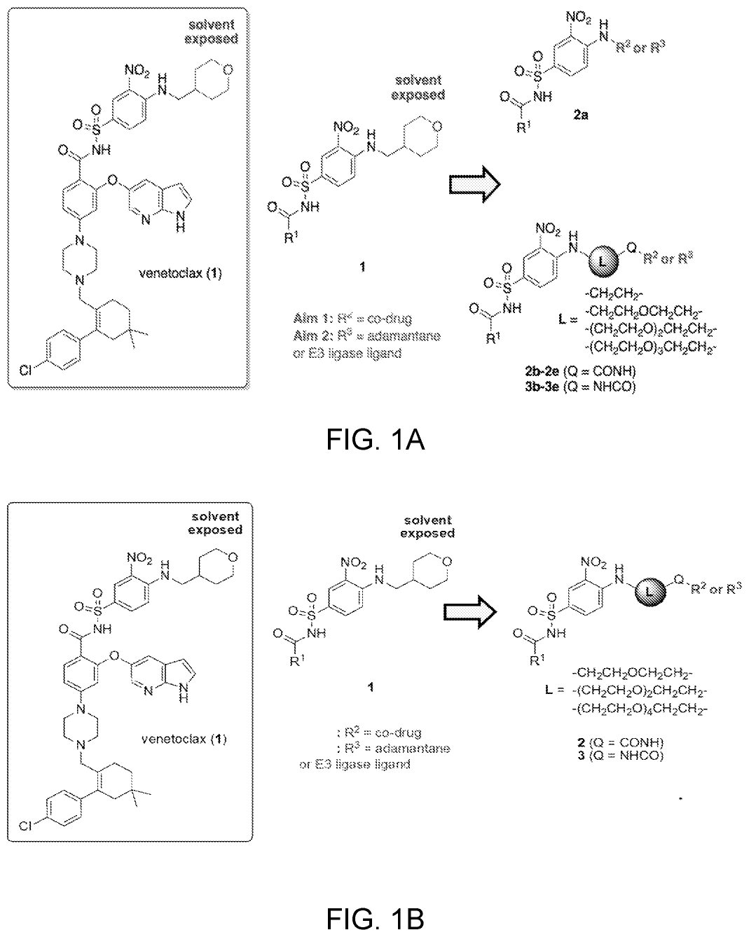

The crystal structure of VEN (1) bound to BCL-2 reveals that the tetrahydropyran motif (blue in FIG. 1A and FIG. 1B) is located near the p4 pocket but crucially is solvent-exposed, suggesting that this portion of the drug may be modifiable with minimal detriment to BCL-2 binding. Indeed, Zheng et al. replaced this moiety with a SAHA-like alkyl hydroxamic acid, and potent BCL-2 (and HDAC) recognition was retained. Accordingly, as illustrated in FIG. 1A and FIG. 1B, the tetrahydropyran moiety of VEN is substituted with (a) secondary drugs (R² groups) to afford dual BCL-2/co-protein inhibitors, and (b) protein degradation tags (R³ groups: adamantane or E3 ligase recognition ligands). The solubilizing basic tertiary amine and the acidic acyl sulfonamide of VEN are retained. It is hypothesized that these second-generation VEN chimeras more potently inhibit the growth of both VEN-sensitive and VEN-resistant AML cells than VEN itself. Moreover, it is hypothesized that the dual inhibitors with secondary drugs are at least as potent in both types of cells as the combination of the corresponding two parent drugs in vitro, and as good, but better, in vivo based on the polypharmacology rationale discussed herein. For each class of secondary drug dual inhibitors, three compounds are prepared with increasing linker length L as depicted in FIG. 1A and FIG. 1B. Q represents an amide or inverse-amide, depending on whether the R² group presents an amine or carboxylic acid for conjugation to VEN. For the protein degradation tag PROTACs, three compounds of each class are prepared, again varying the linker length L as shown. It should be noted that, while these dual inhibitors exhibit high molecular weights (MW), all, except the dual BCL-2/MCL-1 inhibitor, fall well below the MW of the recently-published BCL-XL PROTAC DT2216 (MW=1540) that demonstrates efficacy in murine cancer models.

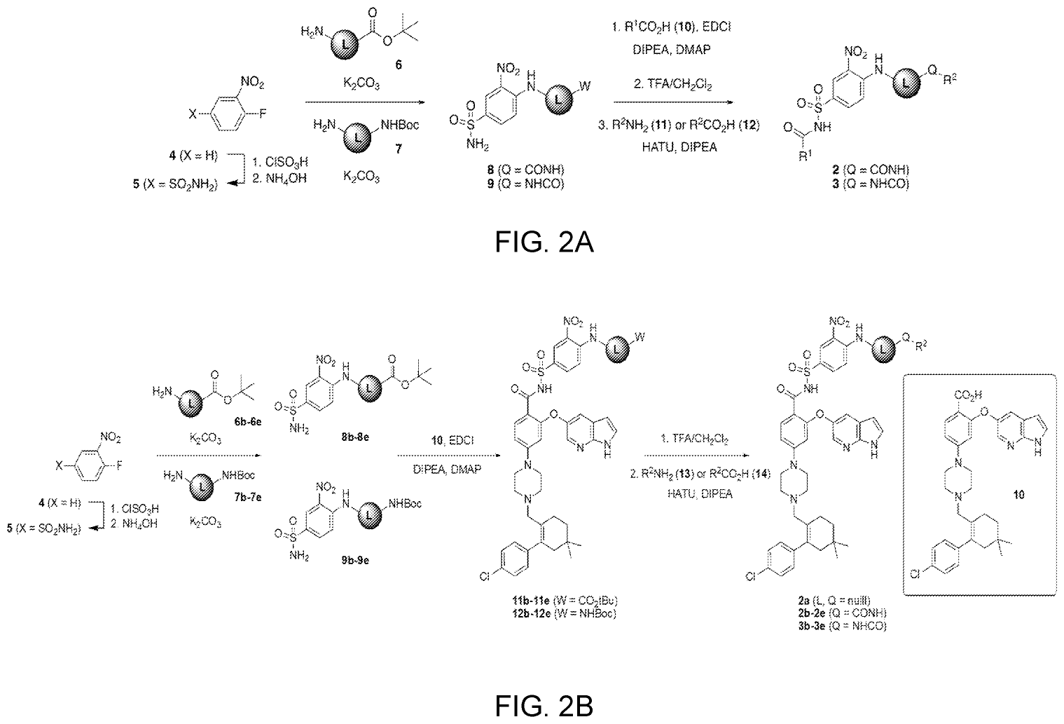

The conjugation chemistry for both secondary drug dual inhibitors and protein degradation tag PROTACs is similar (FIG. 2A and FIG. 2B). In FIG. 2A, 2-fluoronitrobenzene (4) is regioselectively chlorosulfonylated and then quenched with cold ammonium hydroxide to deliver sulfonamide (5). For the amino-terminated R² drugs, sulfonamide 5 is coupled to various length tert-butyl ester-protected amino acids 6 to deliver compounds 8. Subsequently, the primary sulfonamides of 8 are coupled to acid 10, followed by a standard acidic deprotection of the tert-butyl ester with TFA followed by HATU-mediated coupling to drugs R²R²NH/R²NH₂ (11) to yield dual inhibitors 2. For the carboxy-terminated drugs, mono-Boc-protected amino linkers 7 are coupled to sulfonamide 5 to furnish compounds 9. Coupling to acid 10, followed by Boc deprotection and then a final coupling to drugs R²CO₂H (12) provides dual inhibitors 3. Compounds 8 and 9 are also be utilized to generate the BCL-2 PROTACs.

In FIG. 2B, 2-fluoronitrobenzene (4) is regioselectively chlorosulfonylated and then quenched with cold ammonium hydroxide to deliver sulfonamide 5. For the amino-terminated R² drugs, sulfonamide 5 is coupled to various length tert-butyl ester-protected amino acids 6b-6e to deliver compounds 8b-8e. Subsequently, the primary sulfonamides of 8b-8e are coupled to acid 10 to afford compounds 11b-e. Standard acidic deprotection of the tert-butyl ester with TFA followed by HATU-mediated coupling with drugs R²NH₂ (13) yields dual inhibitors 2b-2e. For the carboxy-terminated drugs, mono-Boc-protected amino linkers 7b-7e are coupled to sulfonamide 5 to furnish compounds 9b-9e. Coupling to acid 10, followed by Boc deprotection and then a final coupling to drugs R²CO₂H (14) provides dual inhibitors 3b-3e. Compounds 11b-11e and 12b-12e are also be utilized to generate the BCL-2 PROTACs.

The chemistry in FIG. 2B and FIG. 2B is (a) very straightforward, (b) only six linear steps, and (c) common to both Aims 1 and 2. Indeed, the synthetic burden of this R²¹ is actually minimal: compounds 11 are common precursors to four classes of dual inhibitors and one class of PROTACs, while compounds 12 are common precursors to the remaining two classes of dual inhibitors and the remaining compounds. Only the final step, the HATU coupling, determines if a molecule is dual inhibitor or PROTAC compound, so there is a substantial amount of overlap, mitigating the synthetic chemistry burden. Furthermore, incorporation of the R² drugs in the final step of the syntheses minimizes the amount of R² drugs required, mitigating costs.

Graft Additional Pharmacophores onto VEN to Furnish Dual Inhibitors

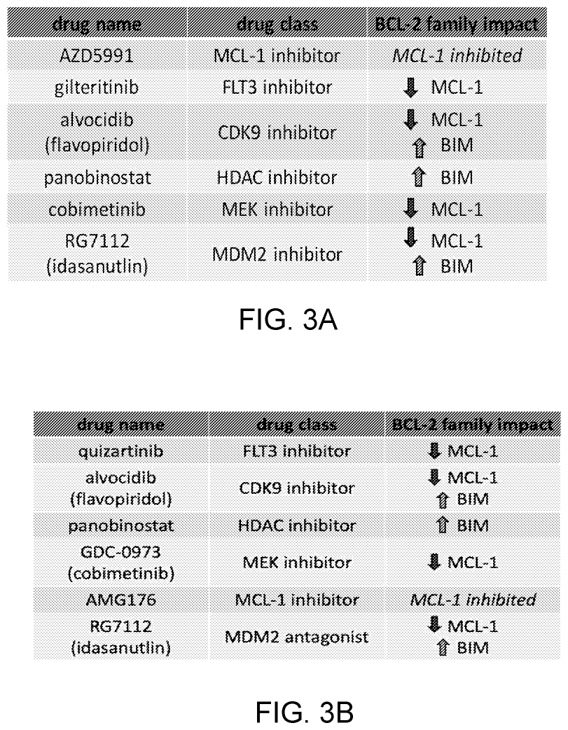

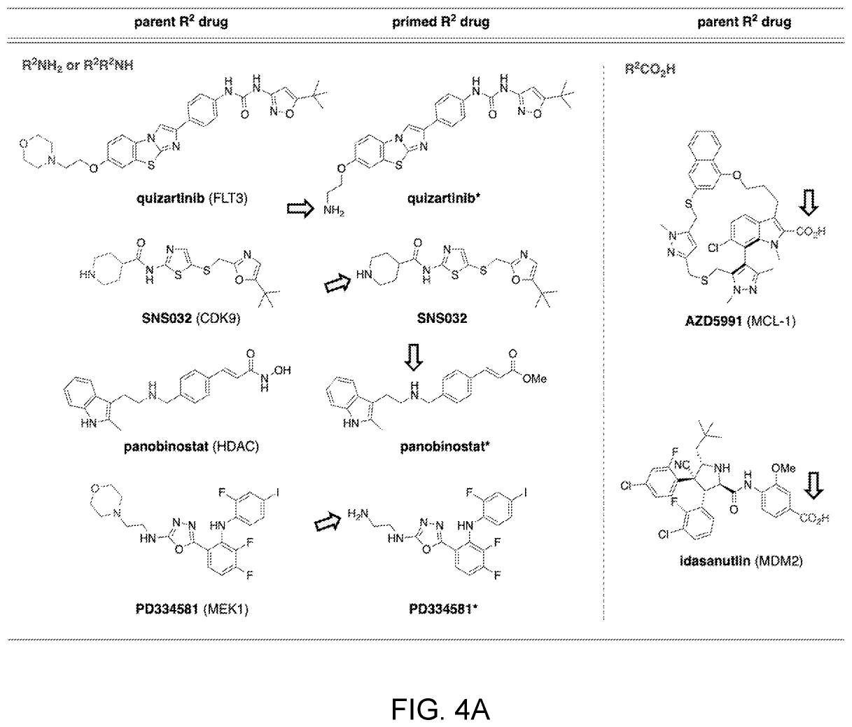

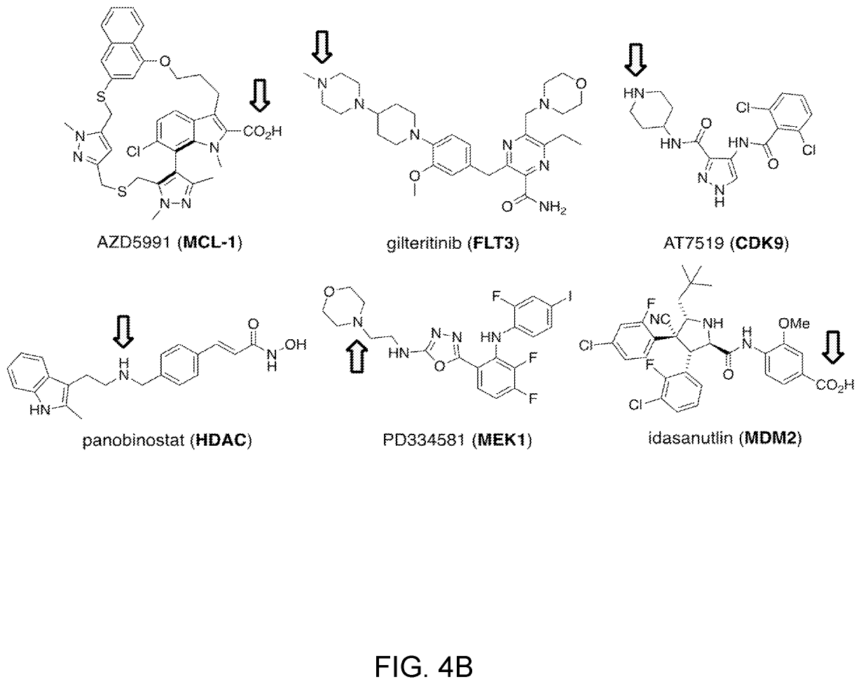

MCL-1 upregulation is a major resistance mechanism to VEN in AML. FIG. 3A and FIG. 3B illustrate drugs that have been co-administered with VEN with synergism in preclinical AML models, which includes the direct MCL-1 inhibitor AZD5991. Inhibition of FLT3, MEK, CDK9 a transcriptional regulator of MCL-1, and MDM2 all result in downregulation of MCL-1. Furthermore, upregulation of pro-apoptotic proteins is observed with inhibition of HDACs, CDK9 and MDM2. In this polypharmacology approach, pre-clinical candidates or clinical drugs targeting these proteins are conjugated to VEN through solvent-exposed regions to ensure minimal impact on protein binding of the parent drug components. Although in certain instances there may be more potent or more selective inhibitors that have emerged recently, the drugs in FIG. 4A and FIG. 4B were selected based on a variety of criteria, including, but not limited to, the availability of co-crystal structures, conjugatable amines or carboxylic acids (or the ability to introduce them), successful clinical trials, reported synergy with VEN, commercial availability and/or ease of synthesis, and molecular weight. Again, a polypharmacology strategy that addresses MCL-1-mediated VEN-resistance is expected to lead to a wider therapeutic window than for the polypharmacy combination of VEN plus an MCL-1 inhibitor, by virtue of anticipated synergy through the co-delivery of both pharmacophores to the same tissue/cells.

In FIG. 4A, the inhibitors have been separated into two classes, based on whether or not they carry an amino group or a carboxylic acid as a point of conjugation with venetoclax.

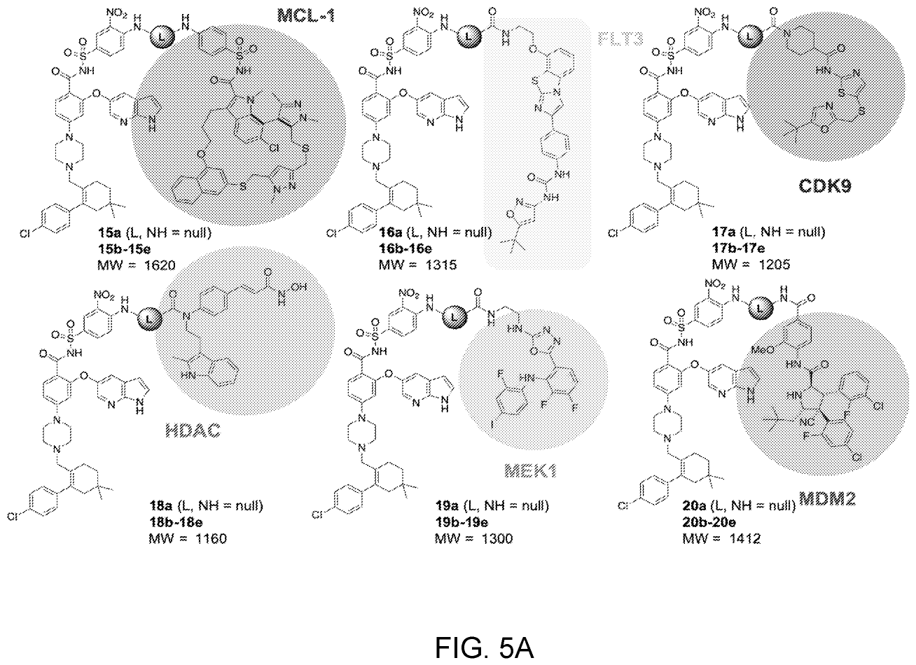

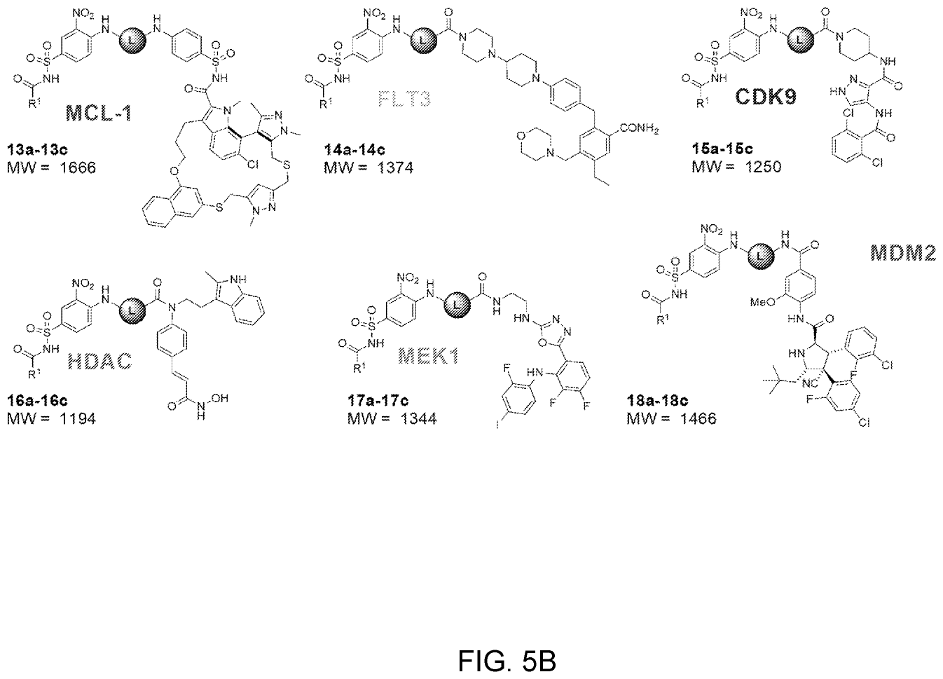

All the drugs showing in FIG. 4A and FIG. 4B are FDA-approved or are in clinical trials. The majority of these inhibitors are commercially available and affordable on the scale required; only two are modified (primed) for this chemistry. The FLT3 inhibitor gilteritinib and the MEK inhibitor PD334581 (close analog PD0325901 is currently enrolled in clinical trials) both possess a solvent-exposed morpholine or piperazine group; these drugs are re-synthesized using established procedures but substituting these moieties with primary amines for conjugation to VEN. Of the amino-based drugs, the CDK9 inhibitor AT7519 (a pan-CDK inhibitor that is most potent against CDK9) and the HDAC inhibitor panobinostat are commerciallyl available affordably; by virtue of their solvent-exposed, secondary amines, they are already primed for our chemistry. In a non-limiting example, if the hydroxamic acid of the pan-HDAC inhibitor panobinostat causes complications during the chemistry, the methyl ester precursor is prepared de novo (3 steps), which is transformed into the requisite hydroxamic acid at the end of the synthesis using standard conditions. The MCL-1 inhibitor AZD5991 (NCT03218683 on-going), and the MDM2 antagonist idasanutlin are available commercially available. Target chimeric drugs are depicted in FIGS. 5A and 5B and are synthesized via the chemistry described in FIG. 2A and/or FIG. 2B. Importantly, the linkage between the MCL-1 inhibitor AZD5991 and VEN is an acyl sulfonamide—this is bioisosteric with carboxylic acids, so retains the crucial acidity of AZD5991 yet also permits linkage to VEN; furthermore, this functional group is found in MCL-1 inhibitors from the Fesik lab and Amgen. Per FIG. 2A and/or FIG. 2B, three compounds spanning different linker lengths of each class of dual inhibitor are prepared, then further evaluated.

### Design and Synthesize Proteolysis-Targeting Chimeras (PROTACs) of VEN

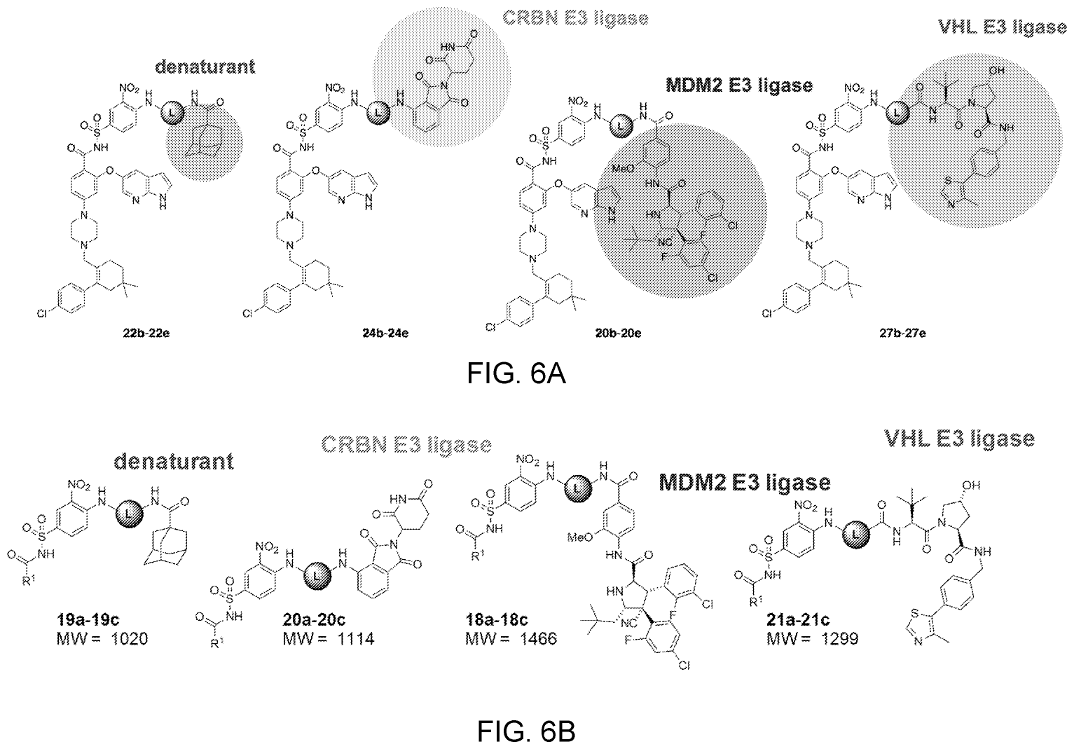

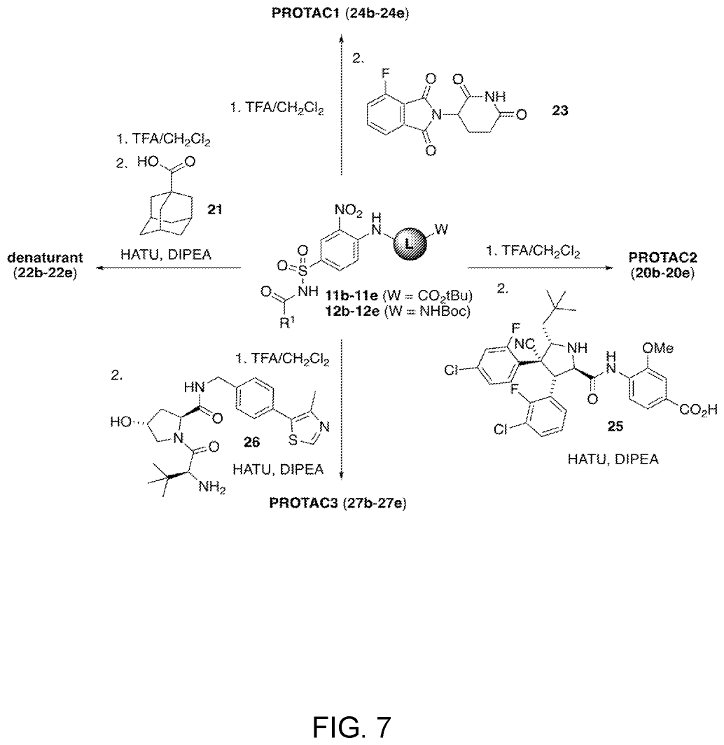

As shown in FIG. 7 and using similar chemistry to that described herein, compounds 12b-12e are Boc-deprotected with TFA, then coupled to adamantane-1-carboxylic acid (21) to deliver denaturants 22b-22e, or undergo a nucleophilic aromatic substitution reaction with aryl fluoride 23 to give PROTAC1 compounds 24b-24e, or coupled to idasanutlin (25) to furnish PROTAC2 compounds (20b-20e (also dual inhibitor compounds)). TFA-mediated removal of the tert-butyl ester of compounds 11b-11e followed by coupling to the VHL ligand 1 (26) furnishes PROTAC3 compounds (27b-27e). As illustrated in FIG. 6A and FIG. 6B, four compounds of each of the denaturant, PROTAC1, PROTAC2 and PROTAC3 compounds are made, varying in only linker length, then further tested. Increasingly longer PEG-linkers were selected based on their use PROTACs and their promotion of solubility.

### Evaluate the Antileukemic Efficacy and Tolerability of VEN Chimeras In Vitro and in Human AML Xenograft Models

In vitro evaluations. All compounds are evaluated in a fluorescence polarization competition assay (FPCA) to determine binding affinities to BCL-2 and the G101V mutant. Those compounds demonstrating Kᵢ values>10-fold weaker than VEN are not be evaluated further; the remainder of the dual inhibitor compounds are evaluated in cell-free competition assays according to their additional pharmacophores: the dual BCL-2/FLT3, BCL-2/CDK9 and BCL-2/MEK inhibitors re evaluated against FLT3, CDK9 and MEK, and compared with the parent R² drugs gilteritinib, AT7519 and PD3304581, respectively; the dual BCL-2/HDAC inhibitors are tested for binding affinities to HDAC1 (Class I) and HDAC6 (Class IIb) utilizing the parent drug panobinostat as the control; the dual BCL-2/MCL-1 and BCL-2/MDM2 inhibitors are analyzed in FPCA assays, with AZD5991 and idasanutlin as respective positive controls. Only those compounds with Kᵢ values<10-fold weaker than the parent R² drugs will be advanced to cell assays.

The IC₅₀s of selected inhibitory compounds are determined by alamarBlue cell viability assays of four established human AML cell lines. Both MV4; 11 and MOLM14 AML cell lines are intrinsically sensitive to VEN in vitro; and harbor a FLT3-ITD mutation plus an MLL rearrangement, both relatively common poor-prognosis AML mutations (both cells lines also contain additional mutations); both cell lines are dependent on MCL-1 and BCL-2, and both express high levels of BCL-2 and MCL-1; both cell lines contain wild type p53 and are sensitive to idasanutlin (MDM2), as well as MEK inhibitors, panobinostat (HDACs) and CDK9 inhibitors. In addition, the human OCI-AML2 and OCI-AML3 cell lines are used, which exhibit acquired and intrinsic resistance, respectively, to VEN. Three repeats of each IC₅₀ determination assay are performed for each compound against each of these four AML cell lines. Multiple compounds are tested against all four AML cell lines at once, using alamarBlue microplate assays previously described. Specifically, cell lines are treated with the vehicle (negative control), VEN alone, the given lead chimeric compound, and then either (dual inhibitor compounds) the parent R² drug (e.g. gilteritinib) alone and the combination of VEN+R² drug, or (PROTACs) an E3 ligase-inert VEN-based PROTAC each at multiple concentrations, and IC₅₀s are determined. Polypharmacologic inhibitors that are at least equipotent to, but ideally better than, the 2-drug combination of VEN+R² drug against the AML cell lines are identified, especially VEN-resistant OCI-AML2 and OCI-AML3, and the PROTACs are expected to be superior to VEN alone.

Co-immunoprecipitation assays are employed with the active PROTACs to determine if a dose-response degradation of BCL-2 is observed. In such cases, the aforementioned cell viability assays are subsequently be conducted.

Prior to their final selection for in vivo testing, results of the alamarBlue assays for the top dual inhibitors are confirmed by Annexin and Caspase assays to quantify apoptosis, since apoptotic cytotoxicity is expected, and, for the PROTACs, Western blotting to determine if dose-dependent degradation of BCL-2 is observed. On-target specific cytotoxicity is confirmed by determining if lentiviral overexpression of BCL-2 (in a non-limiting example, in both MV4; 11 and OCI-AML3) protects the cells from compound-dependent apoptosis in a BCL-2-level-dependent manner, with acknowledgement that BCL-2 rescue is not specific. Finally, to evaluate in vitro hematopoietic toxicity, primary human CD34⁺ hematopoietic stem-progenitor cells from healthy adult donors are exposed to the most potent dual inhibitors and the most potent PROTAC for 48 h (controls: vehicle, VEN, partner drug, etc. as above), then the ability of drug-treated cells to generate hematopoietic colonies is quantitated.

In vivo evaluation of the most potent dual inhibitor and the most potent PROTAC begins with pilot experiments to determine the single-dose maximal tolerated dose (MTD) in NOD/Rag^{KO}/Il2gamma^{KO} (NRG) mice. To determine single-dose MTDs, dose escalation toxicity studies are conducted in non-leukemia-bearing NRG mice administered the novel VEN chimeric drug PO (gavage) and assessed for lethal toxicity within 7d, using a traditional 3+3 design. Tolerable PO daily X5d dosing for each novel compound is determined, starting with 20% of the single-dose MTD as the daily dose in an established 5d schedule. The daily dose is increased or decreased, sequentially as necessary, to find the 5d repeated-dose MTD for each of the two chosen VEN chimeric compounds.

Non-limiting example of in vivo testing: The two selected VEN chimeric compounds are tested at their 5d MTD dose-schedule against a human fluorescent protein (FP)/Luc-labelled MOLM14 AML cell line xenograft model that has been established for antileukemic drug evaluation. NRG mice are infused with Luc-labelled human MOLM14 AML cells on day −10. After Xenogen quantification of baseline total body luminescence on do, mice are placed into experimental groups balanced for AML burden. Each experiment to evaluate the two chosen VEN chimeras in vivo involves 7 groups of 5 MOLM14 AML-bearing mice treated with: (1) vehicle, (2) selected dual inhibitors, (3) selected PROTACs, (4) R² partner drug (e.g. gilteritinib), (5) a synthetic VEN-based PROTAC negative control whose E3 ligase capacity is incapacitated (VEN modified identically as in FIG. 1A or FIG. 1B but with a PROTAC-inactive ligand, e.g. N-methylated thalidomide), (6) VEN, and (7) VEN+R² partner drug. VEN and partner drug are administered at the same molar concentration as the VEN-based chimeras; additional control groups are needed if the MTDs of the two chimeric compounds differ. AML-bearing NRG mice are dosed daily PO on d1-5 and d21-25, assessed for AML burden by Xenogen imaging on d7, d14, and d21, and monitored until clinical signs dictate need for euthanasia (projected average 10 weeks). Primary response endpoints are AML response quantitation (fold-change in AML burden on d7/14/21 vs d0 for each mouse) via imaging. Comparisons of quantitative imaging results are confirmed by Kaplan-Meier survival analyses.

Each of the four selected inhibitors are tested at its 5d MTD dose-schedule against a human fluorescent protein (FP)/Luc-labelled MV4; 11 AML cell line (venetoclax-sensitive) xenograft model has been established for antileukemic drug evaluation. NRG mice are be infused with Luc-labelled human AML cells on day −10. After Xenogen quantification of baseline total body luminescence on do, mice are placed into experimental groups balanced for AML burden prior to drug administration via gavage, then mice are dosed daily PO (gavaged) with drug(s) on a 5-days-on, 9-days-off schedule, until clinical signs dictate need for euthanasia (projected average 10 weeks).

In a non-limiting example, each experiment to evaluate in vivo the four selected second-generation venetoclax derivatives involves 9 groups of 5 mice treated with: (1) vehicle, (2-4) three selected dual inhibitors (A, B and C (in some embodiments, targeting different secondary proteins)), (5) lead PROTAC inhibitor, (6) venetoclax, (7) venetoclax+parental co-drug of selected inhibitor A (e.g. quizartinib), (8) venetoclax+parental co-drug of 1 selected inhibitor B, (9) venetoclax+parental co-drug of selected inhibitor C. Each of the top novel inhibitory compounds are evaluated for in vivo inhibition of first the MV4; 11. A fluorescent protein (FP)/Luc-labelled OCI-AML3 cell line (venetoclax-resistant) is constructed and the drugs are tested similarly in the OCI-AML3 xenograft model. A novel chimeric venetoclax derivative is identified (polypharmacology) that, when tested individually, results in at least comparable reduction of venetoclax-sensitive AML burden and significantly greater reduction of venetoclax-resistant AML burden (by Xenogen imaging of luciferase) on d7 as compared to venetoclax as a monotherapy, and also venetoclax and the relevant parental drug administered together (polypharmacy), relative to the do leukemia burden immediately prior to treatment. The initial response results are confirmed by testing for significantly superior Kaplan-Meier survival for the chimeric venetoclax derivative compared to the same controls. These in vivo experiments are interpreted as pilot experiments, with results to be confirmed statistically in a future project with larger numbers of mice and additional AML xenograft models. Mouse weights are measured weekly and monitor clinical signs, as general indicators of drug toxicity. In some embodiments, blood cell counts and chemistries are assessed in the same models.

Non-limiting example of in vivo testing: The two selected VEN chimeric compounds are tested at their 5d MTD dose-schedule against a human fluorescent protein (FP)/Luc-labelled MOLM14 AML cell line xenograft model that has been established for antileukemic drug evaluation. NRG mice are infused with Luc-labelled human MOLM14 AML cells on day −10. After Xenogen quantification of baseline total body luminescence on do, mice are placed into experimental groups balanced for AML burden. Each experiment to evaluate the two chosen VEN chimeras in vivo involves 7 groups of 5 MOLM14 AML-bearing mice treated with: (1) vehicle, (2) selected dual inhibitor, (3) lead PROTAC (4) R² partner drug (e.g. gilteritinib), (5) a synthetic VEN-based PROTAC negative control whose E3 ligase capacity is incapacitated (VEN modified identically as in FIG. 1A and/or FIG. 1B but with a PROTAC-inactive ligand, e.g. N-methylated thalidomide), (6) VEN, and (7) VEN+R² partner drug. VEN and partner drug are administered at the same molar concentration as the VEN-based chimeras; additional control groups are needed if the MTDs of the two chimeric compounds differ. AML-bearing NRG mice are dosed daily PO on d1-5 and d21-25, assessed for AML burden by Xenogen imaging on d7, d14, and d21, and monitored until clinical signs dictate need for euthanasia (projected average 10 weeks). Primary response endpoints are AML response quantitation (fold-change in AML burden on d7/14/21 vs d0 for each mouse) via imaging. Comparisons of quantitative imaging results are confirmed by Kaplan-Meier survival analyses.

In addition, a fluorescent protein (FP)/Luc-labelled OCI-AML3 cell line is constructed and the two chosen VEN chimeras tested similarly using this xenograft model. Two novel VEN chimeras are identified that each result in vivo in at least comparable reduction of MOLM14 (VEN-sensitive) AML burden and significantly greater reduction of OCI-AML3 (VEN-resistant) AML burden, compared to (a) VEN alone, (b) partner drug alone, and (c) VEN plus the partner drug co-administered.

Therapeutic window: Mouse body weights are measured weekly, as a general indicator of drug toxicity. To assess hematopoietic toxicity in vivo, two additional groups of 5 (non-AML-bearing) NRG mice are treated with each of the two chosen VEN chimeras or the above controls, and on d10 of treatment cycle #2, these mice are bled for complete blood counts (CBCs) and euthanized.

Statistics, sample size: To provide an empiric estimate of needed mice/group, a pilot experiment using a MOLM14 AML xenograft model and two 5d PO treatment cycles was performed (5 mice/group). VEN alone at the established standard dose had no detectable effect vs treatment with vehicle, on either AML burden over time or survival (adjusted p=0.722 at d21 and 1.0 at d84, respectively). Sorafenib (SOR; inhibits FLT3 and other kinases) alone at an established dose, reduced AML burden only moderately (adjusted p=0.091 at d21). Co-administration of VEN plus SOR resulted in deeper, longer AML burden reductions (adjusted p<0.05 at d21) and prolonged survivals (p=0.048 at d84), vs vehicle or either drug alone. If each of the two selected novel VEN chimeras is at least as effective as co-administered VEN plus SOR, 5 mice/group are expected to be sufficient to demonstrate statistical superiority over VEN or the partner alone in reducing AML burden and possibly prolonging survival. Addition of a third drug (either PEGylated L-asparaginase (ASP) or an artemisinin derivative (ART) further reduced AML burden (adjusted p<0.001) and prolonged survival (p<0.01 at d84), compared to vehicle or monotherapy (VEN or SOR). If each of the two selected novel VEN chimeras is at least as effective as co-administered VEN plus SOR plus these third drugs, 5 mice/group is expected to be sufficient to demonstrate statistical superiority over vehicle or monotherapy (VEN or SOR) in prolonging survival. Required mouse numbers are estimated based on inferences from these preliminary results; a similar or better efficacy for the novel compounds is expected, especially against the VEN-resistant OCI-AML3 model. Thus, the proposed sample size (5 mice/group) is expected to provide sufficient power for statistical analyses (at p<0.05 level; α=0.05, β=0.1), which is estimated with the standard deviation of AML burden over time monitored and the expected drug efficacy differences. Moreover, each in vivo experiment is repeated twice (n=10 mice/group total). Statistical analyses are conducted using GraphPad software. AML burden is quantified using log transformed ratio of luminescence at d7/14/21 vs do for each mouse (fold-change); groups are compared using the appropriate parametric (ANOVA; t-test, etc) or non-parametric alternative as necessary (Wilcoxon rank sum, Kruskal-Wallis). For time-to-event endpoints, Kaplan-Meier survival of groups are compared using the log-rank test. p<0.05 is considered statistically different.

Method for Treatment of AML xenografts with venetoclax monotherapy or combinations: NRG mice were transplanted IV with Luc-labelled MOLM14 AML cells. On d0, after Xenogen quantification of baseline total body luminescence (AML burden), groups of 5 AML-bearing mice were treated on days 1-5, per previously established MTD PO×5d schedule, with VEN (150 mg/kg/d PO×5), as monotherapy or co-administered with SOR (30 mg/kg/d PO×5), ART (50 mg/kg/d PO×5), and/or ASP (200 IU/kg IP single dose). A second identical drug cycle was administered on d21-25. On d7, d14 and d21, each mouse was Xenogen imaged for total body luminescence, fold-change AML burden from do was calculated for each mouse, and mean (+/−SD) fold-change for each experimental group plotted. Statistically significant differences (p<0.05) in AML burden were found between vehicle and VEN+SOR, VEN+SOR+ASP, VEN+SOR+ART, respectively, at d7, d14, and d21. SOR monotherapy caused significant difference in AML burden only at d7 and d14, as compared to Vehicle. Kaplan-Meier survivals. Statistically significant differences (p<0.05) in survived animal number were found between the combination treatments and Vehicle or monotherapy (VEN or SOR). Monotherapy with ART or ASP had minimal activity as well, similar to VEN monotherapy and Vehicle.

Chemical syntheses are conducted concurrently, and compounds are evaluated in the cell-free competition assays as they are synthesized. Lead compound scale-up is performed for in vivo work. In vitro and in vivo evaluations of the top novel compounds is conducted.

In grafting R² drugs and R³ PROTAC ligands onto VEN, some solvent-exposed, solubilizing groups are removed. A problem with solubility of the resulting chimeric inhibitors is not expected, owing to the residual two ionizable groups (the basic tertiary amine and acidic acyl sulfonamide) present in the retained portion of VEN. In a non-limiting example, an additional ionizable tertiary amine is introduced into the linker L if necessary. In a non-limiting example, if problems arise with re-synthesis of the co-drugs that need priming (FIG. 4A or FIG. 4B) for conjugation chemistry, or if a loss in activity of the parent drug is observed, the next best drug from that class will be chosen based on the selection criteria described above. In a non-limiting example, if the PROTACs in are ineffective degraders of BCL-2, then longer linkers can be employed, as PROTACs can be sensitive to linker length. In a non-limiting example, if no improvement in cell killing is observed with the chimeric VEN compounds in VEN-resistant cells, this may be due to upregulation of BCL-xL, whose inhibition is undesirable as this has been associated with thrombcytopenia.

### Example 2: BCL-2 Inhibition and Cell Viability Testing

This Example describes BCL-2 inhibition and cell viability testing of select inhibitors.

Fluorescence polarization assays were performed in 96 well polypropylene F-bottom black microplates (Greiner Bio-One) with a final volume of 100 μL. During the competition assay, a fluorescently-labeled Bak-BH3 peptide (FITC-Ahx-GQVGRQLAIIGDDINR-CONH₂ (SEQ ID NO.: 1), hereafter “FITC-Bak”, where FITC=fluorescein isocyanate; Ahx=6-aminohexanoyl linker) was competed off of either MCL-1¹⁷²⁻³²⁷, BCL-X_{L}¹⁻²¹² or BCL-2¹⁻²¹¹ with the synthesized inhibitors. The binding affinities of FITC-Bak to MCL-1, BCL-X_{L} and BCL-2 were determined via a fluorescence polarization assay where various concentrations of the selected proteins were titrated into solutions of 10 nM FITC-Bak in 20 mM HEPES, pH 6.8, 50 mM NaCl, 3 mM DTT, 0.01% Triton X-100 and 5% DMSO at room temperature. The changes in the fluorescence polarization were then measured using a BMG PHERAstar FS multimode microplate reader equipped with two PMTs for simultaneous measurements of both the perpendicular and parallel fluorescence emission at a 485 nm excitation and 520 nm emission filter. Regression analysis was then performed on the polarization data using Origin (OriginLab, Northampton, MA) and the data was fitted to the Hill equation, thus producing binding curves for FITC-Bak with MCL-1, BCL-X_{L} and BCL-2. FITC-Bak's K_{d}'s were then determined to be 42 nM for MCL-1, 6 nM for BCL-X_{L} and 33 nM for BCL-2.

The fluorescence polarization competition assays were setup using a Biomek FXP Automated Liquid Handling System. Protein concentrations of either 100 nM of MCL-1, 15 nM of BCL-X_{L} or 75 nM of BCL-2 with 10 nM of FITC-Bak (in 20 mM HEPES, pH 6.8, 50 mM NaCl, 3 mM DTT, 0.01% Triton X-100 and 1% DMSO) were chosen and various concentrations of the inhibitors were titrated into the solutions. Wells possessing only the peptide, only the desired protein and both the peptide plus the desired protein without inhibitor were used as controls. Changes in fluorescence polarization were measured after 4 hours of incubation at room temperature using the BMG PHERAstar FS multimode plate reader previously mentioned and regression analysis was performed using Prism 8 (Graphpad) with the data fitted to a sigmoidal curve to determine inhibitor IC₅₀ values. The IC₅₀ values were then converted to Kᵢ values using an equation derived by Nikolovska-Coleska et al. All inhibitors were tested in triplicate.

### Testing Cell Viability

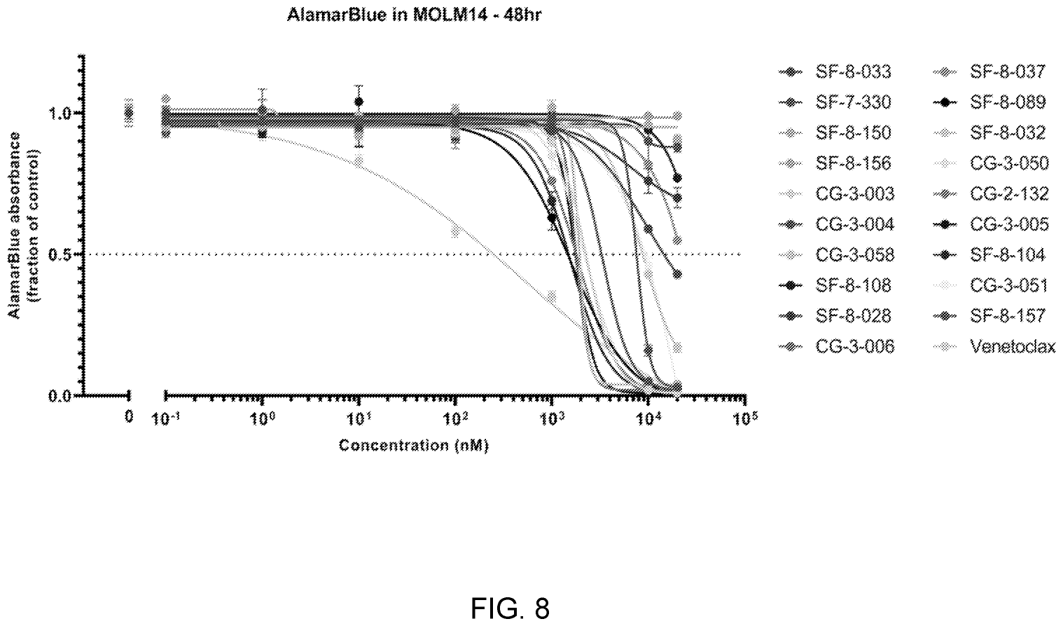

Cell viability of MOLM14 cells was measured in presence of test compounds via alamarBlue assay. This is a colorimetric assay that uses resazurin, a reduction-oxidation indicator. After incubation, resazurin is added to the test wells. The reduced form of resazurin is resorufin, which is pink and highly fluorescent. This can be measured through absorbance or fluorescence and is proportional to the amount of living cells. The compounds tested are presented in Table 1 and the results are presented in FIG. 8.

Modifications to venetoclax by varying the solvent-exposed morpholine group with this series of “SF” and “CG” PROTACs and hydrophobic-tagged (HyT) denaturants is tolerated—all tested compounds retained venetoclax's ability to inhibit BCL-2 (venetoclax Ki in FPCA assay conditions: <20 nM). In cell-killing assays, most of this series likewise retained the activity of the parent drug venetoclax.

TABLE 1
Test compounds used to evaluate viability of MOLM14 cells via alamarBlue assay.

| Com- | | Molecular | BCL-2 |
|---|---|---|---|
| pound | | Weight | IC₅₀ |
| Name | Structure | (g/mol) | (nM) |
| SF-8- 028 | 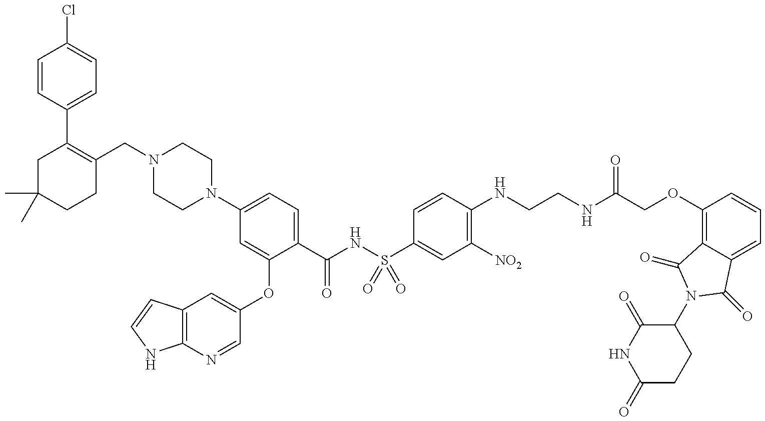 [SMILES: CC1(C)CCC(CN2CCN(c3ccc(C(=O)NS(=O)(=O)c4ccc(NCCNC(=O)COc5cccc6c5C(=O)N(C5CCC(=O)NC5=O)C6=O)c([N+](=O)[O-])c4)c(Oc4cnc5[nH]ccc5c4)c3)CC2)=C(c2ccc(Cl)cc2)C1] | 1127.62 | <20 |
| SF-8- 032 | 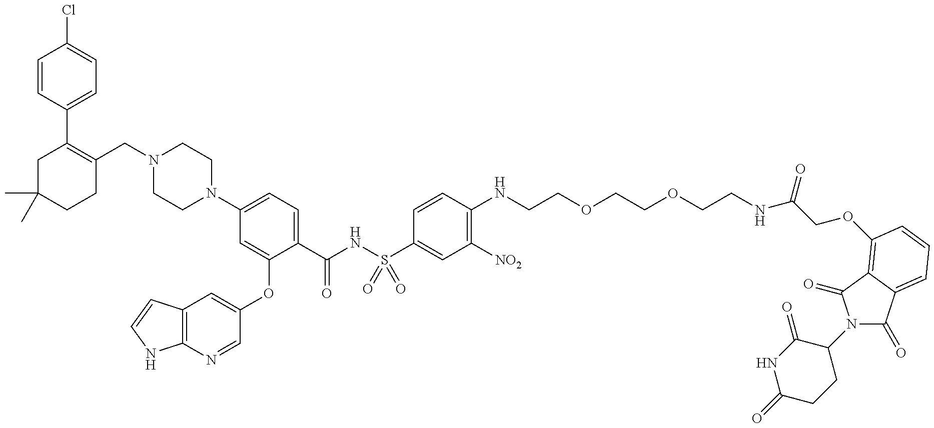 [SMILES: CC1(C)CCC(CN2CCN(c3ccc(C(=O)NS(=O)(=O)c4ccc(NCCOCCOCCNC(=O)COc5cccc6c5C(=O)N(C5CCC(=O)NC5=O)C6=O)c([N+](=O)[O-])c4)c(Oc4cnc5[nH]ccc5c4)c3)CC2)=C(c2ccc(Cl)cc2)C1] | 1215.73 | <20 |
| SF-8- 037 | 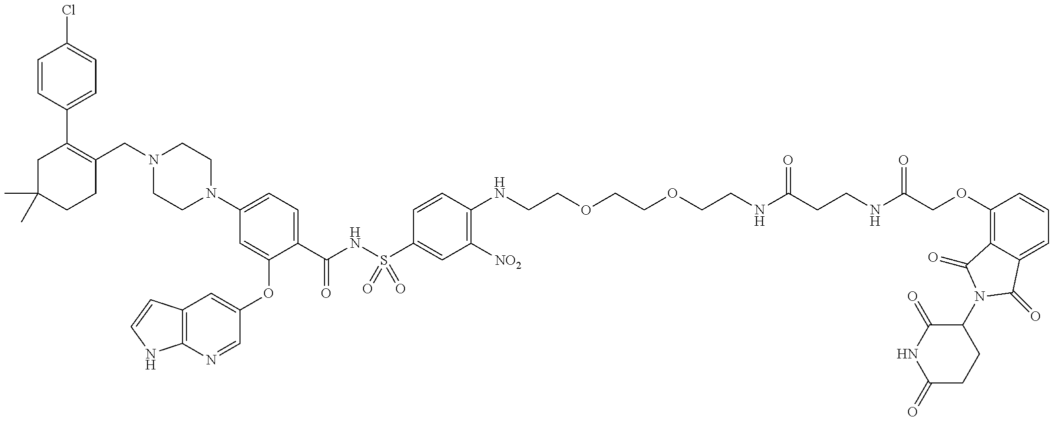 [SMILES: CC1(C)CCC(CN2CCN(c3ccc(C(=O)NS(=O)(=O)c4ccc(NCCOCCOCCNC(=O)CCNC(=O)COc5cccc6c5C(=O)N(C5CCC(=O)NC5=O)C6=O)c([N+](=O)[O-])c4)c(Oc4cnc5[nH]ccc5c4)c3)CC2)=C(c2ccc(Cl)cc2)C1] | 1286.81 | <20 |
| SF-8- 089 | 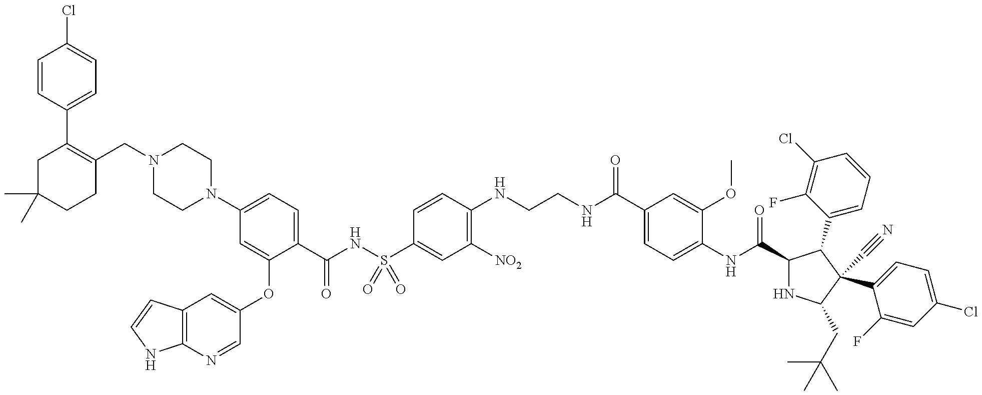 [SMILES: COc1cc(C(=O)NCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])ccc1NC(=O)[C@@H]1N[C@@H](CC(C)(C)C)[C@](C#N)(c2ccc(Cl)cc2F)[C@H]1c1cccc(Cl)c1F] | 1411.84 | <20 |
| SF-8- 104 | 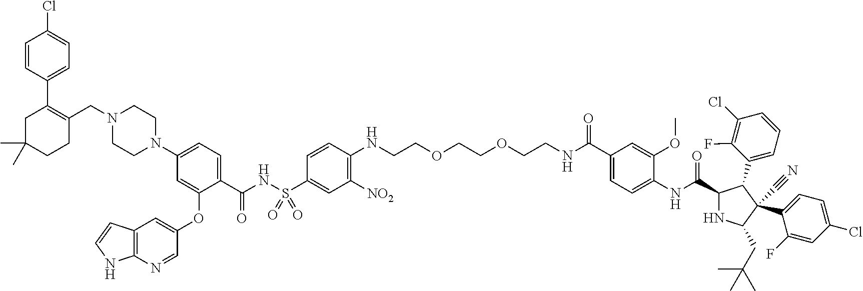 [SMILES: COc1cc(C(=O)NCCOCCOCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])ccc1NC(=O)[C@@H]1N[C@@H](CC(C)(C)C)[C@](C#N)(c2ccc(Cl)cc2F)[C@H]1c1cccc(Cl)c1F] | 1499.95 | <20 |
| SF-8- 150 | 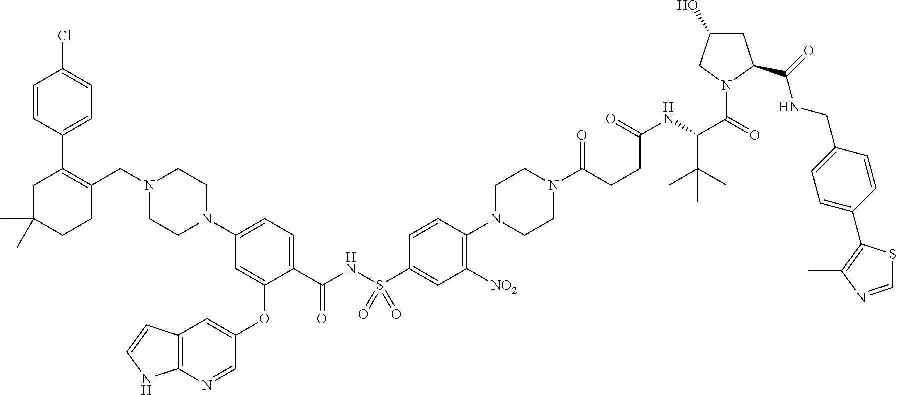 [SMILES: Cc1ncsc1-c1ccc(CNC(=O)[C@@H]2C[C@@H](O)CN2C(=O)[C@@H](NC(=O)CCC(=O)N2CCN(c3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3[N+](=O)[O-])CC2)C(C)(C)C)cc1] | 1352.03 | |
| SF-8- 156 | 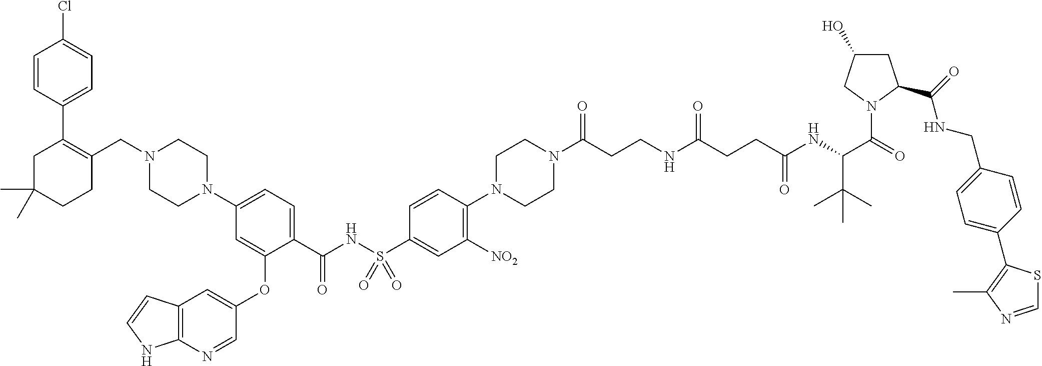 [SMILES: Cc1ncsc1-c1ccc(CNC(=O)[C@@H]2C[C@@H](O)CN2C(=O)[C@@H](NC(=O)CCC(=O)NCCC(=O)N2CCN(c3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3[N+](=O)[O-])CC2)C(C)(C)C)cc1] | 1423.11 | |
| SF-8- 157 | 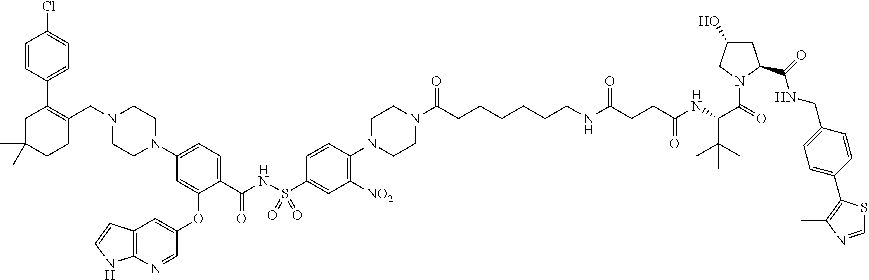 [SMILES: Cc1ncsc1-c1ccc(CNC(=O)[C@@H]2C[C@@H](O)CN2C(=O)[C@@H](NC(=O)CCC(=O)NCCCCCCC(=O)N2CCN(c3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3[N+](=O)[O-])CC2)C(C)(C)C)cc1] | 1479.22 | |
| CG-3- 003 | 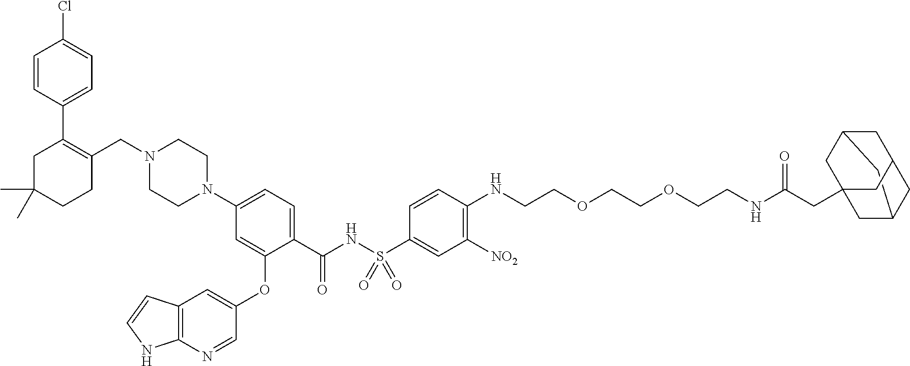 [SMILES: CC1(C)CCC(CN2CCN(c3ccc(C(=O)NS(=O)(=O)c4ccc(NCCOCCOCCNC(=O)CC56CC7CC(CC(C7)C5)C6)c([N+](=O)[O-])c4)c(Oc4cnc5[nH]ccc5c4)c3)CC2)=C(c2ccc(Cl)cc2)C1] | 1077.74 | |
| CG-3- 004 | 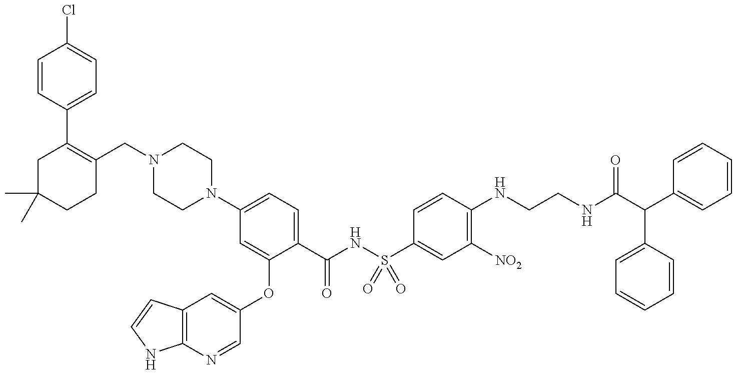 [SMILES: CC1(C)CCC(CN2CCN(c3ccc(C(=O)NS(=O)(=O)c4ccc(NCCNC(=O)C(c5ccccc5)c5ccccc5)c([N+](=O)[O-])c4)c(Oc4cnc5[nH]ccc5c4)c3)CC2)=C(c2ccc(Cl)cc2)C1] | 1007.60 | |
| CG-3- 005 | 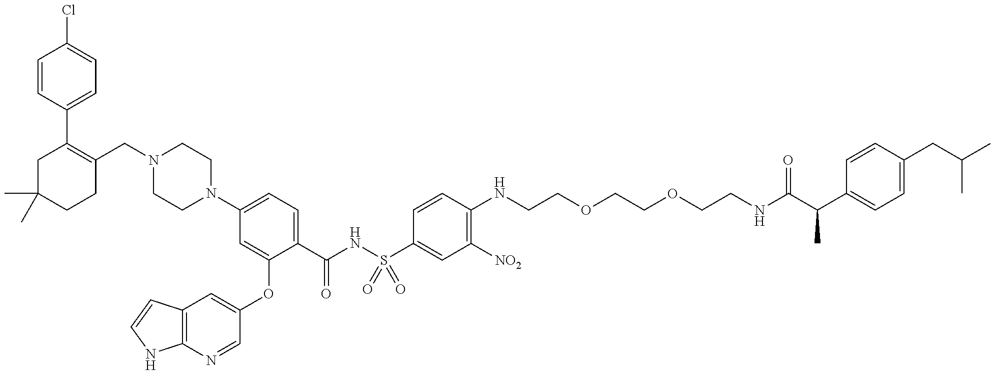 [SMILES: CC(C)Cc1ccc([C@@H](C)C(=O)NCCOCCOCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])cc1] | 1089.75 | <20 |
| SF-7- 330 | 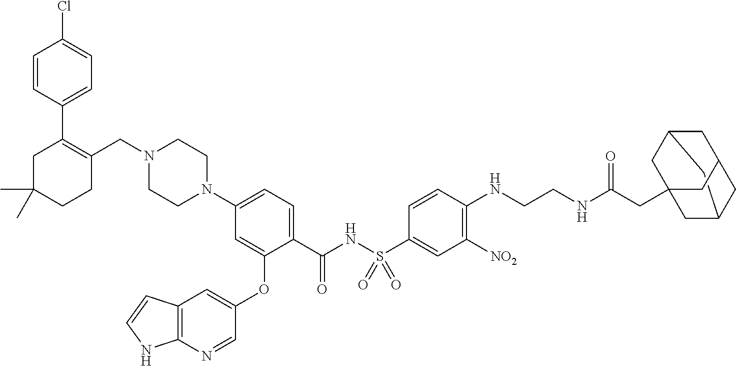 [SMILES: CC1(C)CCC(CN2CCN(c3ccc(C(=O)NS(=O)(=O)c4ccc(NCCNC(=O)CC56CC7CC(CC(C7)C5)C6)c([N+](=O)[O-])c4)c(Oc4cnc5[nH]ccc5c4)c3)CC2)=C(c2ccc(Cl)cc2)C1] | 989.63 | <20 |
| SF-8- 108 | 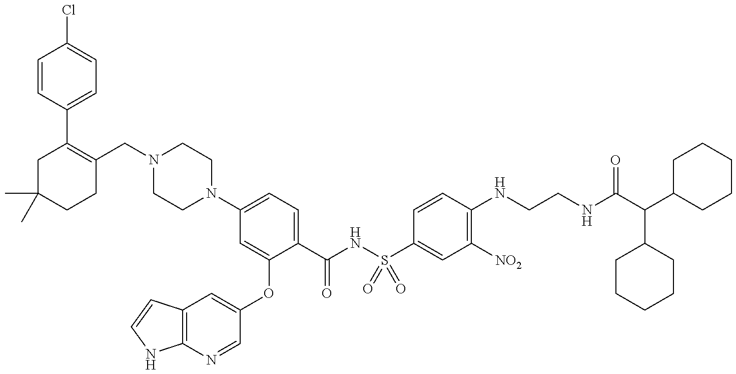 [SMILES: CC1(C)CCC(CN2CCN(c3ccc(C(=O)NS(=O)(=O)c4ccc(NCCNC(=O)C(C5CCCCC5)C5CCCCC5)c([N+](=O)[O-])c4)c(Oc4cnc5[nH]ccc5c4)c3)CC2)=C(c2ccc(Cl)cc2)C1] | 1019.70 | |
| CG-2- 132 | 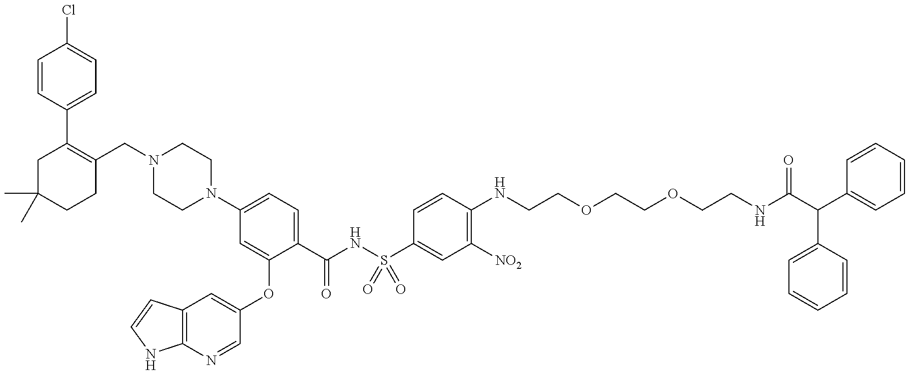 [SMILES: CC1(C)CCC(CN2CCN(c3ccc(C(=O)NS(=O)(=O)c4ccc(NCCOCCOCCNC(=O)C(c5ccccc5)c5ccccc5)c([N+](=O)[O-])c4)c(Oc4cnc5[nH]ccc5c4)c3)CC2)=C(c2ccc(Cl)cc2)C1] | 1095.71 | <20 |
| CG-3- 006 | 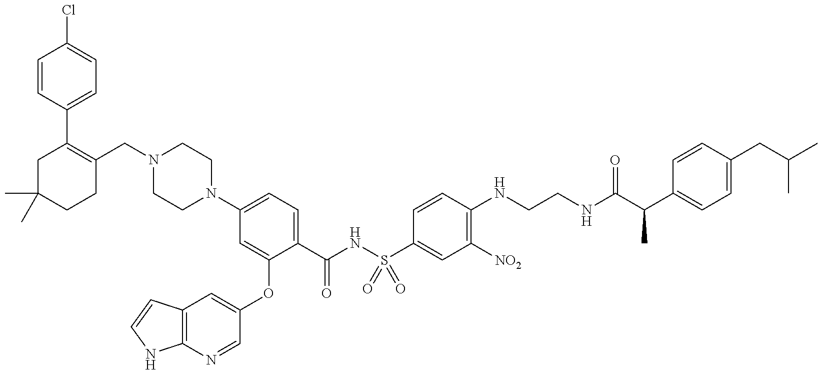 [SMILES: CC(C)Cc1ccc([C@@H](C)C(=O)NCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])cc1] | 1001.64 | |
| CG-3- 050 | 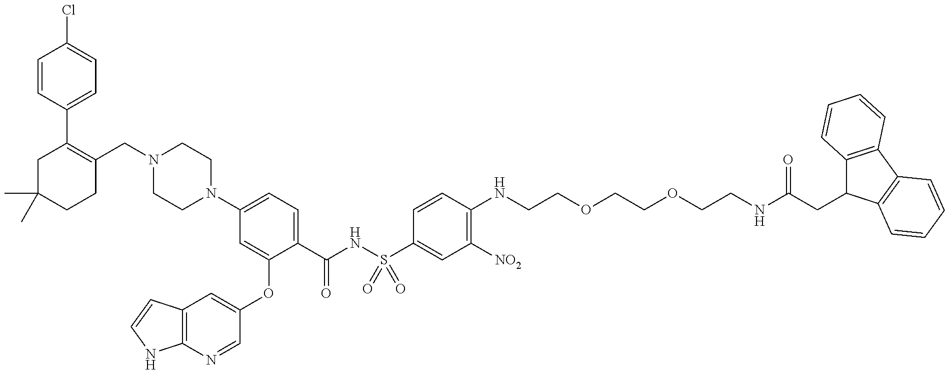 [SMILES: CC1(C)CCC(CN2CCN(c3ccc(C(=O)NS(=O)(=O)c4ccc(NCCOCCOCCNC(=O)CC5c6ccccc6-c6ccccc65)c([N+](=O)[O-])c4)c(Oc4cnc5[nH]ccc5c4)c3)CC2)=C(c2ccc(Cl)cc2)C1] | 1107.72 | |
| CG-3- 051 | 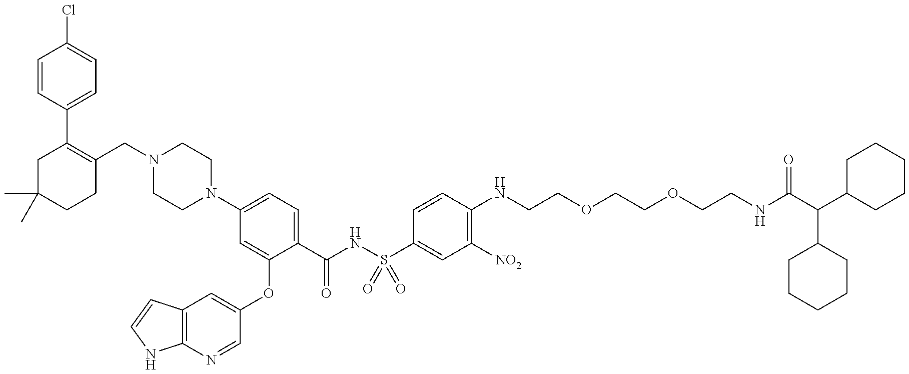 [SMILES: CC1(C)CCC(CN2CCN(c3ccc(C(=O)NS(=O)(=O)c4ccc(NCCOCCOCCNC(=O)C(C5CCCCC5)C5CCCCC5)c([N+](=O)[O-])c4)c(Oc4cnc5[nH]ccc5c4)c3)CC2)=C(c2ccc(Cl)cc2)C1] | 1107.81 | |
| CG-3- 058 | 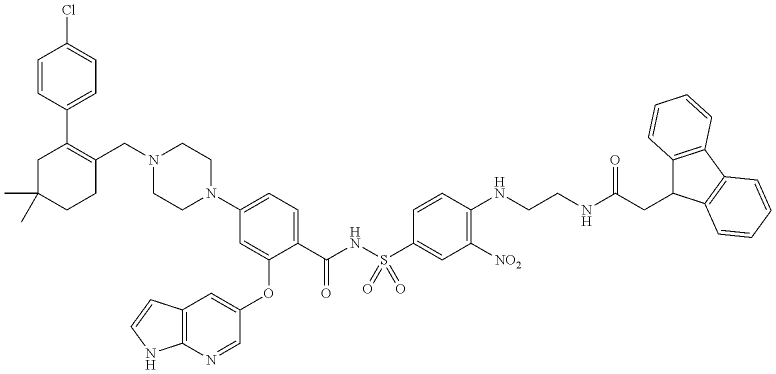 [SMILES: CC1(C)CCC(CN2CCN(c3ccc(C(=O)NS(=O)(=O)c4ccc(NCCNC(=O)CC5c6ccccc6-c6ccccc65)c([N+](=O)[O-])c4)c(Oc4cnc5[nH]ccc5c4)c3)CC2)=C(c2ccc(Cl)cc2)C1] | 1019.62 | |

A number of patent and non-patent publications are cited herein in order to describe the state of the art to which this disclosure pertains. The entire disclosure of each of these publications is incorporated by reference herein.

While certain embodiments of the present disclosure have been described and/or exemplified above, various other embodiments will be apparent to those skilled in the art from the foregoing disclosure. The present disclosure is, therefore, not limited to the particular embodiments described and/or exemplified, but is capable of considerable variation and modification without departure from the scope and spirit of the appended claims.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claims. All compounds, compositions, formulations, and methods described herein that embody the present disclosure can, in alternate embodiments, be more specifically defined by any of the transitional terms “comprising,” “consisting essentially of,” and “consisting of.”

### REFERENCES

1. www.cancer.org/cancer/acute-myeloid-leukemia/about/key-statistics.html.
2. Dohner, H.; Estey, E. H.; Amadori, S.; Appelbaum, F. R.; Buchner, T.; Burnett, A. K.; Dombret, H.; Fenaux, P.; Grimwade, D.; Larson, R. A.; Lo-Coco, F.; Naoe, T.; Niederwieser, D.; Ossenkoppele, G. J.; Sanz, M. A.; Sierra, J.; Tallman, M. S.; Lowenberg, B.; Bloomfield, C. D.; European, L., Diagnosis and management of acute myeloid leukemia in adults: recommendations from an international expert panel, on behalf of the European LeukemiaNet. Blood 2010, 115 (3), 453-74.
3. Oran, B.; Weisdorf, D. J., Survival for older patients with acute myeloid leukemia: a population-based study. Haematologica 2012, 97 (12), 1916-24.
4. DiNardo, C. D.; Pratz, K.; Pullarkat, V.; Jonas, B. A.; Arellano, M.; Becker, P. S.; Frankfurt, O.; Konopleva, M.; Wei, A. H.; Kantarjian, H. M.; Xu, T.; Hong, W. J.; Chyla, B.; Potluri, J.; Pollyea, D. A.; Letai, A., Venetoclax combined with decitabine or azacitidine in treatment-naive, elderly patients with acute myeloid leukemia. Blood 2019, 133 (1), 7-17.
5. DiNardo, C. D.; Pratz, K. W.; Letai, A.; Jonas, B. A.; Wei, A. H.; Thirman, M.; Arellano, M.; Frattini, M. G.; Kantarjian, H.; Popovic, R.; Chyla, B.; Xu, T.; Dunbar, M.; Agarwal, S. K.; Humerickhouse, R.; Mabry, M.; Potluri, J.; Konopleva, M.; Pollyea, D. A., Safety and preliminary efficacy of venetoclax with decitabine or azacitidine in elderly patients with previously untreated acute myeloid leukaemia: a non-randomised, open-label, phase 1b study. Lancet Oncol 2018, 19 (2), 216-228.
6. Kavanagh, S.; Murphy, T.; Law, A.; Yehudai, D.; Ho, J. M.; Chan, S.; Schimmer, A. D., Emerging therapies for acute myeloid leukemia: translating biology into the clinic. JCI Insight 2017, 2 (18).
7. Konopleva, M.; Letai, A., BCL-2 inhibition in AML: an unexpected bonus? Blood 2018, 132 (10), 1007-1012.
8. Lin, K. H.; Winter, P. S.; Xie, A.; Roth, C.; Martz, C. A.; Stein, E. M.; Anderson, G. R.; Tingley, J. P.; Wood, K. C., Targeting MCL-1/BCL-XL Forestalls the Acquisition of Resistance to ABT-199 in Acute Myeloid Leukemia. Sci Rep 2016, 6, 27696.
9. Wang, Q.; Wan, J.; Zhang, W.; Hao, S., MCL-1 or BCL-xL-dependent resistance to the BCL-2 antagonist (ABT-199) can be overcome by specific inhibitor as single agents and in combination with ABT-199 in acute myeloid leukemia cells. Leuk Lymphoma 2019, 60 (9), 2170-2180.
10. Perini, G. F.; Ribeiro, G. N.; Pinto Neto, J. V.; Campos, L. T.; Hamerschlak, N., BCL-2 as therapeutic target for hematological malignancies. J Hematol Oncol 2018, 11 (1), 65.
11. Kale, J.; Osterlund, E. J.; Andrews, D. W., BCL-2 family proteins: changing partners in the dance towards death. Cell Death Differ 2018, 25 (1), 65-80.
12. Adams, J. M.; Cory, S., The BCL-2 arbiters of apoptosis and their growing role as cancer targets. Cell Death Differ 2018, 25 (1), 27-36.
13. Souers, A. J.; Leverson, J. D.; Boghaert, E. R.; Ackler, S. L.; Catron, N. D.; Chen, J.; Dayton, B. D.; Ding, H.; Enschede, S. H.; Fairbrother, W. J.; Huang, D. C.; Hymowitz, S. G.; Jin, S.; Khaw, S. L.; Kovar, P. J.; Lam, L. T.; Lee, J.; Maecker, H. L.; Marsh, K. C.; Mason, K. D.; Mitten, M. J.; Nimmer, P. M.; Oleksijew, A.; Park, C. H.; Park, C. M.; Phillips, D. C.; Roberts, A. W.; Sampath, D.; Seymour, J. F.; Smith, M. L.; Sullivan, G. M.; Tahir, S. K.; Tse, C.; Wendt, M. D.; Xiao, Y.; Xue, J. C.; Zhang, H.; Humerickhouse, R. A.; Rosenberg, S. H.; Elmore, S. W., ABT-199, a potent and selective BCL-2 inhibitor, achieves antitumor activity while sparing platelets. Nat Med 2013, 19 (2), 202-8.
14. Wei, A. H.; Tiong, I. S., Midostaurin, enasidenib, CPX-351, gemtuzumab ozogamicin, and venetoclax bring new hope to AML. Blood 2017, 130 (23), 2469-2474.
15. Yap, J. L.; Chen, L.; Lanning, M. E.; Fletcher, S., Expanding the Cancer Arsenal with Targeted Therapies: Disarmament of the Antiapoptotic Bcl-2 Proteins by Small Molecules. J Med Chem 2017, 60 (3), 821-838.
16. Konopleva, M.; Pollyea, D. A.; Potluri, J.; Chyla, B.; Hogdal, L.; Busman, T.; McKeegan, E.; Salem, A. H.; Zhu, M.; Ricker, J. L.; Blum, W.; DiNardo, C. D.; Kadia, T.; Dunbar, M.; Kirby, R.; Falotico, N.; Leverson, J.; Humerickhouse, R.; Mabry, M.; Stone, R.; Kantarjian, H.; Letai, A., Efficacy and Biological Correlates of Response in a Phase II Study of Venetoclax Monotherapy in Patients with Acute Myelogenous Leukemia. Cancer Discov 2016, 6 (10), 1106-1117.
17. Juarez-Salcedo, L. M.; Desai, V.; Dalia, S., Venetoclax: evidence to date and clinical potential. Drugs Context 2019, 8, 212574.
18. Tahir, S. K.; Smith, M. L.; Hessler, P.; Rapp, L. R.; Idler, K. B.; Park, C. H.; Leverson, J. D.; Lam, L. T., Potential mechanisms of resistance to venetoclax and strategies to circumvent it. BMC Cancer 2017, 17 (1), 399.
19. Konopleva, M.; Milella, M.; Ruvolo, P.; Watts, J. C.; Ricciardi, M. R.; Korchin, B.; McQueen, T.; Bornmann, W.; Tsao, T.; Bergamo, P.; Mak, D. H.; Chen, W.; McCubrey, J.; Tafuri, A.; Andreeff, M., MEK inhibition enhances ABT-737-induced leukemia cell apoptosis via prevention of ERK-activated MCL-1 induction and modulation of MCL-1/BIM complex. Leukemia 2012, 26 (4), 778-87.
20 Ramsey, H. E.; Fischer, M. A.; Lee, T.; Gorska, A. E.; Arrate, M. P.; Fuller, L.; Boyd, K. L.; Strickland, S. A.; Sensintaffar, J.; Hogdal, L. J.; Ayers, G. D.; Olejniczak, E. T.; Fesik, S. W.; Savona, M. R., A Novel MCL1 Inhibitor Combined with Venetoclax Rescues Venetoclax-Resistant Acute Myelogenous Leukemia. Cancer Discov 2018, 8 (12), 1566-1581.
21 Moujalled, D. M.; Pomilio, G.; Ghiurau, C.; Ivey, A.; Salmon, J.; Rijal, S.; Macraild, S.; Zhang, L.; Teh, T. C.; Tiong, I. S.; Lan, P.; Chanrion, M.; Claperon, A.; Rocchetti, F.; Zichi, A.; Kraus-Berthier, L.; Wang, Y.; Halilovic, E.; Morris, E.; Colland, F.; Segal, D.; Huang, D.; Roberts, A. W.; Maragno, A. L.; Lessene, G.; Geneste, O.; Wei, A. H., Combining BH3-mimetics to target both BCL-2 and MCL1 has potent activity in pre-clinical models of acute myeloid leukemia. Leukemia 2019, 33 (4), 905-917.
22. Caenepeel, S.; Brown, S. P.; Belmontes, B.; Moody, G.; Keegan, K. S.; Chui, D.; Whittington, D. A.; Huang, X.; Poppe, L.; Cheng, A. C.; Cardozo, M.; Houze, J.; Li, Y.; Lucas, B.; Paras, N. A.; Wang, X.; Taygerly, J. P.; Vimolratana, M.; Zancanella, M.; Zhu, L.; Cajulis, E.; Osgood, T.; Sun, J.; Damon, L.; Egan, R. K.; Greninger, P.; McClanaghan, J. D.; Gong, J.; Moujalled, D.; Pomilio, G.; Beltran, P.; Benes, C. H.; Roberts, A. W.; Huang, D. C.; Wei, A.; Canon, J.; Coxon, A.; Hughes, P. E., AMG 176, a Selective MCL1 Inhibitor, Is Effective in Hematologic Cancer Models Alone and in Combination with Established Therapies. Cancer Discov 2018, 8 (12), 1582-1597.
23. Opferman, J. T.; Iwasaki, H.; Ong, C. C.; Suh, H.; Mizuno, S.; Akashi, K.; Korsmeyer, S. J., Obligate role of anti-apoptotic MCL-1 in the survival of hematopoietic stem cells. Science 2005, 307 (5712), 1101-4.
24. Wang, X.; Bathina, M.; Lynch, J.; Koss, B.; Calabrese, C.; Frase, S.; Schuetz, J. D.; Rehg, J. E.; Opferman, J. T., Deletion of MCL-1 causes lethal cardiac failure and mitochondrial dysfunction. Genes Dev 2013, 27 (12), 1351-64.
25. Vick, B.; Weber, A.; Urbanik, T.; Maass, T.; Teufel, A.; Krammer, P. H.; Opferman, J. T.; Schuchmann, M.; Galle, P. R.; Schulze-Bergkamen, H., Knockout of myeloid cell leukemia-1 induces liver damage and increases apoptosis susceptibility of murine hepatocytes. Hepatology 2009, 49 (2), 627-36.
26 clinicaltrials.gov/ct2/show/NCT03797261?cond=AMG176&draw=2&rank=1.
27 www.amgen.com/media/news-releases/2019/09/amgen-highlights-new-data-from-kyprolis-carfilzomib-and-oncology-pipeline-at-imw-2019/.
28. (a) Tron, A. E.; Belmonte, M. A.; Adam, A.; Aquila, B. M.; Boise, L. H.; Chiarparin, E.; Cidado, J.; Embrey, K. J.; Gangl, E.; Gibbons, F. D.; Gregory, G. P.; Hargreaves, D.; Hendricks, J. A.; Johannes, J. W.; Johnstone, R. W.; Kazmirski, S. L.; Kettle, J. G.; Lamb, M. L.; Matulis, S. M.; Nooka, A. K.; Packer, M. J.; Peng, B.; Rawlins, P. B.; Robbins, D. W.; Schuller, A. G.; Su, N.; Yang, W.; Ye, Q.; Zheng, X.; Secrist, J. P.; Clark, E. A.; Wilson, D. M.; Fawell, S. E.; Hird, A. W. Discovery of Mcl-1-specific inhibitor AZD5991 and preclinical activity in multiple myeloma and acute myeloid leukemia. Nat Commun. 2018, 9, 5341-5355.; (b) clinicaltrials.gov/ct2/show/NCT03218683?cond=AZD5991&draw=2&rank=1.
29. Blombery, P.; Birkinshaw, R. W.; Nguyen, T.; Gong, J. N.; Thompson, E. R.; Xu, Z.; Westerman, D. A.; Czabotar, P. E.; Dickinson, M.; Huang, D. C. S.; Seymour, J. F.; Roberts, A. W., Characterization of a novel venetoclax resistance mutation (BCL2 Phe104Ile) observed in follicular lymphoma. Br J Haematol 2019, 186 (6), e188-e191.
30 Blombery, P.; Anderson, M. A.; Gong, J. N.; Thijssen, R.; Birkinshaw, R. W.; Thompson, E. R.; Teh, C. E.; Nguyen, T.; Xu, Z.; Flensburg, C.; Lew, T. E.; Majewski, I. J.; Gray, D. H. D.; Westerman, D. A.; Tam, C. S.; Seymour, J. F.; Czabotar, P. E.; Huang, D. C. S.; Roberts, A. W., Acquisition of the Recurrent Gly 101Val Mutation in BCL2 Confers Resistance to Venetoclax in Patients with Progressive Chronic Lymphocytic Leukemia. Cancer Discov 2019, 9 (3), 342-353.
31. Wong, C. H.; Siah, K. W.; Lo, A. W., Estimation of clinical trial success rates and related parameters. Biostatistics 2019, 20 (2), 273-286.
32. Lin, A.; Giuliano, C. J.; Palladino, A.; John, K. M.; Abramowicz, C.; Yuan, M. L.; Sausville, E. L.; Lukow, D. A.; Liu, L.; Chait, A. R.; Galluzzo, Z. C.; Tucker, C.; Sheltzer, J. M., Off-target toxicity is a common mechanism of action of cancer drugs undergoing clinical trials. Sci Transl Med 2019, 11 (509).
33. Clavel, J., Progress in the epidemiological understanding of gene-environment interactions in major diseases: cancer. C R Biol 2007, 330 (4), 306-17.
34. Pollyea, D. A.; Amaya, M.; Strati, P.; Konopleva, M. Y., Venetoclax for AML: changing the treatment paradigm. Blood Adv 2019, 3 (24), 4326-4335.
35 Birkinshaw, R. W.; Gong, J. N.; Luo, C. S.; Lio, D.; White, C. A.; Anderson, M. A.; Blombery, P.; Lessene, G.; Majewski, I. J.; Thijssen, R.; Roberts, A. W.; Huang, D. C. S.; Colman, P. M.; Czabotar, P. E., Structures of BCL-2 in complex with venetoclax reveal the molecular basis of resistance mutations. Nat Commun 2019, 10 (1), 2385.
36 Anighoro, A.; Bajorath, J.; Rastelli, G., Polypharmacology: challenges and opportunities in drug discovery. J Med Chem 2014, 57 (19), 7874-87.
37 Proschak, E.; Stark, H.; Merk, D., Polypharmacology by Design: A Medicinal Chemist's Perspective on Multitargeting Compounds. J Med Chem 2019, 62 (2), 420-444.
38 Paiva, S. L.; Crews, C. M., Targeted protein degradation: elements of PROTAC design. Curr Opin Chem Biol 2019, 50, 111-119.
39 Zou, Y.; Ma, D.; Wang, Y., The PROTAC technology in drug development. Cell Biochem Funct 2019, 37 (1), 21-30.
40. Zhou, R.; Fang, S.; Zhang, M.; Zhang, Q.; Hu, J.; Wang, M.; Wang, C.; Zhu, J.; Shen, A.; Chen, X.; Zheng, C., Design, synthesis, and bioactivity evaluation of novel Bcl-2/HDAC dual-target inhibitors for the treatment of multiple myeloma. Bioorg Med Chem Lett 2019, 29 (3), 349-352.
41. Khan, S.; Zhang, X.; Lv, D.; Zhang, Q.; He, Y.; Zhang, P.; Liu, X.; Thummuri, D.; Yuan, Y.; Wiegand, J. S.; Pei, J.; Zhang, W.; Sharma, A.; McCurdy, C. R.; Kuruvilla, V. M.; Baran, N.; Ferrando, A. A.; Kim, Y. M.; Rogojina, A.; Houghton, P. J.; Huang, G.; Hromas, R.; Konopleva, M.; Zheng, G.; Zhou, D., A selective BCL-XL PROTAC degrader achieves safe and potent antitumor activity. Nat Med 2019, 25 (12), 1938-1947.
42. Yoshimoto, G.; Miyamoto, T.; Jabbarzadeh-Tabrizi, S.; Iino, T.; Rocnik, J. L.; Kikushige, Y.; Mori, Y.; Shima, T.; Iwasaki, H.; Takenaka, K.; Nagafuji, K.; Mizuno, S.; Niiro, H.; Gilliland, G. D.; Akashi, K., FLT3-ITD up-regulates MCL-1 to promote survival of stem cells in acute myeloid leukemia via FLT3-ITD-specific STAT5 activation. Blood 2009, 114 (24), 5034-43.
43. Sale, M. J.; Minihane, E.; Monks, N. R.; Gilley, R.; Richards, F. M.; Schifferli, K. P.; Andersen, C. L.; Davies, E. J.; Vicente, M. A.; Ozono, E.; Markovets, A.; Dry, J. R.; Drew, L.; Flemington, V.; Proia, T.; Jodrell, D. I.; Smith, P. D.; Cook, S. J., Targeting melanoma's MCL1 bias unleashes the apoptotic potential of BRAF and ERK1/2 pathway inhibitors. Nat Commun 2019, 10 (1), 5167.
44. Seipel, K.; Schmitter, K.; Bacher, U.; Pabst, T., Rationale for a Combination Therapy Consisting of MCL1- and MEK-Inhibitors in Acute Myeloid Leukemia. Cancers (Basel) 2019, 11 (11).
45. Bogenberger, J.; Whatcott, C.; Hansen, N.; Delman, D.; Shi, C. X.; Kim, W.; Haws, H.; Soh, K.; Lee, Y. S.; Peterson, P.; Siddiqui-Jain, A.; Weitman, S.; Stewart, K.; Bearss, D.; Mesa, R.; Warner, S.; Tibes, R., Combined venetoclax and alvocidib in acute myeloid leukemia. Oncotarget 2017, 8 (63), 107206-107222.
46 Dey, J.; Deckwerth, T. L.; Kerwin, W. S.; Casalini, J. R.; Merrell, A. J.; Grenley, M. O.; Burns, C.; Ditzler, S. H.; Dixon, C. P.; Beirne, E.; Gillespie, K. C.; Kleinman, E. F.; Klinghoffer, R. A., Voruciclib, a clinical stage oral CDK9 inhibitor, represses MCL-1 and sensitizes high-risk Diffuse Large B-cell Lymphoma to BCL2 inhibition. Sci Rep 2017, 7 (1), 18007.
47. Phillips, D. C.; Jin, S.; Gregory, G. P.; Zhang, Q.; Xue, J.; Zhao, X.; Chen, J.; Tong, Y.; Zhang, H.; Smith, M.; Tahir, S. K.; Clark, R. F.; Penning, T. D.; Devlin, J. R.; Shortt, J.; Hsi, E. D.; Albert, D. H.; Konopleva, M.; Johnstone, R. W.; Leverson, J. D.; Souers, A. J., A novel CDK9 inhibitor increases the efficacy of venetoclax (ABT-199) in multiple models of hematologic malignancies. Leukemia 2019.
48. Pan, R.; Ruvolo, V.; Mu, H.; Leverson, J. D.; Nichols, G.; Reed, J. C.; Konopleva, M.; Andreeff, M., Synthetic Lethality of Combined Bcl-2 Inhibition and p53 Activation in AML: Mechanisms and Superior Antileukemic Efficacy. Cancer Cell 2017, 32 (6), 748-760 e6.
49. Lehmann, C.; Friess, T.; Birzele, F.; Kiialainen, A.; Dangl, M., Superior anti-tumor activity of the MDM2 antagonist idasanutlin and the Bcl-2 inhibitor venetoclax in p53 wild-type acute myeloid leukemia models. J Hematol Oncol 2016, 9 (1), 50.
50 Schwartz, J.; Niu, X.; Walton, E.; Hurley, L.; Lin, H.; Edwards, H.; Taub, J. W.; Wang, Z.; Ge, Y., Synergistic anti-leukemic interactions between ABT-199 and panobinostat in acute myeloid leukemia ex vivo. Am J Transl Res 2016, 8 (9), 3893-3902.
51. (a) Kawase, T.; Nakazawa, T.; Eguchi, T.; Tsuzuki, H.; Ueno, Y.; Amano, Y.; Suzuki, T.; Mori, M.; Yoshida, T. Effect of Fms-like tyrosine kinase 3 (FLT3) ligand (FL) on antitumor activity of gilteritinib, a FLT3 inhibitor, in mice xenografted with FL-overexpressing cells. Oncotarget 2019, 10, 6111-6123; (b) Zhu, R.; Li, L.; Nguyen, B.; Duffield, A. S.; Small, D., Gilteritinib and venetoclax synergize to eliminate FLT3/ITD+leukemia cells through BIM. Blood 2019; 134 (Supplement_1): 2564.
52. Ohren, J. F.; Chen, H.; Pavlovsky, A.; Whitehead, C.; Zhang, E.; Kuffa, P.; Yan, C.; McConnell, P.; Spessard, C.; Banotai, C.; Mueller, W. T.; Delaney, A.; Omer, C.; Sebolt-Leopold, J.; Dudley, D. T.; Leung, I. K.; Flamme, C.; Warmus, J.; Kaufman, M.; Barrett, S.; Tecle, H.; Hasemann, C. A., Structures of human MAP kinase kinase 1 (MEK1) and MEK2 describe novel noncompetitive kinase inhibition. Nat Struct Mol Biol 2004, 11 (12), 1192-7; (b) https://clinicaltrials.gov/ct2/results?cond=&term=PD+0325901&cntry=&state=&city=&dist=
53. (a) Wyatt, P. G.; Woodhead, A. J.; Berdini, V.; Boulstridge, J. A.; Carr, M. G.; Cross, D. M.; Davis, D. J.; Devine, L. A.; Early, T. R.; Feltell, R. E.; Lewis, E. J.; Mcmenamin, R. L.; Navarro, E. F.; O'Brien, M. A.; O'Reilly, M.; Reule, M.; Saxty, G.; Seavers, L. C. A.; Smith, D.; Squires, M. S.; Trewartha, G.; Walker, M. T.; Woolford, A. J., Identification of N-(4-Piperidinyl)-4-(2,6-Dichlorobenzoylamino)-1H-Pyrazole-3-Carboxamide (AT7519), a novel cyclin dependent kinase inhibitor using fragment-based X-ray crystallography and structure based drug design. J Med Chem 2008, 51, 4986-4999; (b) Squires, M. S.; Feltell, R. E.; Wallis, N. G.; Lewis, E. J.; Smith, D. M.; Cross, D. M.; Lyons, J. F.; Thompson, N. T., Biological characterization of AT7519, a small-molecule inhibitor of cyclin-dependent kinases, in human tumor cell lines. Mol Cancer Ther. 2009, 8, 324-332. (c) https://clinicaltrials.gov/ct2/show/NCT01627054?term=AT7519&draw=2&rank=4
54. Hai, Y.; Christianson, D. W., Histone deacetylase 6 structure and molecular basis of catalysis and inhibition. Nat Chem Biol 2016, 12 (9), 741-7.
55. Bhatia, S.; Krieger, V.; Groll, M.; Osko, J. D.; Ressing, N.; Ahlert, H.; Borkhardt, A.; Kurz, T.; Christianson, D. W.; Hauer, J.; Hansen, F. K., Discovery of the First-in-Class Dual Histone Deacetylase-Proteasome Inhibitor. J Med Chem 2018, 61 (22), 10299-10309.
56. Pelz, N. F.; Bian, Z.; Zhao, B.; Shaw, S.; Tarr, J. C.; Belmar, J.; Gregg, C.; Camper, D. V.; Goodwin, C. M.; Arnold, A. L.; Sensintaffar, J. L.; Friberg, A.; Rossanese, O. W.; Lee, T.; Olejniczak, E. T.; Fesik, S. W., Discovery of 2-Indole-acylsulfonamide Myeloid Cell Leukemia 1 (Mcl-1) Inhibitors Using Fragment-Based Methods. J Med Chem 2016, 59 (5), 2054-66.
57 Lu, J.; Qian, Y.; Altieri, M.; Dong, H.; Wang, J.; Raina, K.; Hines, J.; Winkler, J. D.; Crew, A. P.; Coleman, K.; Crews, C. M., Hijacking the E3 Ubiquitin Ligase Cereblon to Efficiently Target BRD4. Chem Biol 2015, 22 (6), 755-63.
58. Hines, J.; Lartigue, S.; Dong, H.; Qian, Y.; Crews, C. M., MDM2-Recruiting PROTAC Offers Superior, Synergistic Antiproliferative Activity via Simultaneous Degradation of BRD4 and Stabilization of p53. Cancer Res 2019, 79 (1), 251-262.
59. Gadd, M. S.; Testa, A.; Lucas, X.; Chan, K. H.; Chen, W.; Lamont, D. J.; Zengerle, M.; Ciulli, A., Structural basis of PROTAC cooperative recognition for selective protein degradation. Nat Chem Biol 2017, 13 (5), 514-521.
60. Crews, C. M. B., Dennis; Ciulli, Alessio; Jorgensen, William; Gareiss, Peter C.; Van Molle, Inge; Gustafson, Jeffrey; Tae, Hyun-Seop; Michel, Julien; Hoyer, Dentin Wade; et al, Compounds and methods for the enhanced degradation of targeted proteins and other polypeptides by an E3 ubiquitin ligase, particularly targeting the von Hippel-Lindau E3 ubiquitin ligase using hydroxyproline analogs to disrupt the VHL/HIF-1α interaction. WO2013106643A2 2013.
61 clinicaltrials.gov/ct2/show/NCT03888612.
62. Fox, J. M.; Moynihan, J. R.; Mott, B. T.; Mazzone, J. R.; Anders, N. M.; Brown, P. A.; Rudek, M. A.; Liu, J. O.; Arav-Boger, R.; Posner, G. H.; Civin, C. I.; Chen, X., Artemisinin-derived dimer ART-838 potently inhibited human acute leukemias, persisted in vivo, and synergized with antileukemic drugs. Oncotarget 2016, 7 (6), 7268-79.
63. Mott, B. T.; He, R.; Chen, X.; Fox, J. M.; Civin, C. I.; Arav-Boger, R.; Posner, G. H., Artemisinin-derived dimer phosphate esters as potent anti-cytomegalovirus (anti-CMV) and anti-cancer agents: a structure-activity study. Bioorg Med Chem 2013, 21 (13), 3702-7.
64 Sharon, D.; Cathelin, S.; Mirali, S.; Di Trani, J. M.; Yanofsky, D. J.; Keon, K. A.; Rubinstein, J. L.; Schimmer, A. D.; Ketela, T.; Chan, S. M., Inhibition of mitochondrial translation overcomes venetoclax resistance in AML through activation of the integrated stress response. Sci Transl Med 2019, 11 (516).
65 Seipel, K.; Marques, M. A. T.; Sidler, C.; Mueller, B. U.; Pabst, T., MDM2- and FLT3-inhibitors in the treatment of FLT3-ITD acute myeloid leukemia, specificity and efficacy of NVP-HDM201 and midostaurin. Haematologica 2018, 103 (11), 1862-1872.
66. Pan, R.; Hogdal, L. J.; Benito, J. M.; Bucci, D.; Han, L.; Borthakur, G.; Cortes, J.; DeAngelo, D. J.; Debose, L.; Mu, H.; Dohner, H.; Gaidzik, V. I.; Galinsky, I.; Golfman, L. S.; Haferlach, T.; Harutyunyan, K. G.; Hu, J.; Leverson, J. D.; Marcucci, G.; Muschen, M.; Newman, R.; Park, E.; Ruvolo, P. P.; Ruvolo, V.; Ryan, J.; Schindela, S.; Zweidler-Mckay, P.; Stone, R. M.; Kantarjian, H.; Andreeff, M.; Konopleva, M.; Letai, A. G., Selective BCL-2 inhibition by ABT-199 causes on-target cell death in acute myeloid leukemia. Cancer Discov 2014, 4 (3), 362-75.
67. Anstee, N. S.; Bilardi, R. A.; Ng, A. P.; Xu, Z.; Robati, M.; Vandenberg, C. J.; Cory, S., Impact of elevated anti-apoptotic MCL-1 and BCL-2 on the development and treatment of MLL-AF9 AML in mice. Cell Death Differ 2019, 26 (7), 1316-1331.
68. Seipel, K.; Marques, M. A. T.; Sidler, C.; Mueller, B. U.; Pabst, T., The Cellular p53 Inhibitor MDM2 and the Growth Factor Receptor FLT3 as Biomarkers for Treatment Responses to the MDM2-Inhibitor Idasanutlin and the MEK1 Inhibitor Cobimetinib in Acute Myeloid Leukemia. Cancers (Basel) 2018, 10 (6).
69. Gopalakrishnapillai, A.; Kolb, E. A.; McCahan, S. M.; Barwe, S. P., Epigenetic drug combination induces remission in mouse xenograft models of pediatric acute myeloid leukemia. Leuk Res 2017, 58, 91-97.
70. Georgantas, R. W., 3rd; Hildreth, R.; Morisot, S.; Alder, J.; Liu, C. G.; Heimfeld, S.; Calin, G. A.; Croce, C. M.; Civin, C. I., CD34+ hematopoietic stem-progenitor cell microRNA expression and function: a circuit diagram of differentiation control. Proc Natl Acad Sci USA 2007, 104 (8), 2750-5.

71 Kim, M.; Tan, Y. S.; Cheng, W. C.; Kingsbury, T. J.; Heimfeld, S.; Civin, C. I., MIR144 and MIR451 regulate human erythropoiesis via RAB14. Br J Haematol 2015, 168 (4), 583-97.
72 Cao, X.; Yap, J. L.; Newell-Rogers, M. K.; Peddaboina, C.; Jiang, W.; Papaconstantinou, H. T.; Jupitor, D.; Rai, A.; Jung, K. Y.; Tubin, R. P.; Yu, W.; Vanommeslaeghe, K.; Wilder, P. T.; Mackerell, A. D., Jr.; Fletcher, S.; Smythe, R. W., The novel BH3 alpha-helix mimetic JY-1-106 induces apoptosis in a subset of cancer cells (lung cancer, colon cancer and mesothelioma) by disrupting Bcl-xL and Mcl-1 protein-protein interactions with Bak. Mol Cancer 2013, 12 (1), 42.
73. Chen, L.; Wilder, P. T.; Drennen, B.; Tran, J.; Roth, B. M.; Chesko, K.; Shapiro, P.; Fletcher, S., Structure-based design of 3-carboxy-substituted 1,2,3,4-tetrahydroquinolines as inhibitors of myeloid cell leukemia-1 (Mcl-1). Org Biomol Chem 2016, 14 (24), 5505-10.
74. Conlon, I. L.; Van Eker, D.; Abdelmalak, S.; Murphy, W. A.; Bashir, H.; Sun, M.; Chauhan, J.; Varney, K. M.; Godoy-Ruiz, R.; Wilder, P. T.; Fletcher, S., Krohnke pyridines: Rapid and facile access to Mcl-1 inhibitors. Bioorg Med Chem Lett 2018, 28 (10), 1949-1953.
75. Drennen, B.; Scheenstra, J. A.; Yap, J. L.; Chen, L.; Lanning, M. E.; Roth, B. M.; Wilder, P. T.; Fletcher, S., Structural Re-engineering of the alpha-Helix Mimetic JY-1-106 into Small Molecules: Disruption of the Mcl-1-Bak-BH3 Protein-Protein Interaction with 2,6-Di-Substituted Nicotinates. ChemMedChem 2016, 11 (8), 827-33.
76 Lanning, M. E.; Yu, W.; Yap, J. L.; Chauhan, J.; Chen, L.; Whiting, E.; Pidugu, L. S.; Atkinson, T.; Bailey, H.; Li, W.; Roth, B. M.; Hynicka, L.; Chesko, K.; Toth, E. A.; Shapiro, P.; Mackerell, A. D., Jr.; Wilder, P. T.; Fletcher, S., Structure-based design of N-substituted 1-hydroxy-4-sulfamoyl-2-naphthoates as selective inhibitors of the Mcl-1 oncoprotein. Eur J Med Chem 2016, 113, 273-92.
77. Whiting, E.; Raje, M. R.; Chauhan, J.; Wilder, P. T.; Van Eker, D.; Hughes, S. J.; Bowen, N. G.; Vickers, G. E. A.; Fenimore, I. C.; Fletcher, S., Discovery of Mcl-1 inhibitors based on a thiazolidine-2,4-dione scaffold. Bioorg Med Chem Lett 2018, 28 (3), 523-528.
78 Conlon, I. L.; Drennen, B.; Lanning, M. E.; Hughes, S.; Rothhaas, R.; Wilder, P. T.; MacKerell, A. D. Jr; Fletcher, S. Rationally designed polypharmacology: α-Helix mimetics as dual inhibitors of the oncoproteins Mcl-1 and HDM2. ChemMedChem 2020, 15, 1691-1698.
79. Winzker, M.; Friese, A.; Koch, U.; Janning, P.; Ziegler, S.; Waldmann, H. Development of a PDEd-targeting PROTACs that impair lipid metabolism. Angew. Chem. Int. Ed. 2020, 59, 5595-5601.
80. Schoenwaelder, S. M.; Jarman, K. E.; Gardiner, E. E.; Hua, M.; Qiao, J.; White, M. J.; Josefsson, E. C.; Alwis, I.; Ono, A.; Willcox, A.; Andrews, R. K.; Mason, K. D.; Salem, H. H.; Huang, D. C.; Kile, B. T.; Roberts, A. W.; Jackson, S. P., Bcl-xL-inhibitory BH3 mimetics can induce a transient thrombocytopathy that undermines the hemostatic function of platelets. Blood 2011, 118 (6), 1663-74.
81 Nikolovska-Coleska, Z.; Wang, R.; Fang, X.; Pan, H.; Tomita, Y.; Li, P.; Roller, P. P.; Krajewski, K.; Saito, N. G.; Stuckey, J. A. Anal. Biochem. 2004, 332, 261-273

It is claimed:

1. A compound of formula (I), or comprising a substructure of formula (I), or a pharmaceutically acceptable salt, solvate, hydrate, cocrystal, or prodrug thereof: 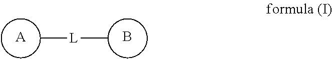
wherein in formula (I):
A comprises a venetoclax moiety;
L is a linking group; and
B comprises a Mcl-1 protein inhibitor moiety, Mcl-1 protein downregulator moiety, or protein degradation moiety, wherein the venetoclax moiety thereof, and the Mcl-1 protein inhibitor moiety, Mcl-1 protein downregulator moiety, or protein degradation moiety thereof are each connected to L at any chemically feasible site.

2. The compound of claim 1, wherein the compound of formula (I) is a compound of formula (10), or a pharmaceutically acceptable salt, solvate, hydrate, cocrystal, or prodrug thereof: 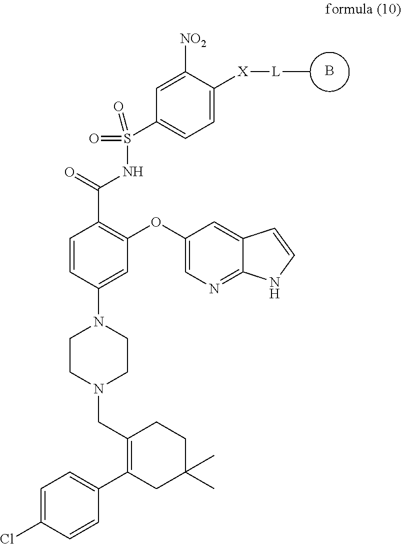
wherein in formula (10):
X is —NH— or 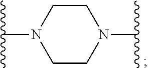 [SMILES: *N1CCN(*)CC1]
and
L is a linking group; and
B comprises a Mcl-1 protein inhibitor moiety, Mcl-1 protein downregulator moiety, or protein degradation moiety, wherein the venetoclax moiety thereof, and the Mcl-1 protein inhibitor moiety, Mcl-1 protein downregulator moiety, or protein degradation moiety thereof are each connected to L at any chemically feasible site.

3. The compound of claim 1 or 2, wherein the compound of formula (I) is a compound of formula (100) or formula (110), or a pharmaceutically acceptable salt, solvate, hydrate, cocrystal, or prodrug thereof: 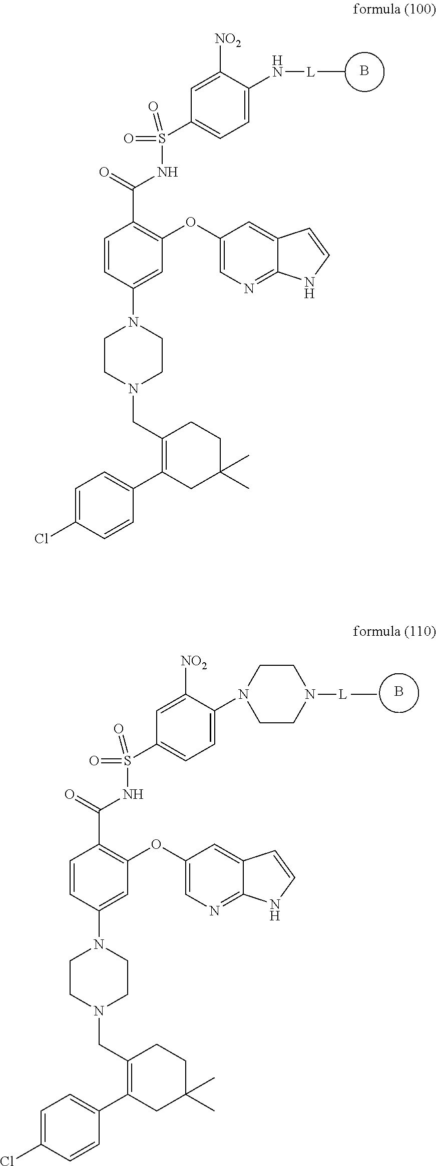
wherein in formula (100) and formula (110):
L is a linking group; and
B comprises a Mcl-1 protein inhibitor moiety, Mcl-1 protein downregulator moiety, or protein degradation moiety, wherein the venetoclax moiety thereof, and the Mcl-1 protein inhibitor moiety, Mcl-1 protein downregulator moiety, or protein degradation moiety thereof are each connected to L at any chemically feasible site.

4. The compound of claims 1-3, wherein L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, -cycloalkyl-, -heterocyclyl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR^{b})O—, (O)P(O—)₃, —O(S)P(OR^{b})O—, and (S)P(O—)₃, wherein w is 1 or 2, and R^{b} is independently hydrogen, optionally substituted alkyl, or optionally substituted aryl.

5. The compound of any one of claims 1-4, wherein L comprises one or more linking groups selected from —C₁₋₁₀ alkyl-, —O—C₁₋₁₀ alkyl-, —O—, —C(O)—, —N(R^{b})C(O)— wherein R^{b} is hydrogen or optionally substituted alkyl, —C(O)N(R^{b})— wherein R^{b} is hydrogen or optionally substituted alkyl, and —N(R^{b})— wherein R^{b} is hydrogen or optionally substituted alkyl.

6. The compound of any one of claims 1-4, wherein L comprises one or more linking groups selected from 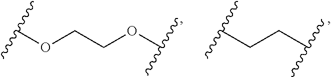
—NHC(O)—, and —C(O)NH—.

7. The compound of any one of claims 1-4, wherein L comprises one or more linking groups selected from —NHC(O)—, —C(O)NH—, 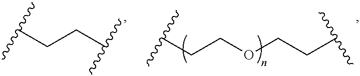
wherein n=1-5.

8. The compound of any one of claims 1-4, wherein L comprises one or more linking groups selected from —NHC(O)—, —C(O)NH—, 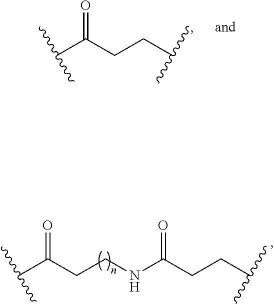
wherein n=0-6.

9. The compound of claims 7-8, wherein B comprises a Mcl-1 protein inhibitor moiety selected from AZD5991, AMG176, and MIK665, and any substructure thereof.

10. The compound of claims 7-8, wherein B comprises a Mcl-1 protein downregulator moiety selected from a CDK9 inhibitor, a MEK 1/2 inhibitor, a HDAC inhibitor, a FLT3 inhibitor, MDM2 inhibitor, JAK1/2 inhibitor, STAT3 inhibitor, ERK1/2 inhibitor, dual PI3K/mTOR inhibitor, and any substructure thereof.

11. The compound of claim 10, wherein the CDK9 inhibitor is selected from alvocidib (flavopiridol), SNS-032, AT7519, TG02, PHA 767491, PHA-793887, PHA-848125, BAY 1143572, BAY 1112054, Cdk9 inhibitor II (CAS 140651-18-9 from Calbiochem), DRB, AZD-5438, dinaciclib, LY2857785, purvalanol B, CDKI-71, CDKI-73, CAN508, FIT-039, CYC065, P276-00, 3,4-dimethyl-5-[2-(4-piperazin-1-yl-phenylamino)-pyrimidin-4-yl]-3H-thiazol-2-one, wogonin, apigenin, chrysin, luteolin, 4-methyl-5-[2-(3-nitroanilino)pyrimidin-4-yl]-1,3-thiazol-2-amine, shRNAs against CDK9, anti-sense mRNA against CDK9, anti-CDK9 antibodies, and substructure thereof.

12. The compound of claim 10, wherein the MEK 1/2 inhibitor is selected from cobimetinib (GDC-0973), PD334581, CI-1040, AZD6244, PD318088, PD98059, RDEA119, 6-Methoxy-7-(3-morpholin-4-yl-propoxy)-4-(4-phenoxy-phenylamino)-quinoline-3-carbonitrile, and 4-[3-Chloro-4-(1-methyl-1H-imidazol-2-ylsulfanyl)-phenylamino]-6-methoxy-7-(3-morpholin-4-yl-propoxy)-quinoline-3-carbonitrile, and any substructure thereof.

13. The compound of claim 10, wherein the FLT3 inhibitor is selected from gilteritinib, AT7519, quizartinib, midostaurin, crenolanib, FLX-925 also known as AMG-925, G-749, and any substructure thereof.

14. The compound of claim 10, wherein the MDM2 inhibitor is selected from RG7112 (idasanutlin), PD334581, Nutlin-3a; RG7388; RO5503781; DS-3032b; MI-63; MI-126; MI-122; MI-142; MI-147; MI-18; MI-219; MI-220; MI-221; MI-773; and 3-(4-chlorophenyl)-3-((1-(hydroxymethyl)cyclopropyl)methoxy)-2-(4-nitrobenzyl)isoindolin-1-one; Serdemetan; AM-8553; CGM097, and any substructure thereof.

15. The compound of claim 10, wherein the HDAC inhibitor is selected from panobinostat, vorinostat, and rocilinostat (ACY-1215), and any substructure thereof.

16. The compound of claim 10, wherein the JAK1/2 inhibitor is selected from Ruxolitinib, Baricitinib, Tofacitinib, and any substructure thereof.

17. The compound of claim 10, wherein the STAT3 inhibitor is selected from WP1066, S3I-201, C1-C10, and any substructure thereof.

18. The compound of claim 10, wherein the ERK1/2 inhibitor is selected from WP1066AEZS-131, AEZS-136, BVD-523, SCH-722984, SCH-772984, SCH-900353 (MK-8353), and any substructure thereof.

19. The compound of claim 10, wherein the dual PI3K/mTOR inhibitor is selected from gedatolisib (PF-05212384; PKI-587), XL765, GDC-0980, BEZ235 (NVP-BEZ235), BGT226, GSK2126458, PF-04691502, and any substructure thereof.

20. The compound of any one of claims 1-19, wherein B comprises a moiety selected from AZD5991, SNS-032, AT7519, PD334581, gilteritinib, quizartinib, RG7112 (idasanutlin), panobinostat, AMG176, alvocidib (flavopiridol), cobimetinib (GDC-0973), and any substructure thereof.

21. The compound of claim 19, wherein B comprises a moiety selected from AZD5991, SNS-032, AT7519, PD334581, gilteritinib, quizartinib, RG7112 (idasanutlin), panobinostat, and any substructure thereof.

22. The compound of any one of claims 1-21, wherein B comprises a moiety selected from:

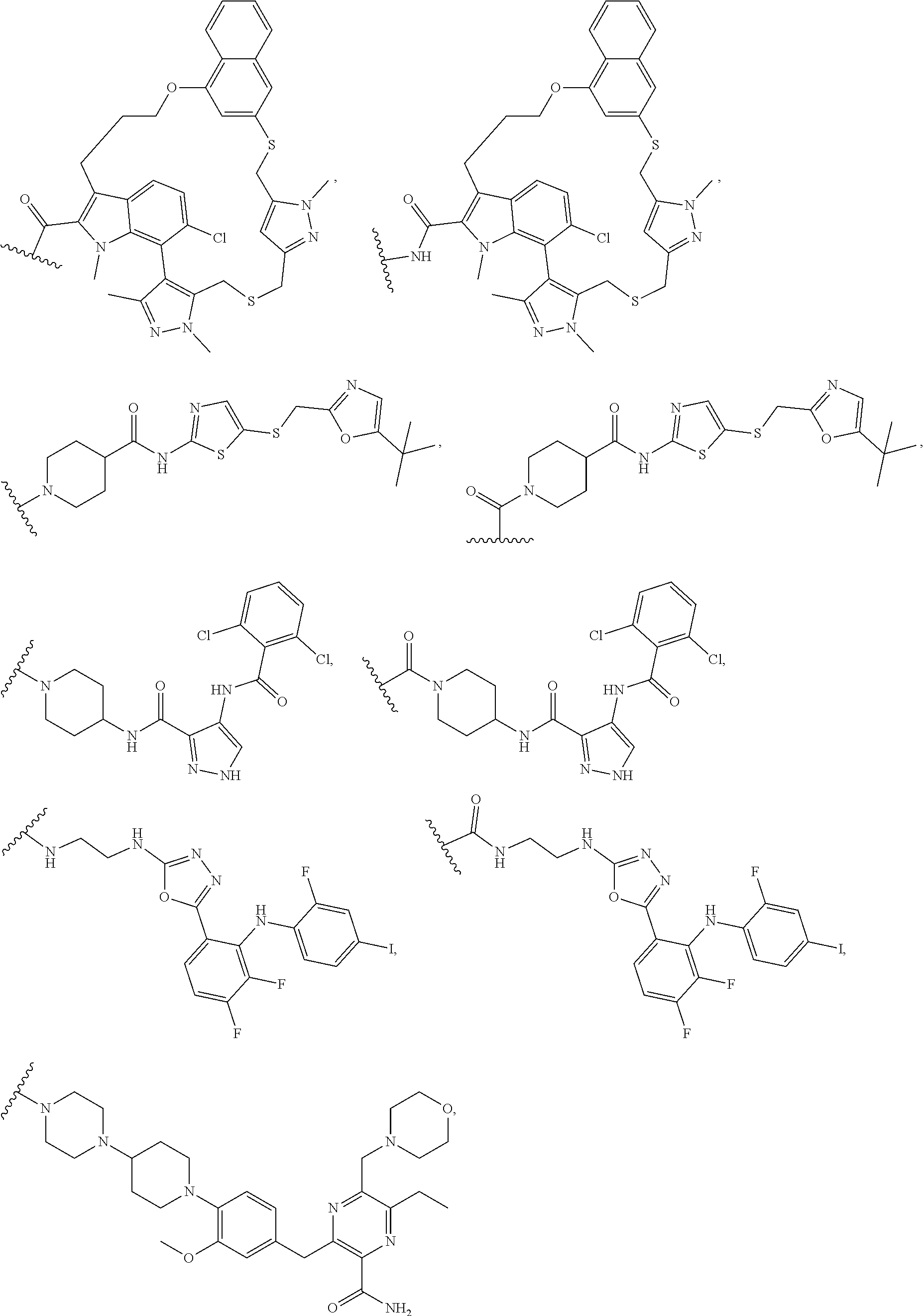

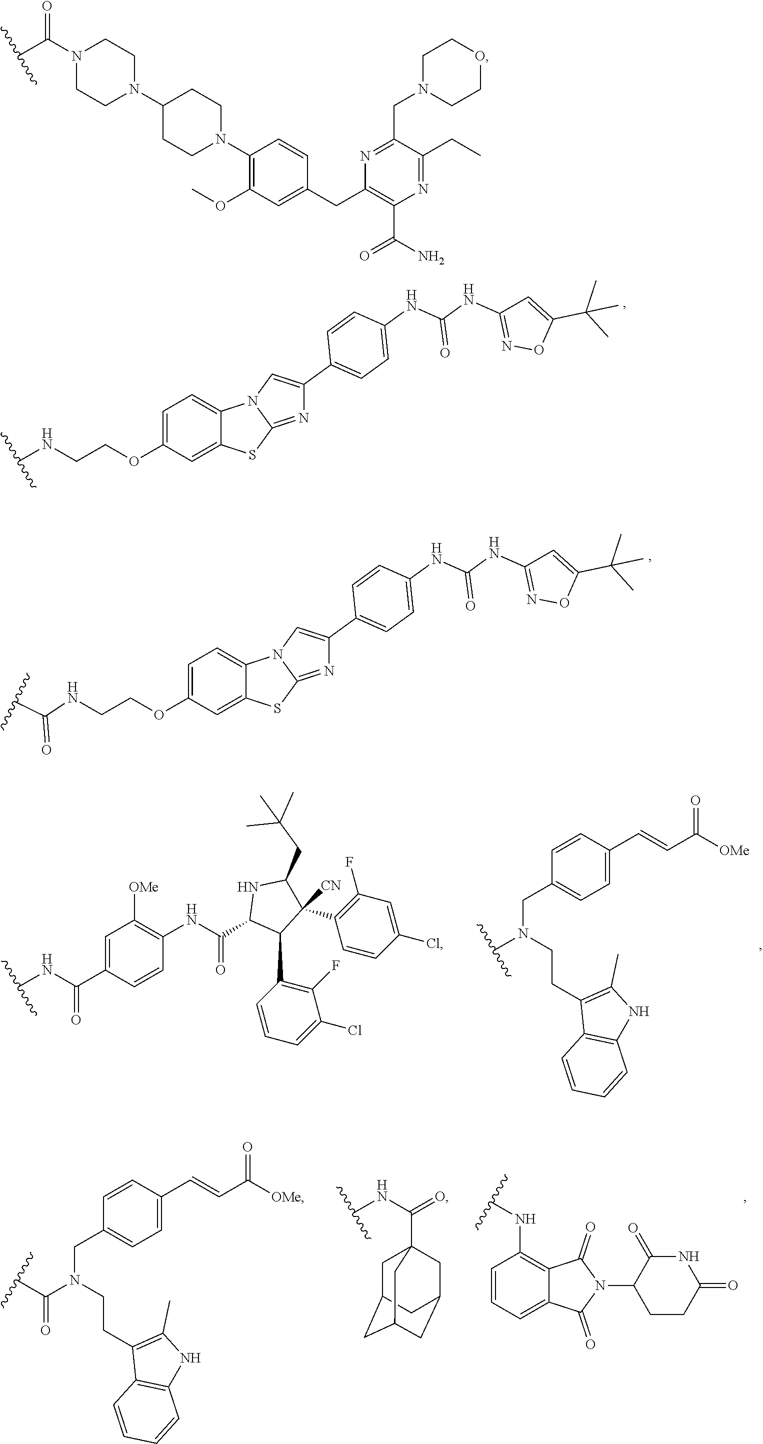

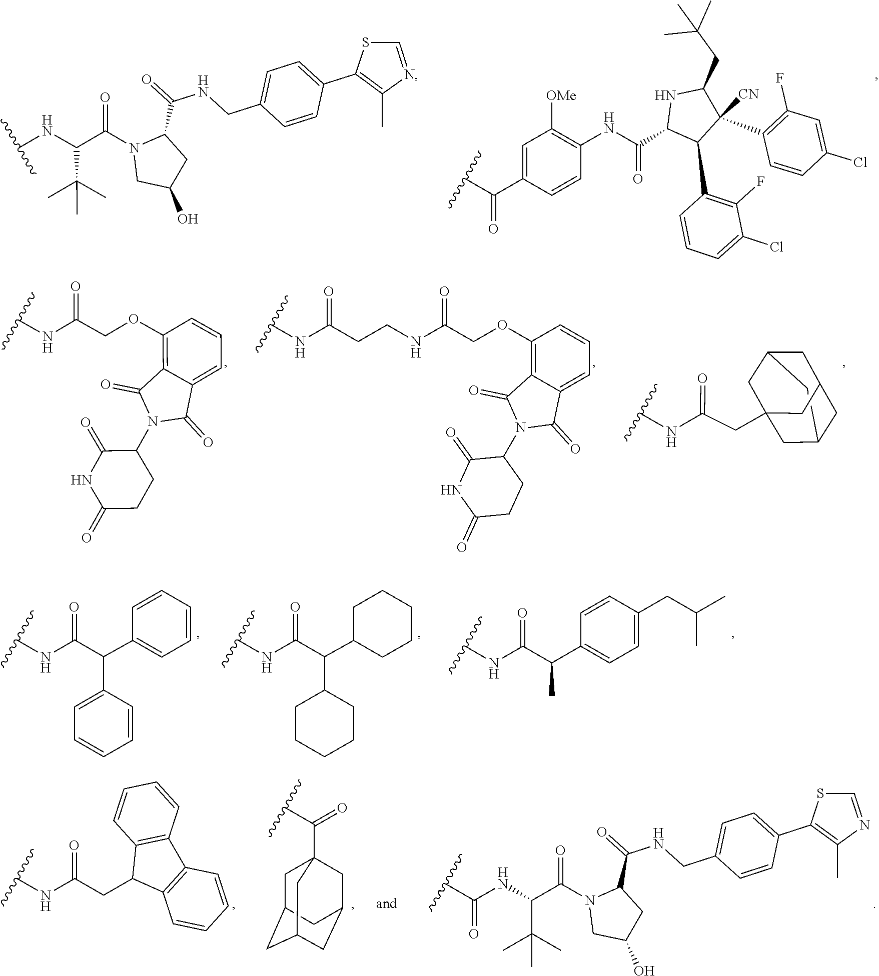

23. The compound of any one of claims 1-8, wherein the protein degradation moiety comprises a hydrophobic tagging group or moiety or a E3 ubiquitin ligase ligand moiety.

24. The compound of claim 23, wherein the hydrophobic tagging group or moiety comprises an adamantane moiety comprising

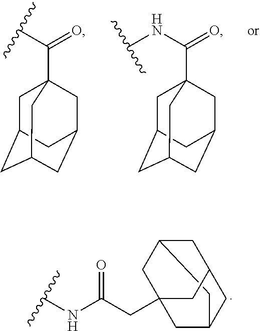 [SMILES: *C(=O)C12CC3CC(CC(C3)C1)C2 ; *NC(=O)C12CC3CC(CC(C3)C1)C2 ; *NC(=O)CC12CC3CC(CC(C3)C1)C2]

25. The compound of claim 23, wherein the E3 ubiquitin ligase ligand moiety is selected from a cereblon (CRBN) ligand, a mouse double minute 2 (MDM2) ligand, a Von Hippel-Lindau (VHL) ligand, and any substructure thereof.

26. The compound of claim 25, wherein the CRBN ligand is selected from thalidomide, lenalidomide, pomalidomide, and any substructure thereof.

27. The compound of claim 25, wherein the MDM2 ligand is selected from idasanutlin, RG7112, RG7388, MI 773/SAR 405838, AMG 232, DS-3032b, RO6839921, RO5045337, RO5503781, CGM-097, MK-8242, and any substructure thereof.

28. The compound of claim 25, wherein the VHL ligand is selected from VHL ligand 1 (VHL-1), VHL ligand 2 (VHL-2), VH032, and any substructure thereof.

29. The compound of any one of claims 1-8, wherein the E3 ubiquitin ligase ligand moiety is selected from thalidomide, idasanutlin, VHL ligand 1 (VHL-1), and any substructure thereof.

30. The compound of claim 29, wherein the E3 ubiquitin ligase ligand moiety comprises 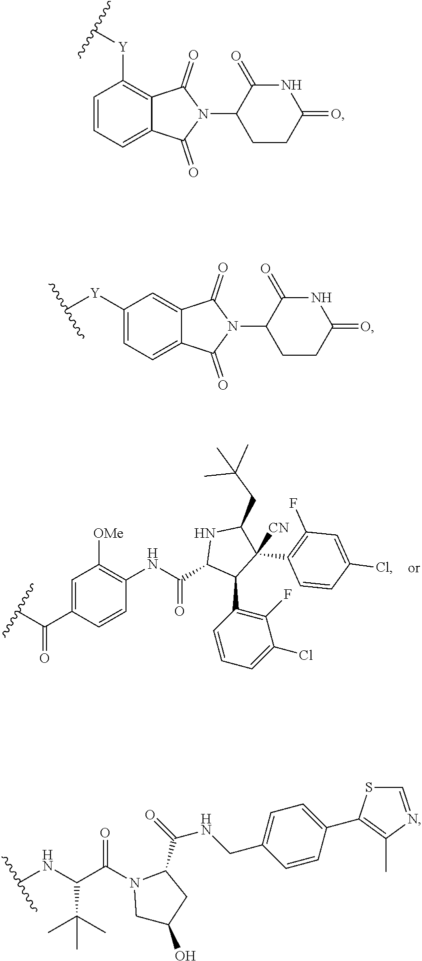
wherein Y is selected from —N(R)—, —N(H)—, and —O—, wherein R is optionally substituted alkyl.

31. The compound of claim 29, wherein the E3 ubiquitin ligase ligand moiety is selected from

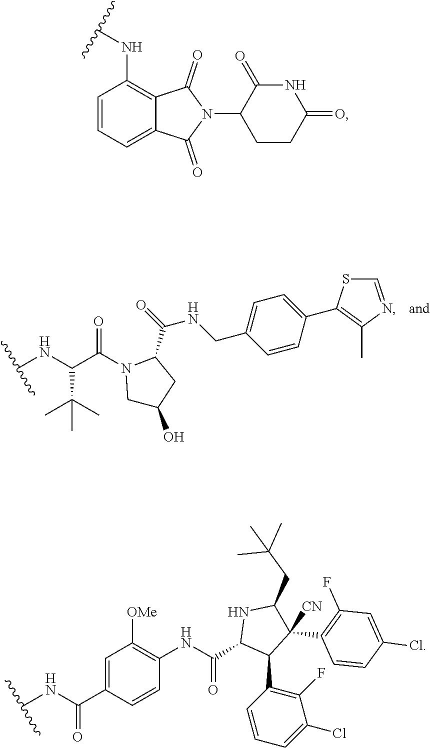

32. The compound of any one of claims 1-31, wherein the compound has a molecular weight not greater than about 2000 g/mol, or about 1900 g/mol, or about 1800 g/mol, or about 1700 g/mol, or about 1600 g/mol, or about 1500 g/mol, or about 1400 g/mol, or about 1300 g/mol, or about 1200 g/mol or about 1100 g/mol, or about 1000 g/mol.

33. The compound of claim 3, wherein the compound of formula (100) is a compound of any one of formula 1001-1162, or a pharmaceutically acceptable salt, solvate, hydrate, cocrystal, or prodrug thereof: 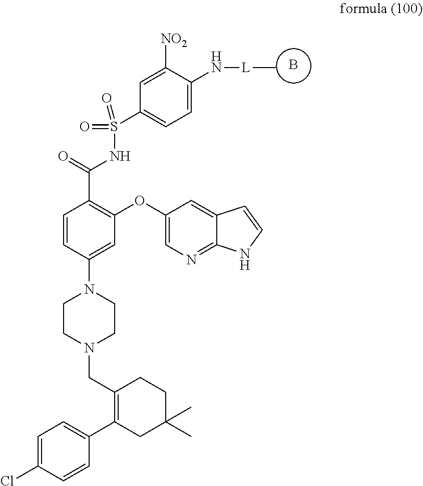
B is

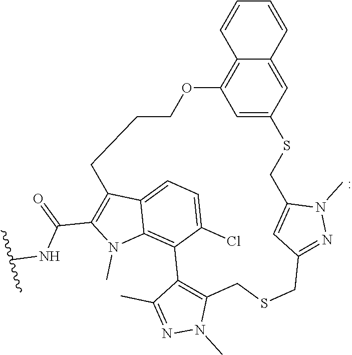 [SMILES: *NC(=O)c1c2c3ccc(Cl)c(c3n1C)-c1c(C)nn(C)c1CSCc1cc(n(C)n1)CSc1cc(c3ccccc3c1)OCCC2]

| Formula | | |
|---|---|---|
| No. | L | n |
| 1001 | 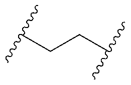 [SMILES: *CC*] | — |
| 1002 | 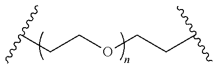 | 1 |
| 1003 | 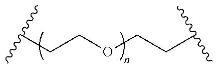 | 2 |
| 1004 | 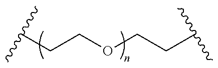 | 3 |
| 1005 | 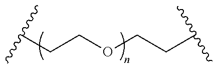 | 4 |
| 1006 | 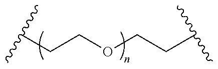 | 5 |

B is

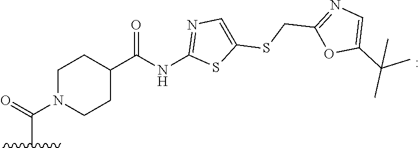 [SMILES: *C(=O)N1CCC(C(=O)Nc2ncc(SCc3ncc(C(C)(C)C)o3)s2)CC1]

| Formula | | |
|---|---|---|
| No. | L | n |
| 1007 | 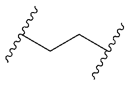 [SMILES: *CC*] | — |
| 1008 | 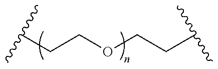 | 1 |
| 1009 | 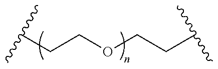 | 2 |
| 1010 | 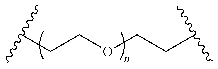 | 3 |
| 1011 | 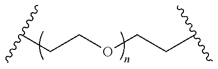 | 4 |
| 1012 | 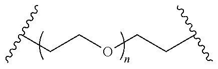 | 5 |

B is

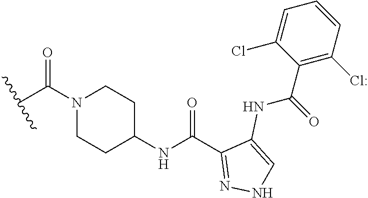

| Formula | | |
|---|---|---|
| No. | L | n |
| 1013 | 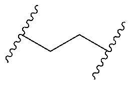 [SMILES: *CC*] | — |
| 1014 | 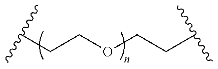 | 1 |
| 1015 | 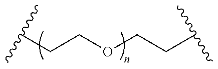 | 2 |
| 1016 | 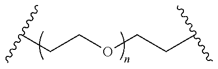 | 3 |
| 1017 | 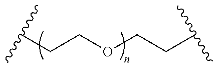 | 4 |
| 1018 | 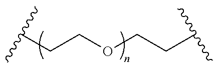 | 5 |

B is

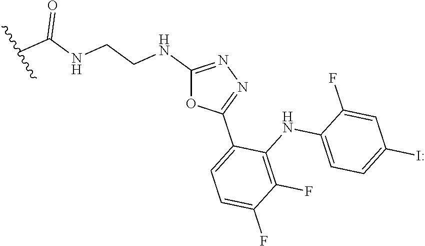

| Formula | | |
|---|---|---|
| No. | L | n |
| 1019 | 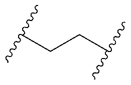 [SMILES: *CC*] | — |
| 1020 | 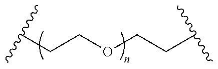 | 1 |
| 1021 | 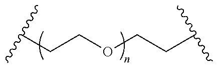 | 2 |
| 1022 | 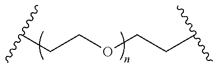 | 3 |
| 1023 | 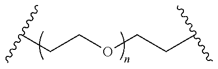 | 4 |
| 1024 | 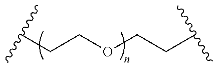 | 5 |

B is

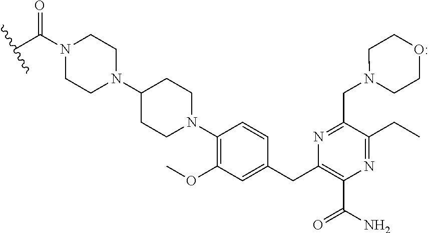

| Formula | | |
|---|---|---|
| No. | L | n |
| 1025 | 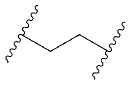 [SMILES: *CC*] | — |
| 1026 | 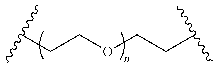 | 1 |
| 1027 | 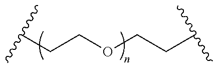 | 2 |
| 1028 | 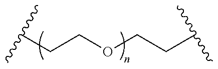 | 3 |
| 1029 | 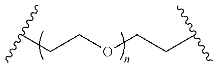 | 4 |
| 1030 | 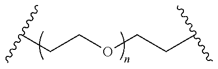 | 5 |

B is

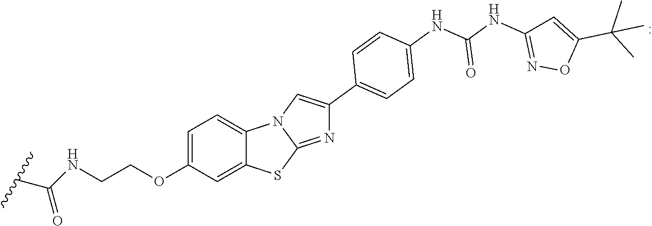 [SMILES: *C(=O)NCCOc1ccc2c(c1)sc1nc(-c3ccc(NC(=O)Nc4cc(C(C)(C)C)on4)cc3)cn12]

| Formula | | |
|---|---|---|
| No. | L | n |
| 1031 | 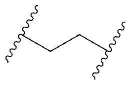 [SMILES: *CC*] | — |
| 1032 | 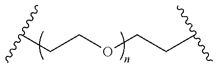 | 1 |
| 1033 | 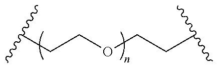 | 2 |
| 1034 | 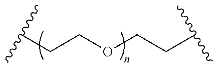 | 3 |
| 1035 | 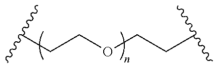 | 4 |
| 1036 | 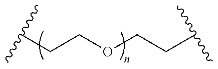 | 5 |

B is

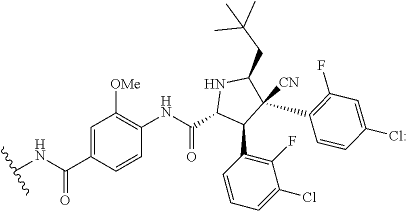

| Formula | | |
|---|---|---|
| No. | L | n |
| 1037 | 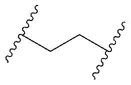 [SMILES: *CC*] | — |
| 1038 | 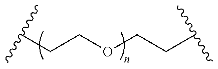 | 1 |
| 1039 | 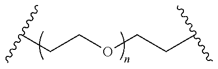 | 2 |
| 1040 | 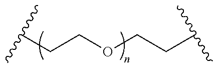 | 3 |
| 1041 | 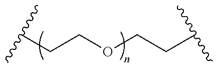 | 4 |
| 1042 | 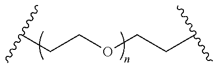 | 5 |

B is

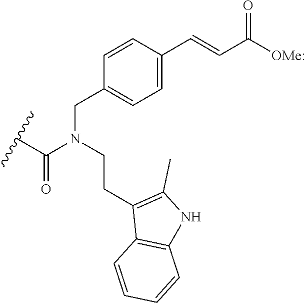

| Formula | | |
|---|---|---|
| No. | L | n |
| 1043 | 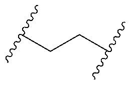 [SMILES: *CC*] | — |
| 1044 | 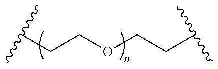 | 1 |
| 1045 | 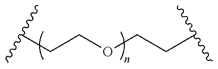 | 2 |
| 1046 | 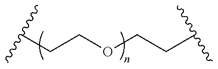 | 3 |
| 1047 | 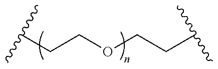 | 4 |
| 1048 | 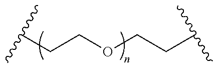 | 5 |

B is

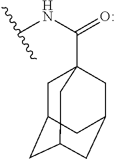

| Formula | | |
|---|---|---|
| No. | L | n |
| 1049 | 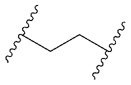 [SMILES: *CC*] | — |
| 1050 | 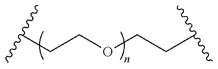 | 1 |
| 1051 | 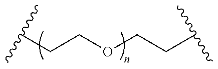 | 2 |
| 1052 | 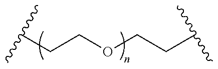 | 3 |
| 1053 | 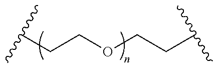 | 4 |
| 1054 | 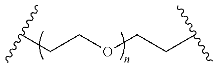 | 5 |

B is

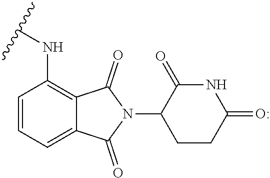

| Formula | | |
|---|---|---|
| No. | L | n |
| 1055 | 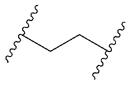 [SMILES: *CC*] | — |
| 1056 | 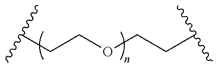 | 1 |
| 1057 | 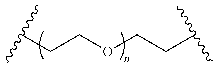 | 2 |
| 1058 | 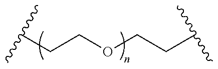 | 3 |
| 1059 | 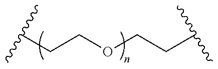 | 4 |
| 1060 | 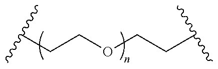 | 5 |

B is

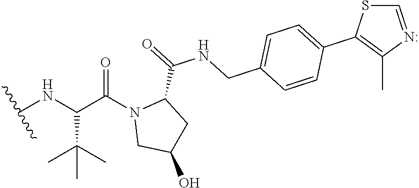

| Formula | | |
|---|---|---|
| No. | L | n |
| 1061 | 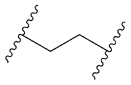 [SMILES: *CC*] | — |
| 1062 | 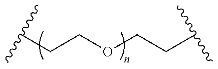 | 1 |
| 1063 | 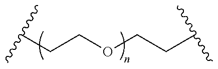 | 2 |
| 1064 | 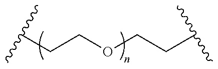 | 3 |
| 1065 | 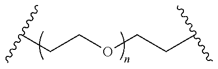 | 4 |
| 1066 | 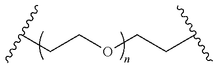 | 5 |

B is

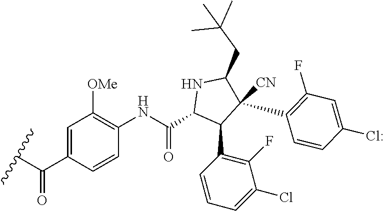

| Formula | | |
|---|---|---|
| No. | L | n |
| 1067 | 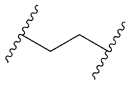 [SMILES: *CC*] | — |
| 1068 | 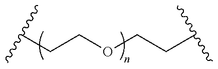 | 1 |
| 1069 | 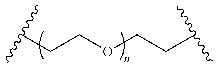 | 2 |
| 1070 | 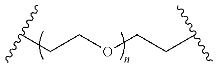 | 3 |
| 1071 | 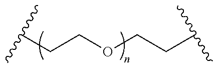 | 4 |
| 1072 | 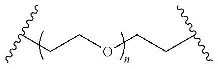 | 5 |

B is

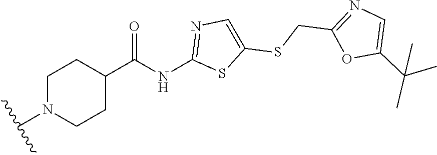 [SMILES: *N1CCC(C(=O)Nc2ncc(SCc3ncc(C(C)(C)C)o3)s2)CC1]

| Formula | | |
|---|---|---|
| No. | L | n |
| 1073 | 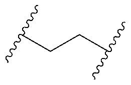 [SMILES: *CC*] | — |
| 1074 | 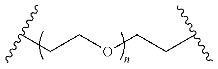 | 1 |
| 1075 | 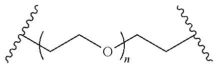 | 2 |
| 1076 | 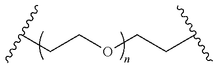 | 3 |
| 1077 | 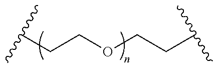 | 4 |
| 1078 | 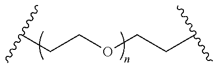 | 5 |

B is

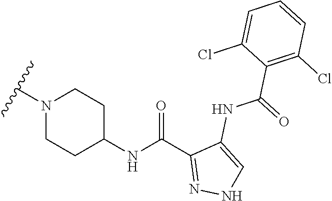 [SMILES: *N1CCC(NC(=O)c2n[nH]cc2NC(=O)c2c(Cl)cccc2Cl)CC1]

| Formula | | |
|---|---|---|
| No. | L | n |
| 1079 | 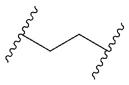 [SMILES: *CC*] | — |
| 1080 | 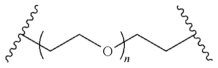 | 1 |
| 1081 | 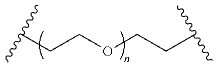 | 2 |
| 1082 | 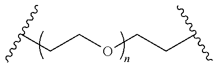 | 3 |
| 1083 | 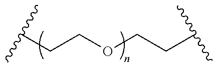 | 4 |
| 1084 | 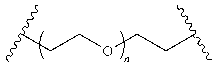 | 5 |

B is

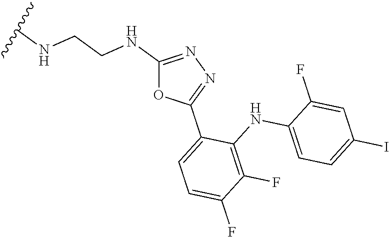 [SMILES: *NCCNc1nnc(-c2ccc(F)c(F)c2Nc2ccc(I)cc2F)o1]

| Formula | | |
|---|---|---|
| No. | L | n |
| 1085 | 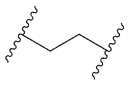 [SMILES: *CC*] | — |
| 1086 | 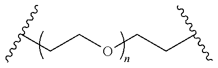 | 1 |
| 1087 | 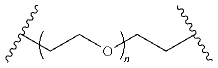 | 2 |
| 1088 | 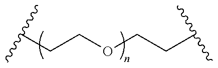 | 3 |
| 1089 | 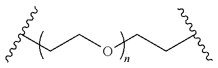 | 4 |
| 1090 | 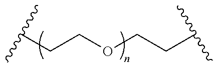 | 5 |

B is

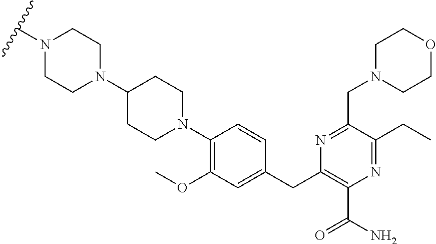 [SMILES: *N1CCN(C2CCN(c3ccc(Cc4nc(CN5CCOCC5)c(CC)nc4C(N)=O)cc3OC)CC2)CC1]

| Formula | | |
|---|---|---|
| No. | L | n |
| 1091 | 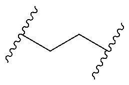 [SMILES: *CC*] | — |
| 1092 | 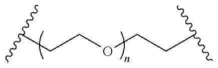 | 1 |
| 1093 | 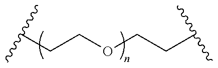 | 2 |
| 1094 | 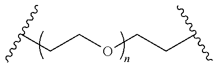 | 3 |
| 1095 | 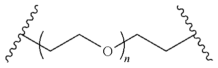 | 4 |
| 1096 | 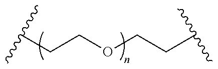 | 5 |

B is

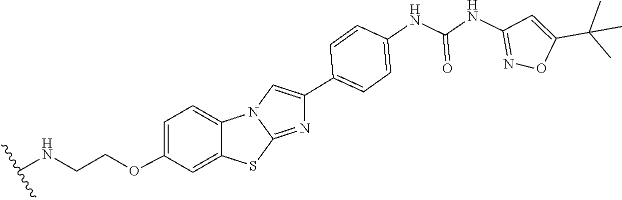 [SMILES: *NCCOc1ccc2c(c1)sc1nc(-c3ccc(NC(=O)Nc4cc(C(C)(C)C)on4)cc3)cn12]

| Formula | | |
|---|---|---|
| No. | L | n |
| 1097 | 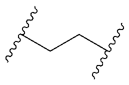 [SMILES: *CC*] | — |
| 1098 | 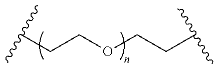 | 1 |
| 1099 | 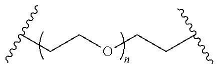 | 2 |
| 1100 | 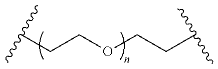 | 3 |
| 1101 | 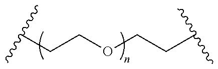 | 4 |
| 1102 | 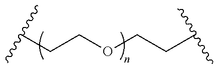 | 5 |

B is

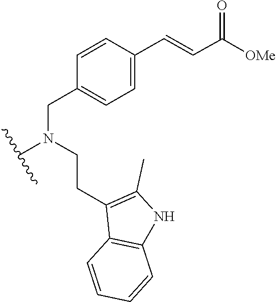 [SMILES: *N(CCc1c(C)[nH]c2ccccc12)Cc1ccc(/C=C/C(=O)OC)cc1]

| Formula | L | n |
|---|---|---|
| No. | | |
| 1103 | 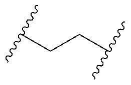 [SMILES: *CC*] | — |
| 1104 | 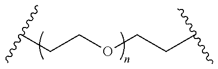 | 1 |
| 1105 | 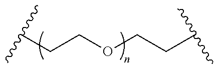 | 2 |
| 1106 | 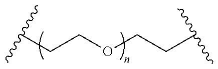 | 3 |
| 1107 | 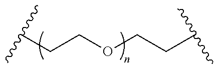 | 4 |
| 1108 | 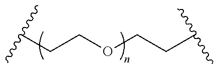 | 5 |

B is

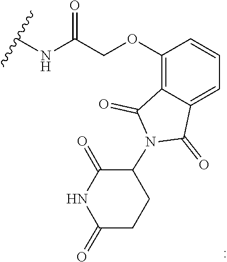 [SMILES: *NC(=O)COc1cccc2c1C(=O)N(C1CCC(=O)NC1=O)C2=O]

| Formula | | |
|---|---|---|
| No. | L | n |
| 1109 | 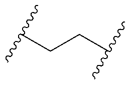 [SMILES: *CC*] | — |
| 1110 | 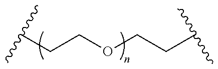 | 1 |
| 1111 | 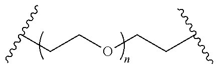 | 2 |
| 1112 | 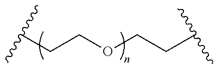 | 3 |
| 1113 | 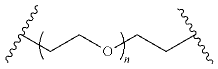 | 4 |
| 1114 | 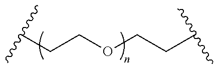 | 5 |

B is

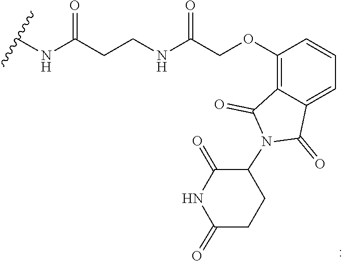 [SMILES: *NC(=O)CCNC(=O)COc1cccc2c1C(=O)N(C1CCC(=O)NC1=O)C2=O]

| Formula | | |
|---|---|---|
| No. | L | n |
| 1115 | 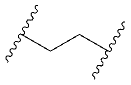 [SMILES: *CC*] | — |
| 1116 | 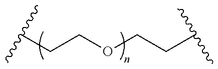 | 1 |
| 1117 | 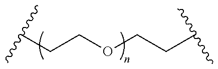 | 2 |
| 1118 | 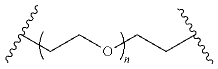 | 3 |
| 1119 | 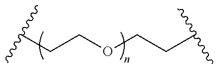 | 4 |
| 1120 | 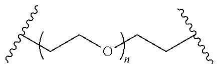 | 5 |

B is

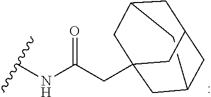 [SMILES: *NC(=O)CC12CC(C)CC(CC(CC)C1)C2]

| Formula | | |
|---|---|---|
| No. | L | n |
| 1121 | 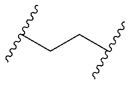 [SMILES: *CC*] | — |
| 1122 | 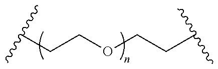 | 1 |
| 1123 | 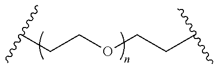 | 2 |
| 1124 | 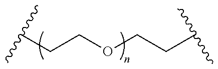 | 3 |
| 1125 | 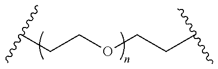 | 4 |
| 1126 | 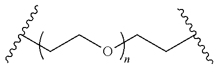 | 5 |

B is

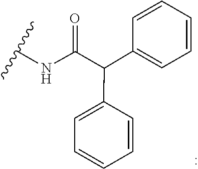 [SMILES: *NC(=O)C(c1ccccc1)c1ccccc1]

| Formula | | |
|---|---|---|
| No. | L | n |
| 1127 | 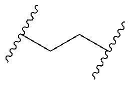 [SMILES: *CC*] | — |
| 1128 | 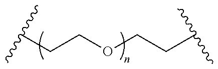 | 1 |
| 1129 | 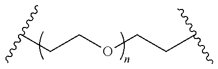 | 2 |
| 1130 | 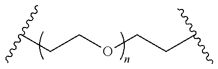 | 3 |
| 1131 | 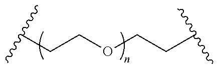 | 4 |
| 1132 | 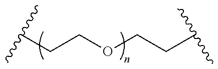 | 5 |

B is

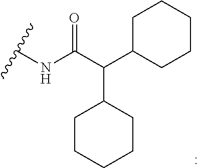 [SMILES: *NC(=O)C(C1CCCCC1)C1CCCCC1]

| Formula | | |
|---|---|---|
| No. | L | n |
| 1133 | 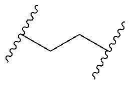 [SMILES: *CC*] | — |
| 1134 | 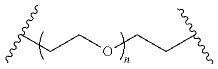 | 1 |
| 1135 | 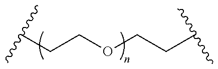 | 2 |
| 1136 | 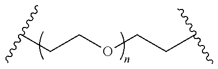 | 3 |
| 1137 | 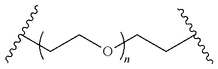 | 4 |
| 1138 | 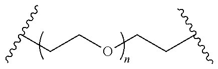 | 5 |

B is

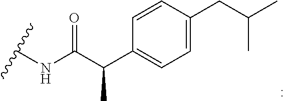 [SMILES: *NC(=O)[C@H](C)c1ccc(CC(C)C)cc1]

| Formula | | |
|---|---|---|
| No. | L | n |
| 1139 | 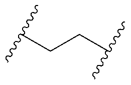 [SMILES: *CC*] | — |
| 1140 | 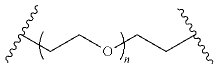 | 1 |
| 1141 | 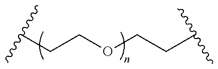 | 2 |
| 1142 | 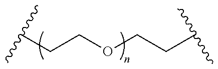 | 3 |
| 1143 | 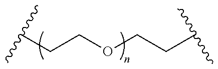 | 4 |
| 1144 | 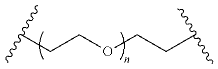 | 5 |

B is

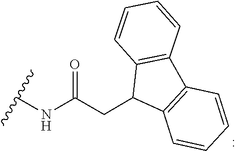 [SMILES: *NC(=O)CC1c2ccccc2-c2ccccc21]

| Formula | | |
|---|---|---|
| No. | L | n |
| 1145 | 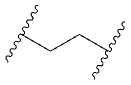 [SMILES: *CC*] | — |
| 1146 | 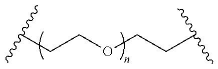 | 1 |
| 1147 | 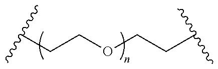 | 2 |
| 1148 | 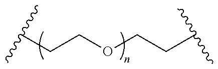 | 3 |
| 1149 | 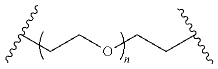 | 4 |
| 1150 | 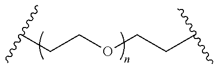 | 5 |

B is

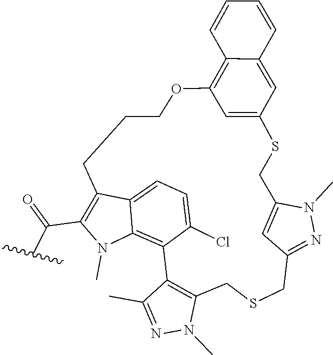 [SMILES: *C(=O)c1c2c3ccc(Cl)c(c3n1C)-c1c(C)nn(C)c1CSCc1cc(n(C)n1)CSc1cc(c3ccccc3c1)OCCC2]

| Formula | | |
|---|---|---|
| No. | L | n |
| 1151 | 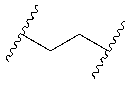 [SMILES: *CC*] | — |
| 1152 | 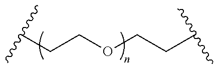 | 1 |
| 1153 | 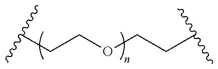 | 2 |
| 1154 | 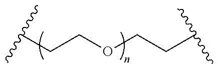 | 3 |
| 1155 | 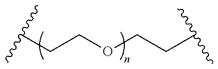 | 4 |
| 1156 | 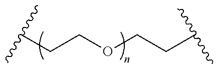 | 5 |

B is

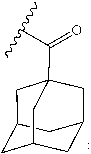 [SMILES: *C(=O)C12CC3CC(CC(C3)C1)C2]

| Formula | | |
|---|---|---|
| No. | L | n |
| 1157 | 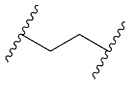 [SMILES: *CC*] | — |
| 1158 | 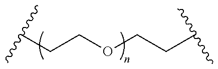 | 1 |
| 1159 | 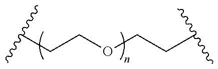 | 2 |
| 1160 | 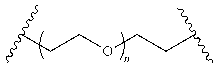 | 3 |
| 1161 | 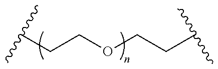 | 4 |
| 1162 | 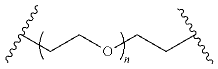 | 5. |

34. The compound of claim 3, wherein the compound of formula (110) is a compound of any one of formula 1163-1170, or a pharmaceutically acceptable salt, solvate, hydrate, cocrystal, or prodrug thereof: 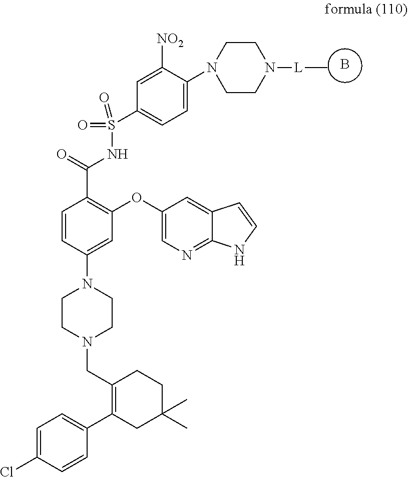
B is

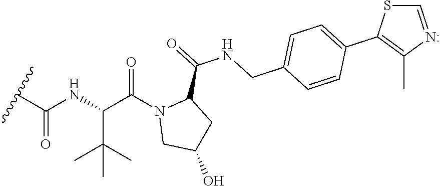

| Formula | | |
|---|---|---|
| No. | L | n |
| 1163 | 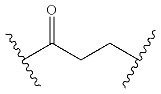 [SMILES: *CCC(*)=O] | — |
| 1164 | 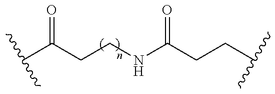 | 0 |
| 1165 | 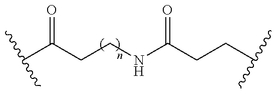 | 1 |
| 1166 | 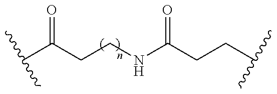 | 2 |
| 1167 | 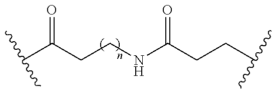 | 3 |
| 1168 | 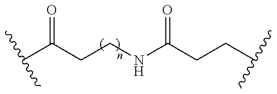 | 4 |
| 1169 | 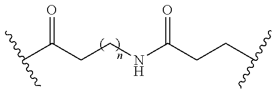 | 5 |
| 1170 | 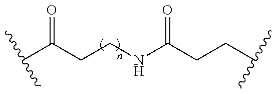 | 6. |

35. A pharmaceutical composition comprising one or more of compounds of any one of claims 1-34 or a pharmaceutically acceptable salt thereof, and a physiologically compatible carrier medium.

36. A pharmaceutical composition for treating or preventing a disease or disorder alleviated by inhibiting Bcl-2 protein activity, the pharmaceutical composition comprising one or more compounds according to any one of claims 1-34, or a pharmaceutically acceptable salt thereof, and a physiologically compatible carrier medium.

37. A pharmaceutical composition for treating or preventing a disease or disorder alleviated by inhibiting and/or downregulating Mcl-1 protein activity, the pharmaceutical composition comprising one or more compounds according to any one of claims 1-34, or a pharmaceutically acceptable salt thereof, and a physiologically compatible carrier medium.

38. A pharmaceutical composition for treating or preventing a disease or disorder alleviated by both inhibiting Bcl-2 protein activity and inhibiting and/or downregulating Mcl-1 protein activity, the pharmaceutical composition comprising one or more compounds according to any one of claims 1-34, or a pharmaceutically acceptable salt thereof, and a physiologically compatible carrier medium.

39. The pharmaceutical composition of any one of claims 36-38, wherein the disease or disorder is cancer.

40. The pharmaceutical composition of claim 39, wherein the cancer is selected from acute myeloid leukemia (AML), chronic lymphocytic leukemia (CLL), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

41. The pharmaceutical composition of claim 39, wherein the cancer is a blood cancer.

42. The pharmaceutical composition of claim 37, wherein the blood cancer is selected from acute myeloid leukemia (AML), chronic lymphocytic leukemia (CLL), chronic myeloid leukemia (CML), acute lymphocytic lymphoma (ALL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

43. The pharmaceutical composition of any one of claims 39-42, wherein the cancer is acute myeloid leukemia (AML).

44. The pharmaceutical composition of any one of claims 39-42, wherein the cancer is chronic lymphocytic leukemia (CLL).

45. A pharmaceutical composition for treating or preventing from acute myeloid leukemia (AML), the pharmaceutical composition comprising one or more compounds according to any one of claims 1-34, or a pharmaceutically acceptable salt thereof, and a physiologically compatible carrier medium.

46. A pharmaceutical composition for treating or preventing from chronic lymphocytic leukemia (CLL), the pharmaceutical composition comprising one or more compounds according to any one of claims 1-34, or a pharmaceutically acceptable salt thereof, and a physiologically compatible carrier medium.

47. A method of treating or preventing a disease or disorder alleviated by inhibiting Bcl-2 protein activity in a patient in need of said treatment or prevention, the method comprising administering a therapeutically effective amount of one or more compounds of any one of claims 1-34, or a pharmaceutically acceptable salt thereof.

48. A method of treating or preventing a disease or disorder alleviated by inhibiting and/or downregulating Mcl-1 protein activity in a patient in need of said treatment or prevention, the method comprising administering a therapeutically effective amount of one or more compounds of any one of claims 1-34, or a pharmaceutically acceptable salt thereof.

49. A method of treating or preventing a disease or disorder alleviated by both inhibiting Bcl-2 protein activity and inhibiting and/or downregulating Mcl-1 protein activity in a patient in need of said treatment or prevention, the method comprising administering a therapeutically effective amount of one or more compounds of any one of claims 1-34, or a pharmaceutically acceptable salt thereof.

50. The method of any one of claims 47-49, wherein the disease or disorder is cancer.

51. The method of claim 50, wherein the cancer is selected from acute myeloid leukemia (AML), chronic lymphocytic leukemia (CLL), chronic myeloid leukemia (CML), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

52. The method of claim 50, wherein the cancer is a blood cancer.

53. The method of claim 52, wherein the blood cancer is selected from acute myeloid leukemia (AML), chronic lymphocytic leukemia (CLL), chronic myeloid leukemia (CML), acute lymphocytic lymphoma (ALL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

54. The method of any one of claims 50-53, wherein the cancer is acute myeloid leukemia (AML).

55. The method of any one of claims 50-53, wherein the cancer is chronic lymphocytic leukemia (CLL).

56. A method of treating or preventing acute myeloid leukemia (AML) in a patient in need of said treatment or prevention, the method comprising administering a therapeutically effective amount of one or more compounds of any one of claims 1-34, or a pharmaceutically acceptable salt thereof.

57. A method of treating or preventing chronic lymphocytic leukemia (CLL) in a patient in need of said treatment or prevention, the method comprising administering a therapeutically effective amount of one or more compounds of any one of claims 1-34, or a pharmaceutically acceptable salt thereof.


SEQUENCE LISTING (1 sequence)

SEQ ID NO: 1 - protein, 16 aa, Artificial Sequence
  Chemically synthesized.
GQVGRQLAIIGDDINR